SEQUENCE LISTING (149 sequences)

SEQ ID NO: 1 - protein, 191 aa, Arabidopsis thaliana
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSIIGGEHRLTNYKSVTTVHRFEKENRIWTVVLESYVVDMPEGNSEDDTRMFADTVVKLNLQKLATVAEAMARNSGDGSGSQVT

SEQ ID NO: 2 - protein, 221 aa, Arabidopsis thaliana
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 3 - protein, 190 aa, Arabidopsis thaliana
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 4 - protein, 209 aa, Arabidopsis thaliana
MNLAPIHDPSSSSTTTTSSSTPYGLTKDEFSTLDSIIRTHHTFPRSPNTCTSLIAHRVDAPAHAIWRFVRDFANPNKYKHFIKSCTIRVNGNGIKEIKVGTIREVSVVSGLPASTSVEILEVLDEEKRILSFRVLGGEHRLNNYRSVTSVNEFVVLEKDKKKRVYSVVLESYIVDIPQGNTEEDTRMFVDTVVKSNLQNLAVISTASPT

SEQ ID NO: 5 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 6 - protein, 203 aa, Arabidopsis thaliana
MRSPVQLQHGSDATNGFHTLQPHDQTDGPIKRVCLTRGMHVPEHVAMHHTHDVGPDQCCSSVVQMIHAPPESVWALVRRFDNPKVYKNFIRQCRIVQGDGLHVGDLREVMVVSGLPAVSSTERLEILDEERHVISFSVVGGDHRLKNYRSVTTLHASDDEGTVVVESYIVDVPPGNTEEETLSFVDTIVRCNLQSLARSTNRQ

SEQ ID NO: 7 - protein, 215 aa, Arabidopsis thaliana
MPTSIQFQRSSTAAEAANATVRNYPHHHQKQVQKVSLTRGMADVPEHVELSHTHVVGPSQCFSVVVQDVEAPVSTVWSILSRFEHPQAYKHFVKSCHVVIGDGREVGSVREVRVVSGLPAAFSLERLEIMDDDRHVISFSVVGGDHRLMNYKSVTTVHESEEDSDGKKRTRVVESYVVDVPAGNDKEETCSFADTIVRCNLQSLAKLAENTSKFS

SEQ ID NO: 8 - protein, 211 aa, Arabidopsis thaliana
MEMIGGDDTDTEMYGALVTAQSLRLRHLHHCRENQCTSVLVKYIQAPVHLVWSLVRRFDQPQKYKPFISRCTVNGDPEIGCLREVNVKSGLPATTSTERLEQLDDEEHILGINIIGGDHRLKNYSSILTVHPEMIDGRSGTMVMESFVVDVPQGNTKDDTCYFVESLIKCNLKSLACVSERLAAQDITNSIATFCNASNGYREKNHTETNL

SEQ ID NO: 9 - protein, 188 aa, Arabidopsis thaliana
MEANGIENLTNPNQEREFIRRHHKHELVDNQCSSTLVKHINAPVHIVWSLVRRFDQPQKYKPFISRCVVKGNMEIGTVREVDVKSGLPATRSTERLELLDDNEHILSIRIVGGDHRLKNYSSIISLHPETIEGRIGTLVIESFVVDVPEGNTKDETCYFVEALIKCNLKSLADISERLAVQDTTESRV

SEQ ID NO: 10 - protein, 187 aa, Arabidopsis thaliana
MMDGVEGGTAMYGGLETVQYVRTHHQHLCRENQCTSALVKHIKAPLHLVWSLVRRFDQPQKYKPFVSRCTVIGDPEIGSLREVNVKSGLPATTSTERLELLDDEEHILGIKIIGGDHRLKNYSSILTVHPEIIEGRAGTMVIESFVVDVPQGNTKDETCYFVEALIRCNLKSLADVSERLASQDITQ

SEQ ID NO: 11 - protein, 183 aa, Arabidopsis thaliana
MNGDETKKVESEYIKKHHRHELVESQCSSTLVKHIKAPLHLVWSIVRRFDEPQKYKPFISRCVVQGKKLEVGSVREVDLKSGLPATKSTEVLEILDDNEHILGIRIVGGDHRLKNYSSTISLHSETIDGKTGTLAIESFVVDVPEGNTKEETCFFVEALIQCNLNSLADVTERLQAESMEKKI

SEQ ID NO: 12 - protein, 160 aa, Arabidopsis thaliana
METSQKYHTCGSTLVQTIDAPLSLVWSILRRFDNPQAYKQFVKTCNLSSGDGGEGSVREVTVVSGLPAEFSRERLDELDDESHVMMISIIGGDHRLVNYRSKTMAFVAADTEEKTVVVESYVVDVPEGNSEEETTSFADTIVGFNLKSLAKLSERVAHLK

SEQ ID NO: 13 - protein, 159 aa, Arabidopsis thaliana
MKTSQEQHVCGSTVVQTINAPLPLVWSILRRFDNPKTFKHFVKTCKLRSGDGGEGSVREVTVVSDLPASFSLERLDELDDESHVMVISIIGGDHRLVNYQSKTTVFVAAEEEKTVVVESYVVDVPEGNTEEETTLFADTIVGCNLRSLAKLSEKMMELT

SEQ ID NO: 14 - protein, 164 aa, Arabidopsis thaliana
MESSKQKRCRSSVVETIEAPLPLVWSILRSFDKPQAYQRFVKSCTMRSGGGGGKGGEGKGSVRDVTLVSGFPADFSTERLEELDDESHVMVVSIIGGNHRLVNYKSKTKVVASPEDMAKKTVVVESYVVDVPEGTSEEDTIFFVDNIIRYNLTSLAKLTKKMMK

SEQ ID NO: 15 - protein, 191 aa, Brassica oleracea
MPSQLTPEERSELAQSIAEFHTYHLGPGSCSSLHAQRIHAPPEIVWSVVRRFDKPQTYKHFIKSCSVEDGFEMRVGCTRAVNVISGLPANTSTERLDILDDERRVTGFSIIGGEHRLTNYKSVTTVHRFEKERRIWTVVLESYVVDMPEGNSEDDTRMFADTVVKLNLQKLATVTEAMARNAGDGSGAQVT

SEQ ID NO: 16 - protein, 281 aa, Brassica oleracea
MPSELTQEERSKLTQSISEFHTYHLGPGSCSSLHAQRIHAPPEIVWSVVRQFDKPQTYKHFIKSCSVEEGFEMRVGCTRDVIVISGLPANTSTERLDMLDDERRVTGFSIIGGEHRLKNYKSVTTVHRFERERRIWTVVLESYVVDMPEGNSEDDTRMFADTVVKLNLQKLATVTEAMARNAGDGRGSRETTCRESFHLITAFEKQRQITEPTVYQNPPYHTGMTPEPRTSTVFIELEDHRTLPGNLTPTTEEHLQRMYQRFWGIRQLQRPRQSFGERQSI

SEQ ID NO: 17 - protein, 453 aa, Vitis vinifera
MQMKYLEGKQNLMEEKGEKQCIPMDLAVREAQFKGSLLDRITWLEQRLHKLSLQLETRSKQQPHPSRMQTAGETSSRHGPKKELSCSFPVFSTRNHNHGHKQTSQFHVPRFEYQEGGRENPAVVITKLTPFHHPKIITILFPISNYFIIFFFLTFDTKKQYPLLFPILPSRFLPISHLITQEIEKYKTSSHFSSPASLFAAMNKAETSSMAEAESEDSETTTPTTHHLTIPPGLTQPEFQELAHSISEFHTYQVGPGQCSSLLAQRVHAPLPTVWSVVRRFDKPQTYKHFIKSCHVEDGFEMRVGCLRDVNVISGLPAETSTERLDILDDERHVTGFSIIGGEHRLRNYRSVTTNHGGEIWTVVLESYVVDMPEGNTEEDTRLFADTVVKLNLQKLASVTEVSQSCNYPCQFHIIENEDIQPEEMNLGVLTTSIEEQRKKKRVVAMKDGSTSS

SEQ ID NO: 18 - protein, 195 aa, Vitis vinifera
  misc_feature (193)..(193): Xaa can be any naturally occurring amino acid
MAEAESEDSETTTPTTHHLTIPPGLTQPEFQELAHSISEFHTYQVGPGQCSSLLAQRVHAPLPTVWSVVRRFDKPQTYKHFIKSCHVEDGFEMRVGCLRDVNVISGLPAETSTERLDILDDERHVTGFSIIGGEHRLRNYRSVTTVHEYQNHGGEIWTVVLESYVVDMPEGNTEEDTRLFADTVVKLNLSEAXRR

SEQ ID NO: 19 - protein, 217 aa, Medicago truncatula
MEKAESSTASTSDQDSDENHRTQHHLTLPSGLRQHEFDSLIPFINSHHTYLIGPNQCSTLLAQRIHAPPQTVWSVVRSFDKPQIYKHIIKSCSLKEGFQMKVGCTRDVNVISGLPAATSTERLDVLDDERRVTGFSIIGGEHRLKNYRSVTSVHGFGDGDNGGEIWTVVLESYVVDVPEGNTEEDTRLFADTVVKLNLQKLASVTEGKNRDGDGKSH

SEQ ID NO: 20 - protein, 212 aa, Oryza sativa
MEQQEEVPPPPAGLGLTAEEYAQVRATVEAHHRYAVGPGQCSSLLAQRIHAPPAAVWAVVRRFDCPQVYKHFIRSCVLRPDPHHDDNGNDLRPGRLREVSVISGLPASTSTERLDLLDDAHRVFGFTITGGEHRLRNYRSVTTVSQLDEICTLVLESYIVDVPDGNTEDDTRLFADTVIRLNLQKLKSVSEANANAAAAAAAPPPPPPAAAE

SEQ ID NO: 21 - protein, 212 aa, Zea mays
MDQQGAGGDAEVPAGLGLTAAEYEQLRSTVDAHHRYAVGEGQCSSLLAQRIHAPPEAVWAVVRRFDCPQVYKHFIRSCALRPDPEAGDALCPGRLREVSVISGLPASTSTERLDLLDDAARVFGFSITGGEHRLRNYRSVTTVSELAVPAICTVVLESYVVDVPDGNTEDDTRLFADTVIRLNLQKLKSVAEANAAEAAATTNSVLLPRPAE

SEQ ID NO: 22 - protein, 212 aa, Zea mays
  misc_feature (11)..(11): Xaa can be any naturally occurring amino acid
MDQQGAGGDAXVPAGLGLTAAEYEQLRSTVDAHHRYAVGEGQCSSLLAQRIHAPPEAVWAVVRRFDCPQVYKHFIRSCALRPDPEAGDALCPGRLREVSVISGLPASTSTERLDLLDDAARVFGFSITGGEHRLRNYRSVTTVSELADPAICTVVLESYVVDVPDGNTEDDTRLFADTVIRLNLQKLKSVTEANAAEAAATTNSVLLPRPAE

SEQ ID NO: 23 - protein, 233 aa, Vitis vinifera
MDPHHHHGLTEEEFRALEPIIQNYHTFEPSPNTCTSLITQKIDAPAQVVWPFVRSFENPQKYKHFIKDCTMRGDGGVGSIREVTVVSGLPASTSTERLEILDDEKHILSFRVVGGEHRLNNYRSVTSVNDFSKEGKDYTIVLESYIVDIPEGNTGEDTKMFVDTVVKLNLQKLAVVAITSLHENEEIADNEGPSREISLQSETESAERGDERRDGDGPSKACNRNEWHCTTKE

SEQ ID NO: 24 - protein, 207 aa, Oryza sativa
MEPHMERALREAVASEAERRELEGVVRAHHTFPAAERAAGPGRRPTCTSLVAQRVDAPLAAVWPIVRGFANPQRYKHFIKSCELAAGDGATVGSVREVAVVSGLPASTSTERLEILDDDRHVLSFRVVGGDHRLRNYRSVTSVTEFSSPSSPPRPYCVVVESYVVDVPEGNTEEDTRMFTDTVVKLNLQKLAAVATSSSPPAAGNHH

SEQ ID NO: 25 - protein, 210 aa, Oryza sativa
MEPHMERALREAVASEAERRELEGVVRAHHTFPAAERAAGPGRRPTCTSLVAQRVDAPLAAVWPIVRGFANPQRYKHFIKSCELAAGDGATVGSVREVAVVSGLPASTSTERLEILDDDRHVLSFRVVGGDHRLRNYRSVTSVTEFSSPSSPPSPPRPYCVVVESYVVDVPEGNTEEDTRMFTDTVVKLNLQKLAAVATSSSPPAAGNHH

SEQ ID NO: 26 - protein, 200 aa, Zea mays
MPYTAPRPSPQQHSRVLSGGGAKAASHGASCAAVPAEVARHHEHAARAGQCCSAVVQAIAAPVGAVWSVVRRFDRPQAYKHFIRSCRLVGGGDVAVGSVREVRVVSGLPATSSRERLEILDDERRVLSFRVVGGEHRLANYRSVTTVHEAGAGAGTGTVVVESYVVDVPHGNTADETRVFVDTIVRCNLQSLARTAERLA

SEQ ID NO: 27 - protein, 215 aa, Vitis vinifera
MPSNPPKSSLVVHRINSPNSITTATTASAAANNHNTSTMPPHKQVPDAVSRHHTHVVGPNQCCSAVVQQIAAPVSTVWSVVRRFDNPQAYKHFVKSCHVVVGDGDVGTLREVHVISGLPAANSTERLEILDDERHVLSFSVIGGDHRLSNYRSVTTLHPSPSSTGTVVLESYVVDIPPGNTKEDTCVFVDTIVRCNLQSLAQIAENAAGCKRSSS

SEQ ID NO: 28 - protein, 213 aa, Nicotiana tabacum
MPPSSPDSSVLLQRISSNTTPDFACKQSQQLQRRTMPIPCTTQVPDSVVRFHTHPVGPNQCCSAVIQRISAPVSTVWSVVRRFDNPQAYKHFVKSCHVIVGDGDVGTLREVRVISGLPAASSTERLEILDDERHVISFSVVGGDHRLANYRSVTTLHPEPSGDGTTIVVESYVVDVPPGNTRDETCVFVDTIVKCNLTSLSQIAVNVNRRKDS

SEQ ID NO: 29 - protein, 208 aa, Oryza sativa
MPYAAVRPSPPPQLSRPIGSGAGGGKACPAVPCEVARYHEHAVGAGQCCSTVVQAIAAPADAVWSVVRRFDRPQAYKKFIKSCRLVDGDGGEVGSVREVRVVSGLPATSSRERLEVLDDDRRVLSFRIVGGEHRLANYRSVTTVHEAAAPAMAVVVESYVVDVPPGNTWEETRVFVDTIVRCNLQSLARTVERLAPEAPRANGSIDHA

SEQ ID NO: 30 - protein, 208 aa, Oryza sativa
MPYAAVRPSPPPQLSRPIGSGAGGGKACPAVPCEVARYHEHAVGAGQCFSTVVQAIAAPADAVWSVVRRFDRPQAYKKFIKSCRLVDGDGGEVGSVREVRVVSGLPATSSRERLEVLDDDRRVLSFRIVGGEHRLANYRSVTTVHEAAAPAMAVVVESYVVDVPPGNTWEETRVFVDTIVRCNLQSLARTVERLAPEAPRANGSIDHA

SEQ ID NO: 31 - protein, 213 aa, Picea sitchensis
MDIIAGFDQLSFRLSGASKQITKTGAVQYLKGEEGYGEWLKEVMGRYHYHSHDGARECRCSSVVVQQVEAPVSVVWSLVRRFDQPQVYKHFVSNCFMRGDLKVGCLREVRVVSGLPAATSTERLDILDEERHILSFSIVGGDHRLNNYRSITTLHETLINGKPGTIVIESYVLDVPHGNTKEETCLFVDTIVKCNLQSLAHVSNHLNSTHRCL

SEQ ID NO: 32 - protein, 207 aa, Oryza sativa
MEAHVERALREGLTEEERAALEPAVMAHHTFPPSTTTATTAAATCTSLVTQRVAAPVRAVWPIVRSFGNPQRYKHFVRTCALAAGDGASVGSVREVTVVSGLPASTSTERLEMLDDDRHIISFRVVGGQHRLRNYRSVTSVTEFQPPAAGPGPAPPYCVVVESYVVDVPDGNTAEDTRMFTDTVVKLNLQMLAAVAEDSSSASRRRD

SEQ ID NO: 33 - protein, 216 aa, Oryza sativa
MPYTAPRPSPPQHSRIGGCGGGGVLKAAGAAGHAASCVAVPAEVARHHEHAAGVGQCCSAVVQAIAAPVDAVWSVVRRFDRPQAYKHFIRSCRLLDGDGDGGAVAVGSVREVRVVSGLPATSSRERLEILDDERRVLSFRVVGGEHRLSNYRSVTTVHETAAGAAAAVVVESYVVDVPHGNTADETRMFVDTIVRCNLQSLARTAEQLALAAPRAA

SEQ ID NO: 34 - protein, 212 aa, Vitis vinifera
MPSSLQLHRINNIDPTTVAVAATAAVNCHKQSRTPLRCATPVPDAVASYHAHAVGPHQCCSMVVQTTAAALPTVWSVVRRFDNPQAYKHFLKSCHVIFGDGDIGTLREVHVVSGLPAESSTERLEILDDERHVLSFSVVGGDHRLCNYRSVTTLHPSPTGTGTVVVESYVVDIPPGNTKEDTCVFVDTIVKCNLQSLAQMSEKLTNNNRNSS

SEQ ID NO: 35 - protein, 218 aa, Zea mays
MPCLQASSPGSMPYQHHGRGVGCAAEAGAAVGASAGTGTRCGAHDGEVPAEAARHHEHAAPGPGRCCSAVVQRVAAPAEAVWSVVRRFDQPQAYKRFVRSCALLAGDGGVGTLREVRVVSGLPAASSRERLEVLDDESHVLSFRVVGGEHRLQNYLSVTTVHPSPAAPDAATVVVESYVVDVPPGNTPEDTRVFVDTIVKCNLQSLATTAEKLALAAV

SEQ ID NO: 36 - protein, 179 aa, Physcomitrella patens
MQTKGRQADFQTLLEGQQDLICRFHRHELQPHQCGSILLQLIKAPVETVWSVARSFDKPQVYKRFIQTCEIIEGDGGVGSIREVRLVSSIPATSSIERLEILDDEEHIISFRVLGGGHRLQNYWSVTSLHSHEIDGQMGTLVLESYVVDIPEGNTREETHMFVDTVVRCNLKALAQVSE

SEQ ID NO: 37 - protein, 229 aa, Oryza sativa
MPCIPASSPGIPHQHQHQHHRALAGVGMAVGCAAEAAVAAAGVAGTRCGAHDGEVPMEVARHHEHAEPGSGRCCSAVVQHVAAPAPAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 38 - protein, 229 aa, Oryza sativa
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 39 - protein, 205 aa, Medicago truncatula
MPSPVQFQRFDSNTAITNGVNCPKQIQACRYALSSLKPTVSVPETVVDHHMHVVGQNQCYSVVIQTINASVSTVWSVVRRFDYPQGYKHFVKSCNVVASGDGIRVGALREVRLVSGLPAVSSTERLDILDEERHVISFSVVGGVHRCRNYRSVTTLHGDGNGGTVVIESYVVDVPQGNTKEETCSFADTIVRCNLQSLVQIAEKL

SEQ ID NO: 40 - protein, 212 aa, Zea mays
MPFAASRTSQQQHSRVATNGRAVAVCAGHAGVPDEVARHHEHAVAAGQCCAAMVQSIAAPVDAVWSLVRRFDQPQRYKRFIRSCHLVDGDGAEVGSVRELLLVSGLPAESSRERLEIRDDERRVISFRVLGGDHRLANYRSVTTVHEAAPSQDGRPLTMVVESYVVDVPPGNTVEETRIFVDTIVRCNLQSLEGTVIRQLEIAAMPHDDNQN

SEQ ID NO: 41 - protein, 233 aa, Zea mays
MRERNSSIDQEHQRGSSSRSTMPFAASRTSQQQHSRVATNGRAVAVCAGHAGVPDEVARHHEHAVAAGQCCAAMVQSIAAPVDAVWSLVRRFDQPQRYKRFIRSCHLVDGDGAEVGSVRELLLVSGLPAESSRERLEIRDDERRVISFRVLGGDHRLANYRSVTTVHEAAPSQDGRPLTMVVESYVVDVPPGNTVEETRIFVDTIVRCNLQSLEGTVIRQLEIAAMPHDDNQN

SEQ ID NO: 42 - protein, 194 aa, Physcomitrella patens
MMQEKQGRPDFQFLLEGQQDLICRFHKHELLPHQCGSILLQQIKAPVQTVWLIVRRFDEPQVYKRFIQRCDIVEGDGVVGSIREVQLVSSIPATSSIERLEILDDEEHIISFRVLGGGHRLQNYWSVTSLHRHEIQGQMGTLVLESYVVDIPDGNTREETHTFVDTVVRCNLKALAQVSEQKHLLNSNEKPAAP

SEQ ID NO: 43 - protein, 191 aa, Vitis vinifera
MKVYSPSQILAERGPRAQAMGNLYHTHHLLPNQCSSLVVQTTDAPLPQVWSMVRRFDRPQSYKRFVRGCTLRRGKGGVGSVREVNIVSGLPAEISLERLDKLDDDLHVMRFTVIGGDHRLANYHSTLTLHEDEEDGVRKTVVMESYVVDVPGGNSAGETCYFANTIIGFNLKALAAVTETMALKANIPSGF

SEQ ID NO: 44 - protein, 217 aa, Physcomitrella patens
MQQVKGRQDFQRLLEAQQDLICRYHTHELKAHQCGSILLQQIKVPLPIVWAIVRSFDKPQVYKRFIQTCKITEGDGGVGSIREVHLVSSVPATCSIERLEILDDEKHIISFRVLGGGHRLQNYSSVSSLHELEVEGHPCTLVLESYMVDIPDGNTREETHMFVDTVVRCNLKSLAQISEQQYNKDCLQQKQHDQQQMYQQRHPPLPPIPITDKNMER

SEQ ID NO: 45 - protein, 195 aa, Physcomitrella patens
MRFDIGHNDVRGFFTCEEEHAYALHSQTVELNQCGSILMQQIHAPIEVVWSIVRSFGSPQIYKKFIQACILTVGDGGVGSIREVFLVSGVPATSSIERLEILDDEKHVFSFRVLKGGHRLQNYRSVTTLHEQEVNGRQTTTVLESYVVDVPDGNTREETHMFADTVVMCNLKSLAQVAEWRAMQGITQQLSTSSL

SEQ ID NO: 46 - protein, 172 aa, Vitis vinifera
MGNLYHTHHLLPNQCSSLVVQTTDAPLPQVWSMVRRFDRPQSYKRFVRGCTLRRGKGGVGSVREVNIVSGLPAEISLERLDKLDDDLHVMRFTVIGGDHRLANYHSTLTLHEDEEDGVRKTVVMESYVVDVPGGNSAGETCYFANTIIGFNLKALAAVTETMALKANIPSGF

SEQ ID NO: 47 - protein, 196 aa, Picea sitchensis
MEDLSSWREGRAMWLGNPPSESELVCRHHRHELQGNQCSSFLVKHIRAPVHLVWSIVRTFDQPQKYKPFVHSCSVRGGITVGSIRNVNVKSGLPATASEERLEILDDNEHVFSIKILGGDHRLQNYSSIITVHPEIIDGRPGTLVIESYVVDVPEGNTREETRFFVEALVKCNLKSLADVSERLASQHHTELLERT

SEQ ID NO: 48 - protein, 185 aa, Solanum tuberosum
MNANGFCGVEKEYIRKHHLHEPKENQCSSFLVKHIRAPVHLVWSLVRRFDQPQKYKPFISRCIVQGDLEIGSLREVDVKSGLPATTSTERLELLDDEEHILSVRIVGGDHRLRNYSSVISVHPEVIDGRPGTVVLESFVVDVPEGNTKDETCYFVEALINCNLKSLADISERVAVQDRTEPIDQV

SEQ ID NO: 49 - protein, 190 aa, Medicago truncatula
MNNGCEQQQYSVIETQYIRRHHKHDLRDNQCSSALVKHIKAPVHLVWSLVRRFDQPQKYKPFISRCIMQGDLSIGSVREVNVKSGLPATTSTERLEQLDDEEHILGIRIVGGDHRLRNYSSIITVHPGVIDGRPGTMVIESFVVDVPEGNTKDETCYFVEALIRYNLSSLADVSERMAVQGRTDPININP

SEQ ID NO: 50 - protein, 185 aa, Vitis vinifera
MSGYGCIKMEDEYIRRHHRHEIRDNQCSSSLVKHIKAPVHLVWSLVRSFDQPQKYKPFVSRCIVQGDLEIGSVREVNVKSGLPATTSTERLELLDDEEHIFGMRIVGGDHRLKNYSSIVTVHPEIIDGRPGTLVIESFVVDVPDGNTKDETCYFVEALIKCNLKSLADVSERLAIQDRTEPIDRM

SEQ ID NO: 51 - protein, 185 aa, Vitis vinifera
MNGNGLSSMESEYIRRHHRHEPAENQCSSALVKHIKAPVPLVWSLVRRFDQPQKYKPFISRCVVQGNLEIGSLREVDVKSGLPATTSTERLELLDDDEHILSMRIIGGDHRLRNYSSIISLHPEIIDGRPGTMVIESYVVDVPEGNTKDETCYFVEALIKCNLKSLADVSERLAVQDRTEPIDRM

SEQ ID NO: 52 - protein, 208 aa, Oryza sativa
MEAHVERALREGLTEEERAALEPAVMAHHTFPPSTTTATTAAATCTSLVTQRVAAPVRAVWPIVRSFGNPQRYKHFVRTCALAAGNGPSFGSVREVTVVSGPSRLPPGTERLEMLDDDRHIISFRVVGGQHRLRNYRSVTSVTEFQPPAAGPGPAPPYCVVVESYVVDVPDGNTAEDTRMFTDTVVKLNLQMLAAVAEDSSSASRRRD

SEQ ID NO: 53 - protein, 186 aa, Capsicum annuum
MMNANGFSGVEKEYIRKHHLHQPKENQCSSFLVKHIRAPVHLVWSLVRRFDQPQKYKPFVSRCIAQGDLEIGSLREVDVKSGLPATTSTERLELLDDEEHILSFRIIGGDHRLRNYSSIISLHPEVIDGRPGTLVIESFVVDVPQGNTKDETCYFVEALINCNLKSLADVSERLAVQDRTEPIDQV

SEQ ID NO: 54 - protein, 186 aa, Populus trichocarpa
MNGSDAYSATEAQYVRRHHKHEPRENQCTSALVKHIKAPAHLVWSLVRRFDQPQRYKPFVSRCVMNGELGIGSVREVNVKSGLPATTSTERLELLDDEEHILGVQIVGGDHRLKNYSSIMTVHPEFIDGRPGTLVIESFIVDVPDGNTKDETCYFVEALIRCNLKSLADVSERMAVQDRVEPVNQF

SEQ ID NO: 55 - protein, 185 aa, Capsicum annuum
MNANGFSGVEKEYIRKHHLHQPKENQCSSFLVKHIRAPVHLVWSLVRRFDQPQKYKPFVSRCIAQGDLEIGSLREVDVKSGLPATTSTERLELLDDEEHILSFRIIGGDHRLRNYSSIISLHPEVIDGRPGTLVIESFVVDVPQGNTKDETCYFVEALINCNLKSLADVSERLAVQDRTEPIDQV

SEQ ID NO: 56 - protein, 186 aa, Populus deltoides
MNGSDAYSATEAQYVRRHHKHEPRENQCTSALVKHIKAPAHLVWSLVRRFDQPQRYKPFVSRCVMNGELGIGSVREVNVKSGLPATTSTERLELLDDEEHILGVQIVGGDHRLKNYSSIMTVHPEFIDGRPGTLVIESFIVDVPDGNTKDETCYFVKALIRCNLKSLADVSERMAVQDRVEPVNQF

SEQ ID NO: 57 - protein, 188 aa, Pisum sativum
MNNGGEQYSAIETQYIRRRHKHDLRDNQCSSALVKHIKAPVHLVWSLVRRFDQPQKYKPFVSRCIMQGDLGIGSVREVNVKSGLPATTSTERLEQLDDEEHILGIRIVGGDHRLRNYSSVITVHPEVIDGRPGTMVIESFVVDVPEGNTRDETCYFVEALIRGNLSSLADVSERMAVQGRTDPINVNP

SEQ ID NO: 58 - protein, 176 aa, Vitis vinifera
MEAQVICRHHAHEPRENQCSSVLVRHVKAPANLVWSLVRRFDQPQKYKPFVSRCVVQGDLRIGSVREVNVKTGLPATTSTERLELFDDDEHVLGIKILDGDHRLRNYSSVITVHPEIIDGRPGTLVIESFVVDVPEGNTKDDTCYFVRALINCNLKCLAEVSERMAMLGRVEPANA

SEQ ID NO: 59 - protein, 176 aa, Vitis vinifera
MMEAQVICRHHAHEPRENQCSSVLVRHVKAPANLVWSLVRRFDQPQKYKPFVSRCVVQGDLRIGSVREVNVKTGLPATTSTERLELFDDDEHVLGIKILDGDHRLRNYSSVITVHPEIIDGRPGTLVIESFVVDVPEGNTKDDTCYFVRALINCNLKCLAEVSERMAMLGRVEPAN

SEQ ID NO: 60 - protein, 193 aa, Arachis hypogaea
  misc_feature (162)..(162): Xaa can be any naturally occurring amino acid
MMNGSCGGGGGGEAYGAIEAQYIRRHHRHEPRDNQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIEGRPGTMVIESFVVDVPDGNTKDETCXFVEALIRCNLSSLADVSERMAVQGRTDPINQ

SEQ ID NO: 61 - protein, 217 aa, Zea mays
MVVEMDGGVGVAAGGGGGAQTPAPAPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQNYSSILTVHPEVIDGRPGTLVIESFVVDVPDGNTKDETCYFVEALLKCNLRSLAEVSEGQVIMDQTEPLDR

SEQ ID NO: 62 - protein, 217 aa, Zea mays
MVVEMDGGVGVAAAGGGGAQTPAPPPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQNYSSILTVHPEVIDGRPGTLVIESFVVDVPDGNTKDETCYFVEALLKCNLRSLAEVSEGQVIMDQTEPLDR

SEQ ID NO: 63 - protein, 206 aa, Oryza sativa
MNGVGGAGGAAAGKLPMVSHRRVQWRLADERCELREEEMEYIRRFHRHEPSSNQCTSFAAKHIKAPLHTVWSLVRRFDQPQLFKPFVRNCVMRENIIATGCIREVNVQSGLPATRSTERLELLDDNEHILKVNFIGGDHMLKNYSSILTVHSEVIDGQLGTLVVESFIVDVPEGNTKDDISYFIENVLRCNLRTLADVSEERLANP

SEQ ID NO: 64 - protein, 206 aa, Artificial Sequence
  synthetic rice Indica Group, cultivar 93-11 protein
MNGAGGAGGAAAGKLPMVSHRQVQWRLADERCELREEEMEYIRQFHRHEPSSNQCTSFVAKHIKAPLQTVWSLVRRFDQPQLFKPFVRKCVMRENIIATGCVREVNVQSGLPATRSTERLELLDDNEHILKVKFIGGDHMLKNYSSILTIHSEVIDGQLGTLVVESFVVDIPEGNTKDDICYFIENILRCNLMTLADVSEERLANP

SEQ ID NO: 65 - protein, 205 aa, Oryza sativa
MVEVGGGAAEAAAGRRWRLADERCDLRAAETEYVRRFHRHEPRDHQCSSAVAKHIKAPVHLVWSLVRRFDQPQLFKPFVSRCEMKGNIEIGSVREVNVKSGLPATRSTERLELLDDNEHILSVRFVGGDHRLKNYSSILTVHPEVIDGRPGTLVIESFVVDVPEGNTKDETCYFVEALLKCNLKSLAEVSERLVCQGPNRAPSTR

SEQ ID NO: 66 - protein, 204 aa, Oryza sativa
MVEVGGGAAEAAAGRRWRLADERCDLRAAETEYVRRFHRHEPRDHQCSSAVAKHIKAPVHLVWSLVRRFDQPQLFKPFVSRCEMKGNIEIGSVREVNVKSGLPATRSTERLELLDDNEHILSVRFVGGDHRLKNYSSILTVHPEVIDGRPGTLVIESFVVDVPEGNTKDETCYFVEALLKCNLKSLAEVSERLVVKDQTEPLDR

SEQ ID NO: 67 - protein, 199 aa, Medicago truncatula
MEKMNGTENNGVFNSTEMEYIRRHHNQQPGENQCSSALVKHIRAPVPLVWSLVRRFDQPQKYKPFVSRCVVRGNLEIGSLREVDVKSGLPATTSTERLEVLDDNEHILSIRIIGGDHRLRNYSSIMSLHPEIIDGRPGTLVIESFVVDVPEGNTKDETCYFVEALIKCNLKSLSDVSEGHAVQDLTEPLDRVHELLISG

SEQ ID NO: 68 - protein, 199 aa, Medicago truncatula
MEKMNGTENNGVFNSTEMEYIRRHHNQQPGENQCSSALVKHIRAPVPLVWSLVRRFDQPQKYKPFVSRCVVRGNLEIGSLREVDVKSGLPATTSTERLEVLDDNEHILSIRIIGGDHRLRNYSSIMSLHPEIIDGRPGTLVIESFVVDVPEGNTKDETCYFVEALIKCNLKSLSDVSEGHAAQDLTEPLDRMHELLISG

SEQ ID NO: 69 - protein, 197 aa, Zea mays
MVGLVGGSTARAEHVVANAGGEAEYVRRMHRHAPTEHQCTSTLVKHIKAPVHLVWQLVRRFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLNSLAEVSEQLAVESPTSLIDQ

SEQ ID NO: 70 - protein, 197 aa, Zea mays
MVGLVGGSTARAEHVVANAGGEAEYVRRMHRHAPTEHQCTSTLVKHIKAPVHLVWELVRRFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLNSLAEVSEQLAVESPTSLIDQ

SEQ ID NO: 71 - protein, 212 aa, Zea mays
MVMVEMDGGVGGGGGGGQTPAPRRWRLADERCDLRAMETDYVRRFHRHEPREHQCSSAVAKHIKAPVHLVWSLVRRFDQPQLFKPFVSRCEMKGNIEIGSVREVNVKSGLPATRSTERLELLDDNEHILSVRFVGGDHRLQNYSSILTVHPEVIDGRPGTLVIESFVVDVPDGNTKDETCYFVEALLKCNLKSLAEVSERQVVKDQTEPLDR

SEQ ID NO: 72 - protein, 205 aa, Oryza sativa
MNGAGGAGGAAAGKLPMVSHRRVQCRLADKRCELREEEMEYIRQFHRHEPSSNQCTSFVAKHIKAPLQTVWSLVRRFDQPQLFKPFVRKCVMRENIIVTGCVREVNVQSGLPATRSTERLELLDDNEHILKVKFIGGDHMLKNYSSILTIHSEVIDGQLGTLVVESFVVDIPDGNTKDDICYFIENVLRCNLMTLADVSEERLAN

SEQ ID NO: 73 - protein, 197 aa, Zea mays
MVGLVGGSTARAEHVVANAGGETEYVRRLHRHAPAEHQCTSTLVKHIKAPVHLVWELVRSFDQPQRYKPFVRNCVVRGDQLEVGSLRDVNVKTGLPATTSTERLEQLDDDLHILGVKFVGGDHRLQNYSSIITVHPESIDGRPGTLVIESFVVDVPDGNTKDETCYFVEAVIKCNLKSLAEVSEQLAVESPTSPIDQ

SEQ ID NO: 74 - protein, 206 aa, Oryza sativa
MNGVGGAGGAAAGKLPMVSHRRVQWRLADERCELREEEMEYIRRFHRHEPSSNQCTSFAAKHIKAPLHTVWSLVRRFDQPQLFKPFVRNCVMRENIIATGCIREVNVQSGLPATRSTERLELLDDNEHILKVKFIGGDHMLKNYSSILTVHSEVIDGQLGTLVVESFIVDVLEGNTKDDISYFIENVLRCNLRTLADVSEERLANP

SEQ ID NO: 75 - protein, 209 aa, Oryza sativa
MVGLVGGGGWRVGDDAAGGGGGGAVAAGAAAAAEAEHMRRLHSHAPGEHQCSSALVKHIKAPVHLVWSLVRSFDQPQRYKPFVSRCVVRGGDLEIGSVREVNVKTGLPATTSTERLELLDDDEHILSVKFVGGDHRLRNYSSIVTVHPESIDGRPGTLVIESFVVDVPDGNTKDETCYFVEAVIKCNLTSLAEVSERLAVQSPTSPLEQ

SEQ ID NO: 76 - protein, 180 aa, Oryza sativa
MVEMDAGGRPEPSPPSGQCSSAVTMRINAPVHLVWSIVRRFEEPHIFQPFVRGCTMRGSTSLAVGCVREVDFKSGFPAKSSVERLEILDDKEHVFGVRIIGGDHRLKNYSSVLTAKPEVIDGEPATLVSESFVVDVPEGNTADETRHFVEFLIRCNLRSLAMVSQRLLLAQGDLAEPPAQ

SEQ ID NO: 77 - protein, 176 aa, Vitis vinifera
MNGNGLSSMESEYIRRHHRHEPAENQCSSALVKHIKAPVPLVWSLVRRFDQPQKYKPFISRCVVQGNLEIGSLREVDVKSGLPATTSTERLELLDDDEHILSMRIIGGDHRLRNYSSIISLHPEIIDGRPGTMVIESYVVDVPEGNTKDETCYFSLADVSERLAVAGTVTEPIDRM

SEQ ID NO: 78 - protein, 180 aa, Oryza sativa
MVEMDAGGRPEPSPPSGQCSSAVTMRINAPVHLVWSIVRRFEEPHIFQPFVRGCTMRGSTSLAVGCVREVDFKSGFSAKSSVERLEILDDKEHVFGVRIIGGDHRLKNYSSVLTAKPEVIDGEPATLVSESFVIDVPEGNTADETRHFVEFLIRCNLRSLAMVSQRLLLAQGDLAEPPAQ

SEQ ID NO: 79 - protein, 215 aa, Oryza sativa
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGLGKVRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 80 - protein, 186 aa, Rheum rhaponticum
MNGDGYGGSEEEFVKRYHEHVLADHQCSSVLVEHINAPLHLVWSLVRSFDQPQKYKPFVSRCVVQGGDLEIGSVREVDVKSGLPATTSMEELELLDDKEHVLRVKFVGGDHRLKNYSSIVSLHPEIIGGRSGTMVIESFIVDIADGNTKEETCYFIESLINCNLKSLSCVSERLAVEDIAERIAQM

SEQ ID NO: 81 - protein, 254 aa, Oryza sativa
MVGLVGGGGWRVGDDAAGGGGGGAVAAGAAAAAEAEHMRRLHSQGPRRAPVQLRARQAHQGSCSPPRIECANFAVFLAARDPKIVWSLVRSFDQPQRYKPFVSRCVVRGGDLEIGSVREVNVKTGLPATTSTERLELLDDDEHILSVKFVGGDHRLRNYSSIVTVHPESIDGRPGTLVIESFVVDVPDGNTKDETCYFVEAVIKCNLTSLAEMVRMISLVLPFMLVDRMSGITCESHLETTLVRCGEYAVLAHV

SEQ ID NO: 82 - protein, 186 aa, Oryza sativa
MEPHMERALREAVASEAERRELEGVVRAHHTGWNAPLAAVWPHRARVRPTRSGTSTSSSRASSPPGDGATVGSVREVAVVSGLPASTSTERLEILDDDRHVLSFRVVGGDHRLRNYRSVTSVTEFSSPSSPPRPYCVVVESYVVDVPEGNTEEDTRMFTDTVVKLNLQKLAAVATSSSPPAAGNHH

SEQ ID NO: 83 - protein, 150 aa, Oryza sativa
MEVVWSIVRRFEEPHIFQPFVRGCTMRGSTSLAVGCVREVDFKSGFPAKSSVERLEILDDKEHVFGVRIIGGDHRLKNYSSVLTAKPEVIDGEPATLVSESFVVDVPEGNTADETRHFVEFLIRCNLRSLAMVSQRLLLAQGDLAEPPGQ

SEQ ID NO: 84 - protein, 206 aa, Oryza sativa
MPYTAPRPSPPQHSRIGGCGGGGVLKAAGAAGHAASCVAVPAEVARHHEHAAGVGQCCSAVVQAIAAPVDAVWRTSTSSGAAASWTATATAGPLPVGSVREFRVLSGLPGTSSRERLEILDDERRVLSFRVVGGEHRLSNYRSVTTVHETAAGAAAAVVVESYVVDVPHGNTADETRMFVDTIVRCNLQSLARTAEQLALAAPRAA

SEQ ID NO: 85 - protein, 396 aa, Vitis vinifera
  misc_feature (61)..(61): Xaa can be any naturally occurring amino acid
MPISSLPFSLYTVTPNPLKLITTHAHAFTPHTHIFTLKFMSHTYCPHIHHITSIHYTHLLXPIPHMPLQPPLPPHPILPSMPAFQHLYSTNQHLQVALFSARGPNIRDFNFQDADLLKLDILAPGSLIWAAWSPNGTDEANYVGEGSPTVAMIAKRGPRHGKYMAFCXMYRDNVAPKGVNXAVATVKTKRTIQLKTSLEIACHYAGINISGINGEVMPGQWEYQVGPGQCSSLLAQRVHVPLSAVGSVVHRFDKPQRYQHVIKSCRIEDGFEMRMGXLRDVNIISGLPTATNTGRLDMQDDERHVTRCPHQRQSESKYTENNNSDASSIKSPINGPSEHLKTAASPKTESIIVIDTSKFLNEEDFEGKDETSSSNQVQIEDENWETRFPNTDAGIW

SEQ ID NO: 86 - protein, 443 aa, Vitis vinifera
  misc_feature (5)..(5): Xaa can be any naturally occurring amino acid
MPSAXKSSTVPLSLXQFKLGLRHGHRVIPWGDLDSLAMLQRQLDVDILVTGHTHRFTAYKHEGGVVINPGSATGAFGSITYDVNPSFVLMDIDGLRVVVCVYELIDETANIIKELHARKISFGTKSMIXCLLLKRRSTPKFRRKKLFLFQCRVQMTLTLTNLAVSGIAQTLQVDQWTVCALIFMTRRDIHLDKARFLDFKDMGKLLADASGLRKALSGGXVTAGMAIFDTMRHIRPDVPTVCVGLAAVAMIAKRGPRHGKYMAFCPMYRDNVAPKGVNVAVVTVKTKRTIQLKTSLEIACHYAGINISGINGEVMPGQWEYQVGPGQCSSLLAQRVHVPLSAVGSVVHRFDKPQRYQHVIKSCRIEDGFEMRMGRLRDVNIISGLPTATNTGRLDMQDDEXHVTRCPHQRQSESKYTENNNSDASSVKSPINGPSEHLKTAAX

SEQ ID NO: 87 - protein, 95 aa, Oryza sativa
EIGSVREVNVKTGLPATTSTERLELLDDDEHILSVKFVGGDHRLRNYSSIVTVHPESIDGRPGTLVIESFVVDVPDGNTKDETCYFVEAVIKCNL

SEQ ID NO: 88 - protein, 191 aa, Zea mays
MVVEMDGGVGVAAAGGGGAQTPAPPPPRRWRLADERCDLRAMETDYVRRFHRHEPRDHQCSSAVAKHIKAPVHLVWSLVRRFDQPQLFKPFVSRCEMKGNIEIGSVREVNVKSGLPATRSTERLELLDDDERILSVRFVGGDHRLQVCSVLHLSIFCAAHARYFAHHLKCVLEFLCQMHLDVLPCDDAILE

SEQ ID NO: 89 - protein, 239 aa, Oryza sativa
MNGCTGGAGGVAAGRLPAVSLQQAQWKLVDERCELREEEMEYVRRFHRHEIGSNQCNSFIAKHVRAPLQNVWSLVRRFDQPQIYKPFVRKCVMRGNVETGSVREIIVQSGLPATRSIERLEFLDDNEYILRVKFIGGDHMLKKRIPKKTYAISSRTCSDSAIIAVGQSNCAPEITAMNGGVSIQPWLILLAFFSSPSNQTNPDSLRDMHPGSWFQILLVLAMFTCSKGSVLPPSEKVNV

SEQ ID NO: 90 - protein, 188 aa, Zea mays
MEPHMESALRQGLSEAEQRELEGVVRAHHTFPGRAPGTCTSLVTQRVDAPLAAVWPIVRGFGSPQRYKHFIKSCDLKAGDGATVGSVREVTVVSGLPASTSTERLEILDDHRHILSFRVVGGDHRLRNYRSVTSVTEFQPGPYCVVLESYVVDVPDGNTEEDTRMFTDTVVKLNLQKLAAIATSSSAN

SEQ ID NO: 91 - protein, 205 aa, Zea mays
MDQQGAGGDVEVPAGLGLTAAEYEQLRPTVDAHHRYAVGEGQCSSLLAQRIHAPPAAVWAIVRRFDCPQVYKHFIRSCAVRPDPDAGDALRPGRLREVCVISGLPASTSTERLDHLDDAARVFGFSITGGEHRLRNYRSVTTVSELAGPGICTVVLESYAVDVPDGNTEDDTRLFADTVIRLNLQKLKSVAEASTSSSAPPPPSE

SEQ ID NO: 92 - protein, 220 aa, Zea mays
MPCIQASSPGGMPHQHGRGRVLGGGVGCAAEVAAAVAASAGGMRCGAHDGEVPAEAARHHEHAAAGPGRCCSAVVQHVAAPAAAVWSVVRRFDQPQVYKRFVRSCALLAGDGGVGTLREVRVVSGLPAASSRERLEVLDDESHVLSFRVVGGEHRLRNYLSVTTVHPSPAAPDAATVVVESYVVDVPPGNTPEDTRVFVDTIVKCNLQSLATTAEKLAAV

SEQ ID NO: 93 - protein, 221 aa, Glycine max
MEKAESSASTSEPDSDENHHRHPTNHHINPPSGLTPLEFASLIPSVAEHHSYLVGSGQCSSLLAQRVQAPPDAVWSVVRRFDKPQTYKHFIKSCAVKEPFHMAVGVTRDVNVISGLPAATSTERLDLLDDIRCVTGFSIIGGEHRLRNYRSVTTVHSFEDDADDGKIYTVVLESYVVDVPDGNTEEDTRLFADTVVKLNLQKLASVTEGTNRDGDGKSHSR

SEQ ID NO: 94 - protein, 214 aa, Glycine max
MEKTHSSSAEEQDPTRRHLDPPPGLTAEEFEDLKPSVLEHHTYSVTPTRQSSSLLAQRIHAPPHAVWSVVRCFDNPQAYKHFIKSCHVKEGFQLAVGSTRDVHVISGLPAATSTERLDLLDDDRHVIGFTIVGGDHRLRNYRSVTSVHGFECDGKIWTVVLESYVVDVPEGNTEEDTRLFADTVVKLNLQKLASVSEGMCGDGDGDGDGKGNKS

SEQ ID NO: 95 - protein, 216 aa, Artificial Sequence
  synthetic soybean Glyma01g31320.1 protein
MLQNSSMSSLLLHRINGGGGATTATNCHDTVFMTVPDGVARYHTHAVAPNQCCSSVAQEIGASVATVWSVLRRFDNPQAYKHFVKSCHVIGGDGDVGTLREVHVISGLPAARSTERLEILDDERHVISFSVVGGDHRLANYRSVTTLHPTASSASGGCSGTVVVESYVVDVPPGNTREDTRVFVDTIVKCNLQSLAQTAENLTLRKNNNNDYKCCS

SEQ ID NO: 96 - protein, 208 aa, Artificial Sequence
  synthetic soybean Glyma02g42990.1 protein
MTSLQFHRFNPATDTSTAIANGVNCPKPPSTLRLLAKVSLSVPETVARHHAHPVGPNQCCSVVIQAIDAPVSAVWPVVRRFDNPQAYKHFVKSCHVVAAAGGGEDGIRVGALREVRVVSGLPAVSSTERLEILDDERHVMSFSVVGGDHRLRNYRSVTTLHGDGNGGTVVIESYVVDVPPGNTKEETCVFVDTIVRCNLQSLAQIAET

SEQ ID NO: 97 - protein, 176 aa, Glycine max
AYPVLGLTPEEFSELESIINTHHKFEPSPEICSSIIAQRIDAPAHTVWPLVRSFENPQKYKHFVKSCNMRSGDGGVGSIREVTVVSGLPASTSTERLEILDDDKHLLSFRVVGGEHRLHNYRSVTSVNEFKNPDNGKVYTIVLESYVVDIPEGNTGVDTKMFVDTVVKLNLQKLGE

SEQ ID NO: 98 - protein, 172 aa, Glycine max
EFTELESTINTHHKFEASPEICSSIIAQRIDAPAHTVWPLVRSFENPQKYKHFVKSCNMRSGDGGVGSIREVTVVSGLPASTSTERLEILDDDNHLLSFRVVGGEHRLHNYRSVTSVNEFKRPDNGKVYTIVLESYVVDIPEGNTGVDTKMFVDTVVKLNLQKLGEVAMATN

SEQ ID NO: 99 - protein, 191 aa, Glycine max
MTELSSREVEYIRRHHSKAAEDNQCASALVKHIRAPLPLVWSLVRRFDEPQKYKPFVSRCVVRGNLEIGSLREVDVKSGLPATTSTERLEILDDNHHILSVRIIGGDHRLRNYSSIMSLHPEIVDGRPGTLVIESFVVDIPEGNTKDETCYFVEALIKCNLKSLADVSEGLTLQDHTEPIDRKYELLITRG

SEQ ID NO: 100 - protein, 185 aa, Glycine max
MNGGESYGAIETQYIRRHHKHEPRENQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIDGRPGTMVIESFVVDVPDGNTRDETCYFVEALIRCNLSSLADVSERMAVQGRTNPINH

SEQ ID NO: 101 - protein, 178 aa, Glycine max
MSPNNPSTIVSDAVARHHTHVVSPHQCCSAVVQEIAAPVSTVWSVVRRFDNPQAYKHFVKSCHVILGDGDVGTLREVRVISGLPAAVSTERLDVLDDERHVIGFSMVGGDHRLSNYRSVTILHPRSATDTVVVESYVVDVPAGNTTEDTRVFVDTILRCNLQSLAKFAENLTNKLHQR

SEQ ID NO: 102 - protein, 246 aa, Glycine max
MSRSHNKRKPFSFIFKITLLELLSSLLSSSLRFAMDKTHSGEEQDPNPTHPTRNHLDPPPGLTPEEFEDLKPSVLEHHTYSVTPTRQCSSLLAQRIHAPPHTVWTVVRCFDNPQAYKHFIKSCHVKEGFQLAVGSTRDVHVISGLPAATSTERLDLLDDDRHVIGFTIVGGDHRLRNYRSVTSVHGFERDGKIWTVVLESYVVDVPEGNTEEDTRLFADTVVKLNLQKLASVTEGMCGDSDGKGNN

SEQ ID NO: 103 - protein, 223 aa, Glycine max
MEKAESSASTSEPDSDDNHHRHPTNHHLNPPSGLTPLEFASLVPSVAEHHSYLVGPGQCSSLLAQRVHAPPDAVWSFVRRFDKPQTYKHFIKSCAVKEPFHMAVGVTRDVNVISGLPAATSTERLDFLDDVRRVTGFSIIGGEHRLRNYRSVTTVHSFDDDNASADGKIYTVVLESYVVDVPDGNTEEDTRLFADTVVKLNLQKLASVTEGTNGDGDGKPHSR

SEQ ID NO: 104 - protein, 229 aa, Glycine max
MPSSLHFDRFNPITHAATTVAIANGVNCPKQPQAPPSSTAARRLVVPSLSSGRGIAAPDTVALHHAHVVDPNQCCSIVTQHINAPVSAVWAVVRRFDNPQGYKNFVRSCHVITGDGIRVGAVREVRVVSGLPAETSTERLEILDDERHVISFSMVGGDHRLRNYQSVTTLHANGNGTLVIESYVVDVPQGNTKEETCVFVDTIVRCNLQSLAQIAENRTNNCEHTAQHC

SEQ ID NO: 105 - protein, 191 aa, Glycine max
MNGIGNDGGGGLSNVEMEYIRRHHRHEPGENQCGSALVKHIRAPVPQVWSLVRRFDQPQKYKPFVSRCVVRGNLEIGSLREVDVKSGLPATTSTERLELLDDNEHLLSIRIIGGDHRLRNYSSIMSLHPEIIDGRPGTLVIESFVVDVPEGNTKDETCYFVEALIKCNLKSLADVSEGIAVQDRTEPIDRI

SEQ ID NO: 106 - protein, 169 aa, Glycine max
MVARHHAHAVGPNQCCSFVIQAIDAPVSAVWPVVRRFDNPQAYKHFVKSCHVVAAGGAGGDGGIHVGALREVRVVSGLPAVSSTERLEILDDERHVMSFSVVGGDHRLRNYRSVTTLHGDGSNGGTVVIESYVVDIPAGNTKEETCVFVDTIVRCNLQSLAQMAENMGS

SEQ ID NO: 107 - protein, 210 aa, Glycine max
MTILPHSNNKSSNHKFIAHQNYMASETHHHVQGLTPEELTKLEPIIKKYHLFEQSPNTCFSIITYRIEAPAKAVWPFVRSFDNPQKYKHFIKGCNMRGDGGVGSIREVTVVSGLPASTSTERLEILDDDKHVLSFRVVGGEHRLKNYRSVTSVNEFNKEGKVYTIVLESYIVDIPEGNTEEDTKMFVDTVVKLNLQKLGVVAMASSMHGQ

SEQ ID NO: 108 - protein, 193 aa, Artificial Sequence
  synthetic soybean Glyma14g30260.1 protein
MNRIGNGGGGGGGLSNVEMEYIRRHHRHEPGENQCGSALVKHIRAPVPQVWSLVRRFDQPQKYKPFISRCVVRGNLEIGSLREVDVKSGLPATTSTERLELLDDNEHILSIRIIGGDHRLRNYSSIMSLHPEIIDGRPGTLVIESFVVDVPEGNTKDETCYFVEALIKCNLKSLADVSEGLAVQDCTEPIDRI

SEQ ID NO: 109 - protein, 188 aa, Glycine max
MASETHHHVQGLTPEELTQLEPIIKKYHLFEASSNKCFSIITHRIEAPASSVWPLVRNFDNPQKYKHFIKGCNMKGDGSVGSIREVTVVSGLPASTSTERLEILDDDKHVLSFRVVGGEHRLQNYRSVTSVNEFHKEGKVYTIVLESYIVDIPEGNTEEDTKMFVDTVVKLNLQKLGVVAMASSMNGR

SEQ ID NO: 110 - protein, 177 aa, Glycine max
MLPNNPSTIVPDAVARHHTHVVSPQQCCSAVVQEIAAPVSTVWSVVRRFDNPQAYKHFVKSCHVILGDGDVGTLREVHVISGLPAAVSTERLDVLDDERHVIGFSMVGGDHRLFNYRSVTTLHPRSAAGTVVVESYVVDVPPGNTTEDTRVFVDTILRCNLQSLAKFAENLTKLHQR

SEQ ID NO: 111 - protein, 185 aa, Glycine max; identical to SEQ ID NO: 100

SEQ ID NO: 112 - protein, 191 aa, Glycine max
MGITIGIQCLEIEEISICDGMFCYLVDFVDVKEKMNYCLMWFGYFPSQVWSLVRRFDQPQKYKPFVSRCIMQGDLGIGSVREVNVKSGLPATTSTERLEQLDDEEHILGIRIVGGDHRLRNYSSIITVHPEVIDGRPSTMVIESFVVDVPDGNTRDETCYFVEALIRCNLSSLADVSERMAVQGRTDPINH

SEQ ID NO: 113 - protein, 185 aa, Artificial Sequence
  synthetic PYR/PYL receptor protein
MNGGESYGAIETQYIRRHHKHEPRENQCTSALVKHIRAPVHLVWSLVRRFDQPQKYKPFVSRCIMQGDLGIGSVREVNVKSGLPATTSTERLEQLDDEEHILGIRIVGGDHRLRNYSSIITVHPEVIDGRPSTMVIESFVVDVPDGNTRDETCYFVEALIRCNLSSLADVSERMAVQGRTDPINH

SEQ ID NO: 114 - protein, 204 aa, Sorghum bicolor
METHVERALRATLTEAEVRALEPAVREHHTFPAGRVAAGTTTPTPTTCTSLVAQRVSAPVRAVWPIVRSFGNPQRYKHFVRTCALAAGDGASVGSVREVTVVSGLPASSSTERLEVLDDDRHILSFRVVGGDHRLRNYRSVTSVTEFQPGPYCVVVESYAVDVPEGNTAEDTRMFTDTVVRLNLQKLAAVAEESAAAAAAGNRR

SEQ ID NO: 115 - protein, 204 aa, Sorghum bicolor
MEPHMETALRQGGLSELEQRELEPVVRAHHTFPGRSPGTTCTSLVTQRVDAPLSAVWPIVRGFAAPQRYKHFIKSCDLRSGDGATVGSVREVTVVSGLPASTSTERLEILDDDRHILSFRVVGGDHRLRNYRSVTSVTEFHHHHQAAAGRPYCVVVESYVVDVPEGNTEEDTRMFTDTVVKLNLQKLAAIATSSAAAAASNSST

SEQ ID NO: 116 - protein, 258 aa, Sorghum bicolor
MVESPNPNSPSRPLCIKYTRAPARHFSPPLPFSSLIISANPIEPKAMDKQGAGGDVEVPAGLGLTAAEYEQLRSTVDAHHRYAVGEGQCSSLLAQRIQAPPAAVWAIVRRFDCPQVYKHFIRSCALRPDPEAGDALRPGRLREVSVISGLPASTSTERLDLLDDAARVFGFSITGGEHRLRNYRSVTTVSELADPGICTVVLESYVVDVPDGNTEDDTRLFADTVIRLNLQKLKSVAEANAAAAASFVSVVPPPEPEE

SEQ ID NO: 117 - protein, 222 aa, Sorghum bicolor
MPCLQASSSPGSMPHQHHGRVLAGVGCAAEVAAAAVAATSPAAGMRCGAHDGEVPAEAARHHEHAAPGPGRCCSAVVQHVAAPASAVWSVVRRFDQPQAYKRFVRSCALLAGDGGVGTLREVRVVSGLPAASSRERLEVLDDESHVLSFRVVGGEHRLQNYLSVTTVHPSPAAPDAATVVVESYVVDVPPGNTPEDTRVFVDTIVKCNLQSLATTAEKLAAV

SEQ ID NO: 118 - protein, 211 aa, Sorghum bicolor
MVEMDGGVGVVGGGQQTPAPRRWRLADELRCDLRAMETDYVRRFHRHEPRDHQCSSAVAKHIKAPVHLVWSLVRRFDQPQLFKPFVSRCEMKGNIEIGSVREVNVKSGLPATRSTERLELLDDNEHILSVKFVGGDHRLQNYSSILTVHPEVIDGRPGTLVIESFVVDVPDGNTKDETCYFVEALLKCNLKSLAEVSERQVIKDQTEPLDR

SEQ ID NO: 119 - protein, 216 aa, Sorghum bicolor
MPYTAPRPSPQQHSRVTGGGAKAAIVAASHGASCAAVPAEVARHHEHAARAGQCCSAVVQAIAAPVGAVWSVVRRFDRPQAYKHFIRSCRLVDDGGGGAGAGAGATVAVGSVREVRVVSGLPATSSRERLEILDDERRVLSFRVVGGEHRLANYRSVTTVHEAEAGAGGTVVVESYVVDVPPGNTADETRVFVDTIVRCNLQSLARTAERLALALA

SEQ ID NO: 120 - DNA, 20 bp, Arabidopsis thaliana
aagcggcgcgtggagagaga

SEQ ID NO: 121 - DNA, 20 bp, Arabidopsis thaliana
gctgtccatctctccgtcgc

SEQ ID NO: 122 - DNA, 21 bp, Arabidopsis thaliana
tgaagtgatcgatgcaccagg

SEQ ID NO: 123 - DNA, 22 bp, Arabidopsis thaliana
gacacgacaggaaacacctttg

SEQ ID NO: 124 - DNA, 25 bp, Arabidopsis thaliana
tatgaatcctctgccgtgagaggtg

SEQ ID NO: 125 - DNA, 24 bp, Arabidopsis thaliana
acaccactgagataatccgatcct

SEQ ID NO: 126 - DNA, 22 bp, Arabidopsis thaliana
gttgatggtgtgagtgagcagc

SEQ ID NO: 127 - DNA, 23 bp, Arabidopsis thaliana
aacccattactagctgtcccaag

SEQ ID NO: 128 - DNA, 22 bp, Arabidopsis thaliana
gacgcttttgagcatcgacact

SEQ ID NO: 129 - DNA, 23 bp, Arabidopsis thaliana
actgtttccttcgctcccgtttc

SEQ ID NO: 130 - DNA, 19 bp, Arabidopsis thaliana
ctcatgaagatccttacag

SEQ ID NO: 131 - DNA, 20 bp, Arabidopsis thaliana
ctttcaggtggtgcaacgac

SEQ ID NO: 132 - DNA, 20 bp, Glycine max
acgtcagttccgcaaaagat

SEQ ID NO: 133 - DNA, 19 bp, Glycine max
ggacccgttggtttctcac

SEQ ID NO: 134 - DNA, 23 bp, Glycine max
gggaatgggaatttgggtgagaa

SEQ ID NO: 135 - DNA, 21 bp, Glycine max
ccttctgccagggctagcatg

SEQ ID NO: 136 - DNA, 23 bp, Glycine max
cctgcggcttaatttgactcaac

SEQ ID NO: 137 - DNA, 19 bp, Glycine max
taagaacggccatgcacca

SEQ ID NO: 138 - DNA, 19 bp, Hordeum vulgare
aacacgctgggcatgggag

SEQ ID NO: 139 - DNA, 22 bp, Hordeum vulgare
cgaacgaccaaacacgactaaa

SEQ ID NO: 140 - DNA, 20 bp, Hordeum vulgare
cgggcggcgcgattgcgagc

SEQ ID NO: 141 - DNA, 20 bp, Hordeum vulgare
acggaattagggccatcacg

SEQ ID NO: 142 - DNA, 20 bp, Hordeum vulgare
tccatgatggccaagtgtga

SEQ ID NO: 143 - DNA, 21 bp, Hordeum vulgare
gacatccccacggtacatgag

SEQ ID NO: 144 - DNA, 18 bp, Zea mays
gcagcaggcaggggagaa

SEQ ID NO: 145 - DNA, 19 bp, Zea mays
gccgagcgagttcatcatc

SEQ ID NO: 146 - DNA, 23 bp, Zea mays
atgagtacggtcagcaggggcag

SEQ ID NO: 147 - DNA, 21 bp, Zea mays
ctccctcgcaggctggaactg

SEQ ID NO: 148 - DNA, 27 bp, Zea mays
tgccgatgtgcctgcgtcgtctggtgc

SEQ ID NO: 149 - DNA, 26 bp, Zea mays
tgaaagacagaacataatgagcacag

(19) United States
(12) Patent Application Publication
(10) Pub. No.: US 2016/0280651 A1
(43) Pub. Date: Sep. 29, 2016

(54) COMPOUNDS THAT INDUCE ABA RESPONSES

(71) Applicants: THE REGENTS OF THE UNIVERSITY OF CALIFORNIA, Oakland, CA (US); SYNGENTA PARTICIPATIONS AG, Basel (CH)

(72) Inventors: Sean R. CUTLER, Riverside, CA (US); Sebastian Volker WENDEBORN, Basel (CH); Pierre Joseph JUNG, Basel (CH); Mathilde Denise LACHIA, Basel (CH); Raphael DUMEUNIER, Basel (CH)

(21) Appl. No.: 14/899,089

(22) PCT Filed: Jun. 27, 2014
(86) PCT No.: PCT/US2014/044727

§ 371 Date: Dec. 16, 2015

Related U.S. Application Data
(60) Provisional application No. 61/840,967, filed on Jun. 28, 2013.

(51) Int. Cl. C07D 215/227; A01N 43/42

(52) U.S. Cl. CPC C07D 215/227; A01N 43/42

(57) ABSTRACT

The present invention provides agonist compounds that active ABA receptors, and agricultural formulations comprising the agonist compounds. The agricultural formulations are useful for inducing ABA responses in plant vegetative tissues, reducing abiotic stress in plants, and inhibiting germination of plant seeds. The compounds are also useful for inducing expression of ABA-responsive genes in cells that express endogenous or heterologous ABA receptors.

The present patent application claims benefit of priority to US Provisional Patent Applictaion No. 61/840,967, filed Jun. 28, 2013, which is incorporated by reference.

### BACKGROUND OF THE INVENTION

Abscisic acid (ABA) is a plant hormone that regulates signal transduction associated with abiotic stress responses (Cutler et al., 2010, Abscisic Acid: Emergence of a Core Signaling Network. Annual Review of Plant Biology 61:651-679). The ABA signaling pathway has been exploited to improve plant stress response and associated yield traits via numerous approaches (Yang et al., 2010). The direct application of ABA to plants improves their water use efficiency (Raedmacher et al., 1987); for this reason, the discovery of ABA agonists (Park et al., 2009; Melcher et al., 2010, Identification and mechanism of ABA receptor antagonism. Nature Structural & Molecular Biology 17(9):1102-1110) has received increasing attention, as such molecules may be beneficial for improving crop yield (Notman et al., 2009). The first synthetic ABA agonist identified was the naphthalene sulfonamide named pyrabactin (Park et al., 2009), which efficiently activates ABA signaling in seeds but has limited activity in vegetative tissues, where the most critical aspects of abiotic stress tolerance occur. Sulfonamides highly similar to pyrabactin have been disclosed as ABA agonists (see US Patent Publication No. 20130045952) and abiotic stress modulating compounds (see US Patent Publication No. 20110230350); and non-sulfonamide ABA agonists have also been described (see US Patent Publication Nos. 20130045952 and 20110271408). A complementary approach to activating the ABA pathway involves increasing a plant's sensitivity to ABA via genetic methods. For example, conditional antisense of farnesyl transferase beta subunit gene, which increases a plant's ABA sensitivity, improves yield under moderate drought in both canola and Arabidopsis (Wang et al., 2005). Thus, the manipulation of ABA signaling to improve traits contributing to yield is now well established.

It has recently been discovered that ABA elicits many of its cellular responses by binding to a soluble family of receptors called PYR/PYL proteins. PYR/PYL proteins belong to a large family of ligand-binding proteins named the START superfamily (Iyer et al., 2001); Ponting et al., 1999). These proteins contain a conserved three-dimensional architecture consisting of seven anti-parallel beta sheets, which surround a central alpha helix to form a “helix-grip” motif; together, these structural elements form a ligand-binding pocket for binding ABA or other agonists.

### BRIEF SUMMARY OF THE INVENTION

The present invention provides for small molecule ABA agonists, i.e., compounds that activate PYR/PYL proteins. In one aspect, the present invention provides for ABA agonist compounds as described herein as well as agricultural formulations comprising such compounds. In some embodiments, the compound of Formula I is provided:

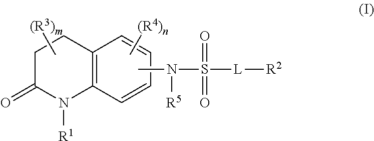

wherein
R¹ is selected from the group consisting of C₂₋₆ alkenyl, and C₂₋₆ alkynyl,
R² is selected from the group consisting of cycloalkyl, heterocycloalkyl, aryl and heteroaryl, each optionally substituted with from 1-4 R^{2a} groups,
each R^{2a} is independently selected from the group consisting of H, halogen, C₁₋₆ alkyl, C₁₋₆ alkoxy, C₁₋₆ haloalkyl, C₁₋₆ haloalkoxy, C₂₋₆ alkenyl, C₂₋₆ alkynyl, —OH, C₁₋₆ alkylhydroxy, —CN, —NO₂, —C(O)R^{2b}, —C(O)OR^{2b}, —OC(O)R^{2b}, —C(O)NR^{2b}R^{2c}, —NR^{2b}C(O)R^{2c}, —SO₂R^{2b}, —SO₂OR^{2b}, —SO₂NR^{2b}R^{2c}, and —NR^{2b}SO₂R^{2c},
each of R^{2b} and R^{2c} are independently selected from the group consisting of H and C₁₋₆ alkyl,
each of R³, R⁴ and R⁵ are independently selected from the group consisting of H and C₁₋₆ alkyl, wherein at least one R³ or R⁴ is methyl,
L is a linker selected from the group consisting of a bond and C₁₋₆ alkylene,
subscript m is an integer from 0 to 4,
subscript n is an integer from 0 to 3, and
m+n is greater than or equal to 1,
   or a salt or isomer thereof.

In some embodiments, the agricultural formulation further comprises an agricultural chemical that is useful for promoting plant growth, reducing weeds, or reducing pests. In some embodiments, the agricultural formulation further comprises at least one of a fungicide, an herbicide, a pesticide, a nematicide, an insecticide, a plant activator, a synergist, an herbicide safener, a plant growth regulator, an insect repellant, an acaricide, a molluscicide, or a fertilizer. In some embodiments, the agricultural formulation further comprises a surfactant. In some embodiments, the agricultural formulation further comprises a carrier.

In another aspect, the invention provides methods for increasing abiotic stress tolerance in a plant, the method comprising the step of contacting a plant with a sufficient amount of the above formulations to increase abiotic stress tolerance in the plant compared to the abiotic stress tolerance in the plant when not contacted with the formulation. In some embodiments, the plant is a monocot. In some embodiments, the plant is a dicot. In some embodiments, the abiotic stress tolerance comprises drought tolerance.

In another aspect, the invention provides a method of inhibiting seed germination in a plant, the method comprising the step of contacting a plant, a plant part, or a plant seed with a sufficient amount of the above formulations to inhibit germination.

In another aspect, the invention provides a plant or plant part in contact with the above formulations. In some embodiments, the plant is a seed.

In another aspect, the invention provides a method of activating a PYR/PYL protein. In some embodiments of the method, the PYR/PYL protein binds a type 2 protein phosphatase (PP2C) polypeptide when the PYR/PYL protein binds the agonist compound LC66C6 (also referred to herein as quinabactin). In some embodiments, the method comprises the step of contacting the PYR/PYL protein with any of the compounds described herein. In some embodiments, the PYR/PYL protein that is activated is substantially identical to any one of SEQ ID NOs:1-119. In some embodiments, the PYR/PYL protein is expressed by a cell. In some embodiments, the PYR/PYL protein is expressed by a plant cell. In some embodiments, the PYR/PYL protein is an endogenous protein. In some embodiments, the PYR/PYL protein is a heterologous protein. In some embodiments, the cell further expresses a type 2 protein phosphatase (PP2C). In some embodiments, the type 2 protein phosphatase is HAB1 (Homology to ABI1), ABI1 (Abscisic acid insensitive 1), or ABI2 (Abscisic acid insensitive 2).

### BRIEF DESCRIPTION OF THE DRAWINGS

FIG. 1A-FIG. 1B. Novel ABA agonists bind to multiple PYR/PYL. (A) Chemical structure of naturally occurring (+)-ABA, its (−)analog and selected ABA agonists. (B) Yeast two-hybrid agonist assays of PYR/PYL receptor sensitivity to 5 μM of test chemicals. Specific PYR/PYL Receptors and the PP2C HAB1 are expressed as Ga14 BD or AD fusion proteins respectively, as described in the text.

FIG. 2A-FIG. 2C. Novel ABA agonists inhibit PPC2 activity through multiple PYR/PYL. (A) Chemical structure of naturally occurring (+)-ABA and selected ABA agonists. (B) and (C) HAB1, ABU, and ABI2 PP2C enzyme activity based ABA-agonist assays for various receptors in the presence or absence of 10 μM each test chemical.

FIG. 3A-FIG. 3B. (A) Receptor-mediated dose-dependent inhibition of PP2C enzyme activity by ABA agonists and analogs. (B) Observed compound IC₅₀ values in enzymatic HAB1 PP2C-based ABA-agonist assays.

FIG. 4A-FIG. 4B. Quinabactin activates multiple ABA receptors. (A) Chemical structures of ABA, pyrabactin and quinabactin. (B) Chemical-dependent inhibition of HAB1 by ABA receptors. IC₅₀ values (nM) were determined as described in the methods using 50 nM HAB1, 50 nM and multiple concentrations of compounds; full dose response curves are provided as in FIG. 3. (nd) correspond to receptors that were not produced as active proteins. The phylogenetic tree is a Neighbor-Joining tree made using the JTT distance matrix in MEGA5 (Tamura K, et al. (2011) MEGA5: Molecular Evolutionary Genetics Analysis Using Maximum Likelihood, Evolutionary Distance, and Maximum Parsimony Methods. Molecular Biology and Evolution 28(10):2731-2739).

FIG. 5A-FIG. 5D. Novel ABA agonists inhibit germination of Arabidopsis seeds more strongly than pyrabactin. (A) and (B) Comparison of seed germination inhibition by ABA agonists. (C) and (D) the effects of ABA and LC66C6 (also called quinabactin) on Arabidopsis ABA signaling- and biosynthesis-deficient mutants on germination (C) and seedling establishment (D). Seeds were sown on ½×MS agar plate containing chemicals, and were stored at 4° C. for 4 days, then transferred at 22±2° C. Photographs (A and C) and germination (B) or green cotyledon (D) scores were assessed after a 4-day incubation under continuous illumination. Panel C shows germination assays on 5 μM of ABA or LC66C6.

FIG. 6A-FIG. 6C. LC66C6 inhibits plant growth. (A) Photographs showing the effect of ABA, Pyrabactin and LC66C6 on the wild type, abi1-1 and PYR/PYL quadruple mutant Arabidopsis genotypes. (B) Root growth inhibition and (C) plant growth inhibition by ABA, LC66C6 and pyrabactin. Two day old seedlings were transferred on ½×MS plate containing chemicals and phenotypes scored or photographed after a 5-day incubation on test compounds.

FIG. 7A-FIG. 7E. LC66C6 enhances drought stress tolerance. LC66C6 represses the transpirational water loss of detached leaves in wild type (A) and the aba2 mutant genotypes (B). (C) LC66C6 cannot rescue the phenotypes of the ABA-insensitive genotype abi1-1. (D) LC66C6 induces stomatal closure in the wild type and aba2, but not abi1-1 genotypes. (E) Effects of compounds on soil water content during drought treatments in soybean. Soil water content was measured as described in the examples.

FIG. 8A-FIG. 8B. Quinabactin confers drought stress tolerance to wild-type plants. (A) Effect of quinabactin on Arabidopsis drought tolerance. Two-week-old plants were subjected to drought stress by withholding water and were photographed after 12 days. During the drought period, plants were treated every 3 days with 25 μM compound. Plants were re-hydrated after 2 weeks drought treatment; the number of surviving plants (out of total number tested) for each treatment is shown next to each image. (B) Effects of quinabactin on soybean. Two-week-old plants were subjected to drought stress by withholding water and photographed after 8 days drought treatment. For all drought stress treatments, compounds (tested at 25 μM for Arabidopsis and 50 μM for soybean) were applied in solutions containing 0.05% Tween-20 and applied as aerosols every 3 days over the drought regime. Values for all experiments are means±SEM (n=6, 3 plants used per experiment).

FIG. 9A-FIG. 9D. LC66C6 induces numerous ABA-responsive genes. (A) Shows the chemical induced mRNA expression levels of the ABA-responsive reporter genes RD29B and MAPKKK18 in wild-type, abi1-1, the pyr1/pyl1/pyl2/pyl4 quadruple receptor mutant genotypes of Arabidopsis seedlings treated with either vehicle (DMSO), pyrabactin, LC66C6, or (+)-ABA. (B) LC66C6 efficiently induces ABA-responsive genes in Arabidopsis seedlings, while pyrabactin does not. Ten-day old seedlings were treated with carrier solvent (DMSO) or either 25 μM ABA, pyrabactin or LC66C6 for 8 hours. Total RNA was then prepared labeled and hybridized to ATH1 microarrays. Data plotted are log 2 transformed average expression values for ˜13K probes that were detectable across all experiments. Data shown are averages determined from triplicate biological replicates. (C) and (D) show the expression of a reporter gene in different plant tissues after treatment with vehicle (DMSO), pyrabactin, LC66C6, or (+)-ABA.

FIG. 10. ABA-responsive gene expression in PYR/PYL single mutants. The response of the ABA-responsive MAPKKK18, RD29A, and RD29B mRNAs to LC66C6, ABA and pyrabactin were characterized in the Col and Ler ecotypes and the pyr1, pyl1, ply2, pyl3 and pyl4 single mutant genotypes.

FIG. 11. LC66C6 induces ABA-responsive gene expression in wild-type plants, abi1-1 and PYR/PYL quadruple mutants. LC66C6 and (+)-ABA induced expression of ABF3, GBF3, NCED3, and RD29A in a dose dependent manner in Col wild-type plants, while pyrabactin does not.

FIG. 12A-FIG. 12B. LC66C6 sensitivity is not influenced by the CYP707A ABA-hydroxylating enzymes. (A) shows photographs and (B) shows quantitation of primary root length in wild-type plants, plants that overexpress CYP707A (CYP707AOX), and plants that are double mutant for cyp707a treated with DMSO, 40 μM (+)-ABA, and 40 μM LC66C6. (C) shows fresh weight and (D) shows the percent of plants with green cotyledons in the plants treated as in (A).

FIG. 13A-FIG. 13E. LC66C6 modulates ABA responses in diverse species. Germination inhibition (A) and transpirational water loss in detached leaves 2-hours after detachment (B) in response to compounds shown. The expression of ABA-responsive marker genes in Soybean (C), Barley (D) and Maize (E) after application of chemicals. D, P, L and A indicate DMSO, pyrabactin, LC66C6 and (+)-ABA, respectively.

FIG. 14. Chemical structure of ABA and agonists.

FIG. 15A-FIG. 15C. The effect of ABA and agonists in yeast assays and seed germination. (A) shows the results of yeast two-hybrid assays using PYR/PYL receptors PYR1, PYL1, PYL2, PYL3, and PYL4 to test the response to each of the agonists shown in FIG. 14. (B) shows the results of testing the agonists in FIG. 14 on germination of wild-type seeds. (C) shows effects of compounds on an ABA-reporter line as measured using glucuronidase assays in a transgenic line expressing glucuronidase under the control of the ABA-inducible Arabidopsis gene MAPKKK18.

FIG. 16A-FIG. 16B. Application of LC66C6 can rescue growth defects observed in the ABA-deficient mutant aba2. Chemical solution (25 μM) was sprayed on 14-day-plants two times per day for 2 weeks. The image (A) and fresh weight (B) were obtained from 4-week plants.

FIG. 17A-FIG. 17D. The effect of ABA and its agonists in Physcomitrella patens and Chlamydomonas. Protonemal growth images (A) and quantitative analysis (B) of the effects of ABA and agonists on Phsycomitrella patens. Protonema were grown on 200 μM of specific test chemical for 10 days. LC66C6's effects were weak, but significantly inhibited protonema growth. Pyrabactin bleached protonema. (C) The expression of ABA-responsive genes of Physcomitrella patens. Protonema were treated with 200 μM chemical solutions for 3 h. (D) Colony growth of Chlamydomonas on the chemical with salinity stress and osmotic stress. There was no effect of ABA and LC66C6 on the Chlamydomonas growth with and without stresses. Pryabactin bleached Physcomitrella patens and Chlamydomonas, suggesting that this compound may have toxicity in these species unrelated to its ABA agonist activity.

FIG. 18 shows a summary of the agonist compounds tested for their effect on inhibition of germination and pMAPKK18:Gus reporter expression. ++++++ indicates strong activity, whereas a single + indicates weak activity, a dash (−) indicates no activity, and n.d. indicates not determined.

### DEFINITIONS

“Agonists” are agents that, e.g., induce or activate the expression of a described target protein or bind to, stimulate, increase, open, activate, facilitate, enhance activation, sensitize or up-regulate the activity of one or more plant PYR/PYL proteins (or encoding polynucleotide). Agonists can include naturally occurring and synthetic molecules. In some embodiments, the agonists are combined with agrichemicals to produce and agricultural formulation. Examples of suitable agrichemicals include fungicides, herbicides, pesticides, fertilizers, and/or surfactants. Assays for determining whether an agonist “agonizes” or “does not agonize” a PYR/PYL protein include, e.g., contacting putative agonists to purified PYR/PYL protein(s) and then determining the functional effects on the PYR/PYL protein activity, as described herein, or contacting putative agonists to cells expressing PYR/PYL protein(s) and then determining the functional effects on the described target protein activity, as described herein. One of skill in the art will be able to determine whether an assay is suitable for determining whether an agonist agonizes or does not agonize a PYR/PYL protein. Samples or assays comprising PYR/PYL proteins that are treated with a putative agonist are compared to control samples without the agonist to examine the extent of effect. Control samples (untreated with agonists) are assigned a relative activity value of 100%. Agonism of the PYR/PYL protein is achieved when the activity value relative to the control is 110%, optionally 150%, optionally 200, 300%, 400%, 500%, or 1000-3000% or more higher.

The term “PYR/PYL receptor polypeptide” refers to a protein characterized in part by the presence of one or more or all of a polyketide cyclase domain 2 (PF10604), a polyketide cyclase domain 1 (PF03364), and a Bet V I domain (PF03364), which in wild-type form mediates abscisic acid (ABA) and ABA analog signaling. A wide variety of PYR/PYL receptor polypeptide sequences are known in the art. In some embodiments, a PYR/PYL receptor polypeptide comprises a polypeptide that is substantially identical to any one of SEQ ID NOs:1-119. See, e.g., Published PCT Application WO 2011/139798.

The term “activity assay” refers to any assay that measures or detects the activity of a PYR/PYL receptor polypeptide. An exemplary assay to measure PYR/PYL receptor activity is a yeast two-hybrid assay that detects binding of a PYR/PYL polypeptide to a type 2 protein phosphatase (PP2C) polypeptide, as described in the Examples.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” used in the context of two nucleic acids or polypeptides, refers to a sequence that has at least 60% sequence identity with a reference sequence. Alternatively, percent identity can be any integer from 60% to 100%. Some embodiments include at least: 60%, 65%, 70%, 75%, 80%, 85%, 90%, 91%, 92%, 93%, 94% 95%, 96%, 97%, 98%, or 99%, compared to a reference sequence using the programs described herein; preferably BLAST using standard parameters, as described below. Embodiments of the present invention provide for polypeptides, and nucleic acids encoding polypeptides, that are substantially identical to any of SEQ ID NO:1-119.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

(see, e.g., Creighton, Proteins (1984)).

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the methods of the invention includes angiosperms (monocotyledonous and dicotyledonous plants), gymnosperms, ferns, bryophytes, and multicellular and unicellular algae. It includes plants of a variety of ploidy levels, including aneuploid, polyploid, diploid, haploid, and hemizygous.

As used herein, the term “transgenic” describes a non-naturally occurring plant that contains a genome modified by man, wherein the plant includes in its genome an exogenous nucleic acid molecule, which can be derived from the same or a different plant species. The exogenous nucleic acid molecule can be a gene regulatory element such as a promoter, enhancer, or other regulatory element, or can contain a coding sequence, which can be linked to a heterologous gene regulatory element. Transgenic plants that arise from sexual cross or by selfing are descendants of such a plant and are also considered “transgenic.”.

As used herein, the term “drought-resistance” or “drought-tolerance,” including any of their variations, refers to the ability of a plant to recover from periods of drought stress (i.e., little or no water for a period of days). Typically, the drought stress will be at least 5 days and can be as long as, for example, 18 to 20 days or more (e.g., at least 6, 7, 8, 9, 10, 11, 12, 13, 14, 15, 16, 17, 18, 19, 20 days), depending on, for example, the plant species.

As used herein, the terms “abiotic stress,” “stress,” or “stress condition” refer to the exposure of a plant, plant cell, or the like, to a non-living (“abiotic”) physical or chemical agent that has an adverse effect on metabolism, growth, development, propagation, or survival of the plant (collectively, “growth”). A stress can be imposed on a plant due, for example, to an environmental factor such as water (e.g., flooding, drought, or dehydration), anaerobic conditions (e.g., a lower level of oxygen or high level of CO₂), abnormal osmotic conditions, salinity, or temperature (e.g., hot/heat, cold, freezing, or frost), a deficiency of nutrients or exposure to pollutants, or by a hormone, second messenger, or other molecule. Anaerobic stress, for example, is due to a reduction in oxygen levels (hypoxia or anoxia) sufficient to produce a stress response. A flooding stress can be due to prolonged or transient immersion of a plant, plant part, tissue, or isolated cell in a liquid medium such as occurs during monsoon, wet season, flash flooding, or excessive irrigation of plants, or the like. A cold stress or heat stress can occur due to a decrease or increase, respectively, in the temperature from the optimum range of growth temperatures for a particular plant species. Such optimum growth temperature ranges are readily determined or known to those skilled in the art. Dehydration stress can be induced by the loss of water, reduced turgor, or reduced water content of a cell, tissue, organ or whole plant. Drought stress can be induced by or associated with the deprivation of water or reduced supply of water to a cell, tissue, organ or organism. Salinity-induced stress (salt-stress) can be associated with or induced by a perturbation in the osmotic potential of the intracellular or extracellular environment of a cell. As used herein, the term “abiotic stress tolerance” or “stress tolerance” refers to a plant's increased resistance or tolerance to abiotic stress as compared to plants under normal conditions and the ability to perform in a relatively superior manner when under abiotic stress conditions.

A polypeptide sequence is “heterologous” to an organism or a second polypeptide sequence if it originates from a foreign species, or, if from the same species, is modified from its original form.

### DETAILED DESCRIPTION OF THE INVENTION

### I. Introduction

The present invention is based, in part, on the discovery of selective abscisic acid (ABA) agonists. Unlike previous ABA agonists, the agonists described herein potently activate the ABA pathway in plant vegetative tissues and induce abiotic stress tolerance. The new agonists can be used to induce stress tolerance in crop species of plants. The agonists can be used to induce stress tolerance in monocot and dicot plant species, including but not limited to broccoli, radish, alfalfa, soybean, barley, and corn (maize).

Abscisic acid is a multifunctional phytohormone involved in a variety of phyto-protective functions including bud dormancy, seed dormancy and/or maturation, abscission of leaves and fruits, and response to a wide variety of biological stresses (e.g. cold, heat, salinity, and drought). ABA is also responsible for regulating stomatal closure by a mechanism independent of CO₂ concentration. The PYR/PYL family of ABA receptor proteins mediate ABA signaling. Plants examined to date express more than one PYR/PYL receptor protein family member, which have at least somewhat redundant activity. PYR/PYL receptor proteins mediate ABA signaling as a positive regulator in, for example, seed germination, post-germination growth, stomatal movement and plant tolerance to stress including, but not limited to, drought.

A wide variety of wild-type (naturally occurring) PYR/PYL polypeptide sequences are known in the art. Although PYR1 was originally identified as an abscisic acid (ABA) receptor in Arabidopsis, in fact PYR1 is a member of a group of at least 14 proteins (PYR/PYL proteins) in the same protein family in Arabidopsis that also mediate ABA signaling. This protein family is also present in other plants (see, e.g., SEQUENCE LISTING) and is characterized in part by the presence of one or more or all of a polyketide cyclase domain 2 (PF10604), a polyketide cyclase domain 1 (PF03364), and a Bet V I domain (PF03364). START/Bet v 1 superfamily domain are described in, for example, Radauer, BMC Evol. Biol. 8:286 (2008). In some embodiments, a wild-type PYR/PYL receptor polypeptide comprises any of SEQ ID NOs:1-119. In some embodiments, a wild-type PYR/PYL receptor polypeptide is substantially identical to (e.g., at least 70%, 75%, 80%, 85%, 90%, 91%, 92%, 93%, 94% 95%, 96%, 97%, 98%, or 99% identical to) any of SEQ ID NOs:1-119. In some embodiments, a PYR/PYL receptor polypeptide is substantially identical to (e.g., at least 70%, 75%, 80%, 85%, 90%, 91%, 92%, 93%, 94% 95%, 96%, 97%, 98%, or 99% identical to) any of SEQ ID NO:1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, or 119.

### II. ABA Agonists

The present invention provides for small molecule ABA agonists, i.e., compounds that activate PYR/PYL proteins. Exemplary ABA agonists include, e.g., a compound selected from the following:

A compound of Formula (I):

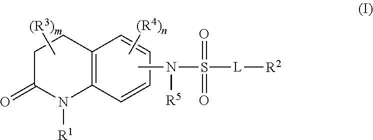

wherein
R¹ is selected from the group consisting of C₂₋₆ alkenyl, and C₂₋₆ alkynyl,
R² is selected from the group consisting of cycloalkyl, heterocycloalkyl, aryl and heteroaryl, each optionally substituted with from 1-4 R^{2a} groups,
each R^{2a} is independently selected from the group consisting of H, halogen, C₁₋₆ alkyl, C₁₋₆ alkoxy, C₁₋₆ haloalkyl, C₁₋₆ haloalkoxy, C₂₋₆ alkenyl, C₂₋₆ alkynyl, —OH, C₁₋₆ alkylhydroxy, —CN, —NO₂, —C(O)R^{2b}, —C(O)OR^{2b}, —OC(O)R^{2b}, —C(O)NR^{2b}R^{2c}, —NR^{2b}C(O)R^{2c}, —SO₂R^{2b}, —SO₂OR^{2b}, —SO₂NR^{2b}R^{2c}, and —NR^{2b}SO₂R^{2c},
each of R^{2b} and R² are independently selected from the group consisting of H and C₁₋₆ alkyl,
each of R³, R⁴ and R⁵ are independently selected from the group consisting of H and

C₁₋₆ alkyl, wherein at least one R³ or R⁴ is methyl,
L is a linker selected from the group consisting of a bond and C₁₋₆ alkylene,
subscript m is an integer from 0 to 4,
subscript n is an integer from 0 to 3, and
m+n is greater than or equal to 1,
   or a salt or isomer thereof.

In some embodiments, L is CH₂. In some embodiments, R³ is CH₃. In some embodiments, R³ is CH₃ and R⁴ is H. In some embodiments, R³ is H and R⁴ is CH₃. In some embodiments, R⁵ is H. In some embodiments, m is 2 and both R³ groups are CH₃.

In some embodiments, the compound of Formula (I) has the formula (I-A):

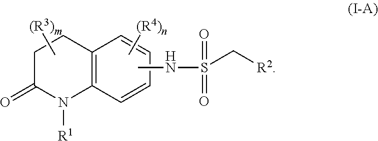

In some embodiments, the compound of Formula (I) has the formula (I-B):

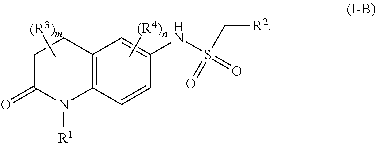

In some embodiments, R² is selected from the group consisting of aryl and heteroaryl, each optionally substituted with from 1-4 R^{2a} groups.

In some embodiments, each R^{2a} is independently selected from the group consisting of H, halogen and C₁₋₆ alkyl.

In some embodiments, R² is selected from the group consisting of phenyl, naphthyl, thiophene, furan, pyrrole, and pyridyl.

In some embodiments, R² is selected from the group consisting of phenyl and thiophene, each optionally substituted with 1 R^{2a} group; each R^{2a} is independently selected from the group consisting of H, F, Cl, methyl, and ethyl; and L is selected from the group consisting of a bond and methylene.

In some embodiments, the compound of Formula (I) has the formula (I-C):

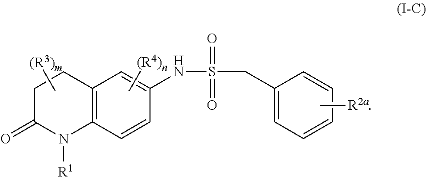

In some embodiments, the compound of Formula (I) has the formula (I-D):

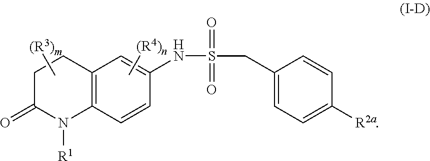

In some embodiments, m is 4 and n is 3. Optionally, the compound of Formula I where m is 4 and n is 3 can be represented by the compound of Formula I-E as shown below.

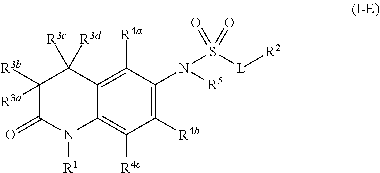

In Formula I-E, R^{3d}, R^{3b}, R^{3c}, and R^{3d} are each independently defined as in R³ for Formula I. Also in Formula I-E, R^{4a}, R^{4b}, and R^{4b} are each independently defined as in R⁴ for Formula I.

In some embodiments, Formula I-E can be represented as one of Structures 1 through 59 as shown below:

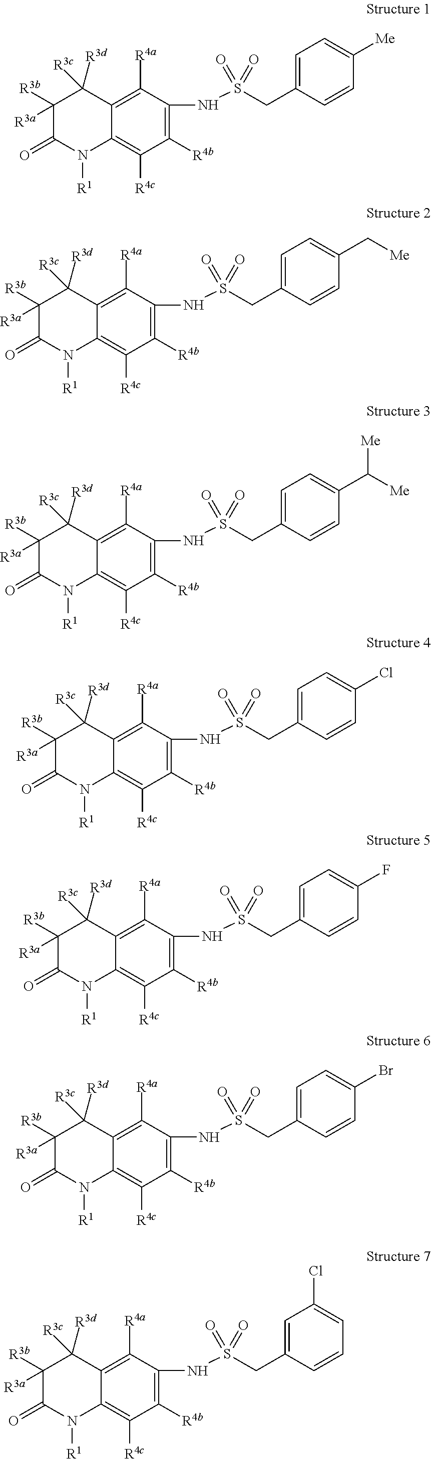

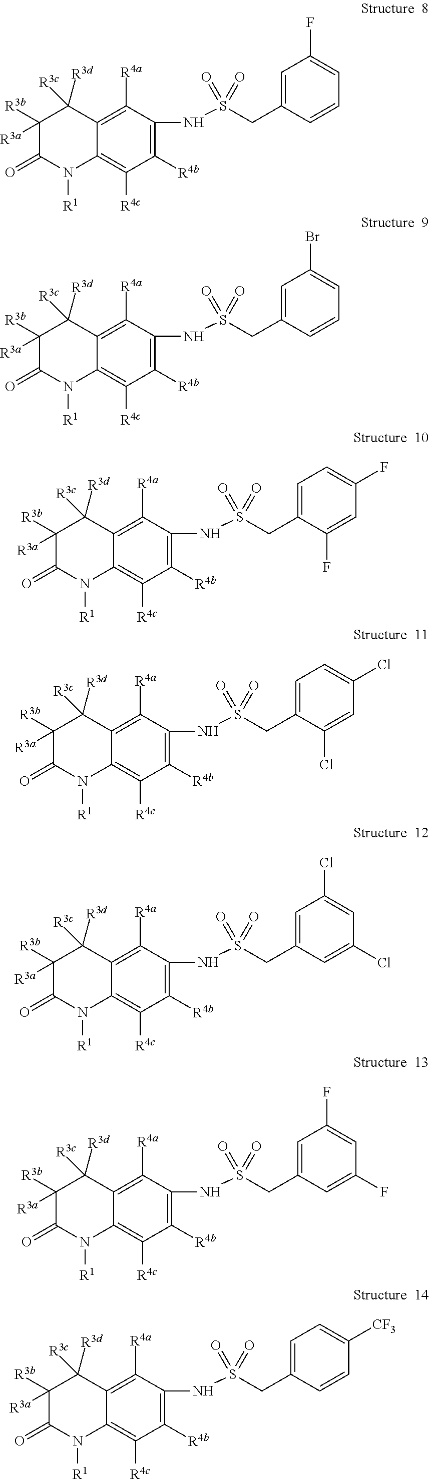

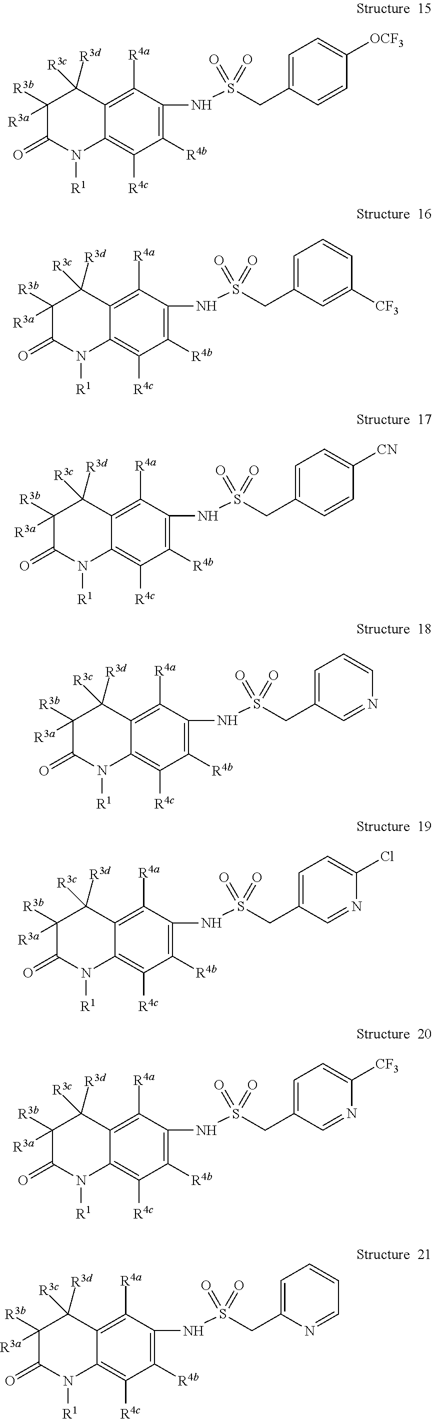

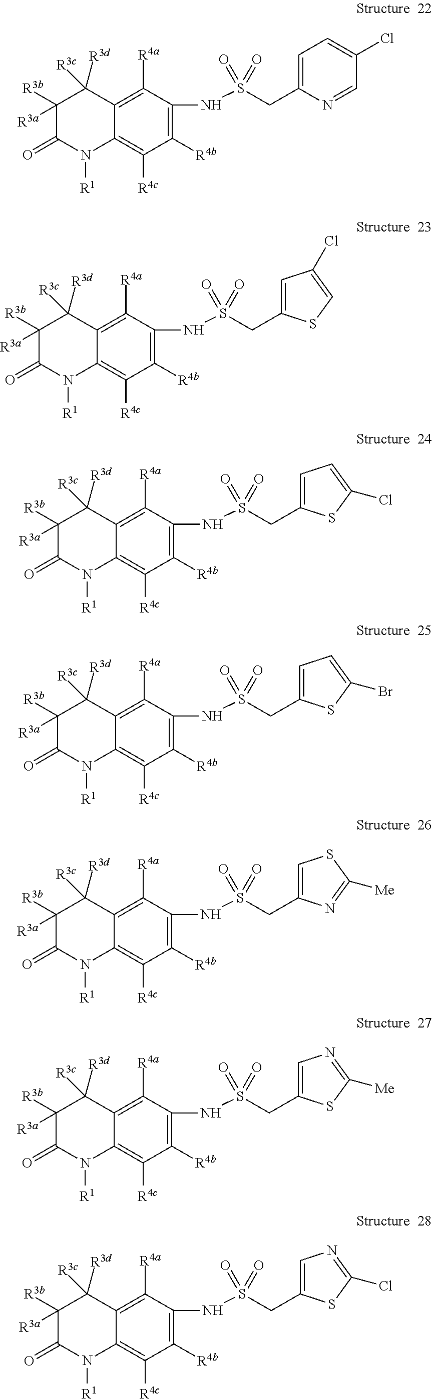

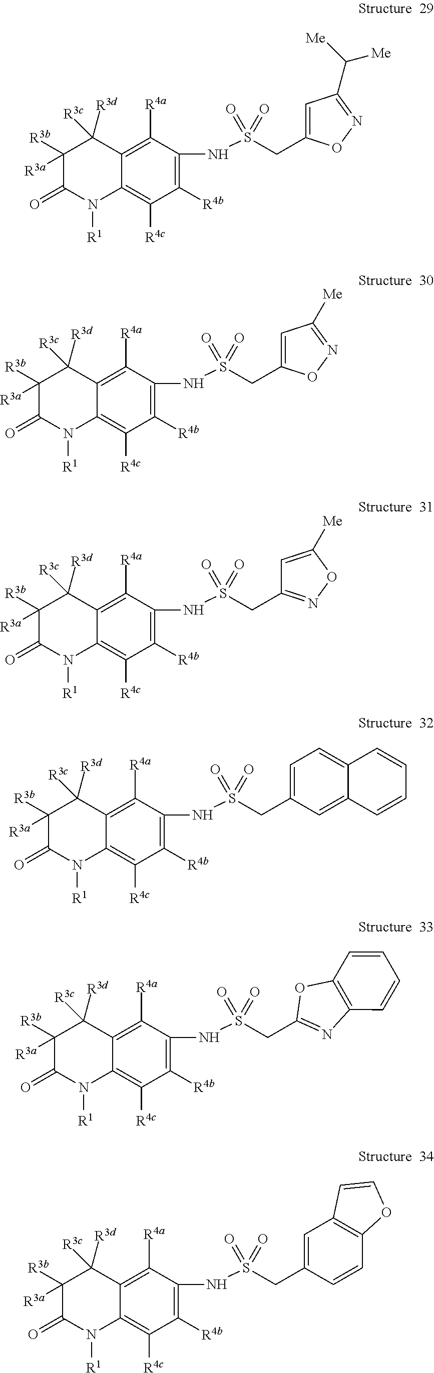

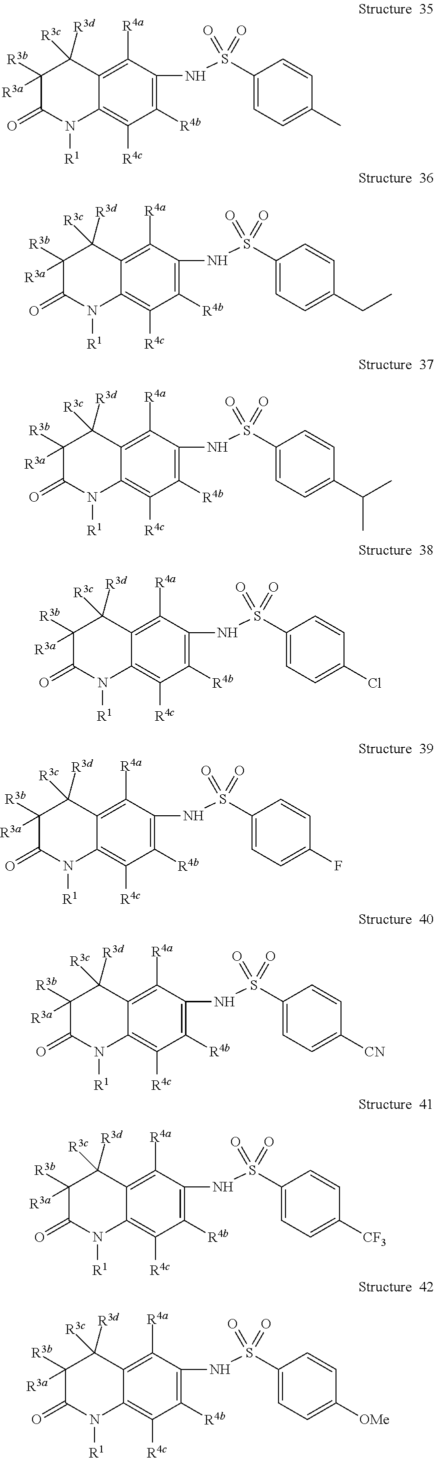

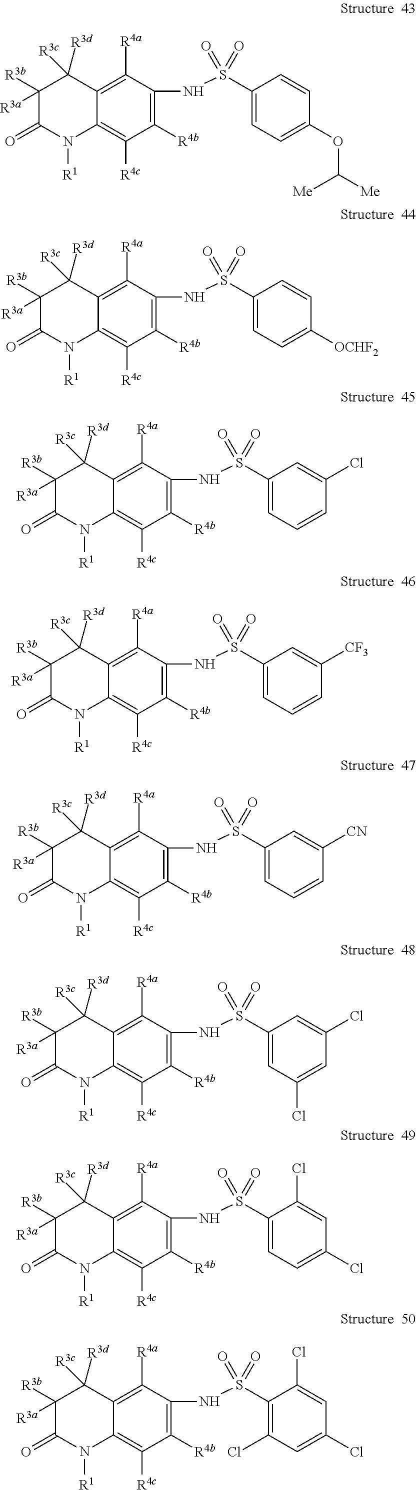

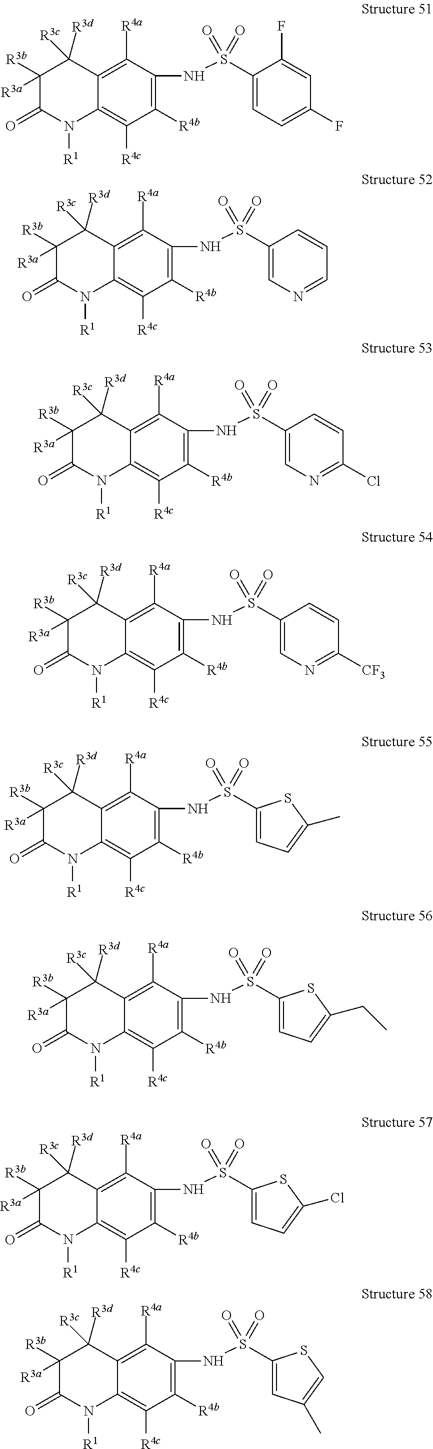

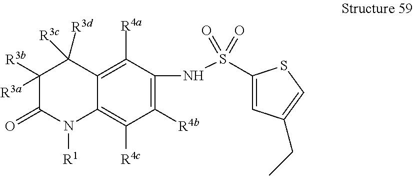

Exemplary compounds according to Structure 1, Structure 2, Structure 3, Structure 4, Structure 5, Structure 6, Structure 7, Structure 8, Structure 9, Structure 10, Structure 11, Structure 12, Structure 13, Structure 14, Structure 15, Structure 16, Structure 17, Structure 18, Structure 19, Structure 20, Structure 21, Structure 22, Structure 23, Structure 24, Structure 25, Structure 26, Structure 27, Structure 28, Structure 29, Structure 30, Structure 31, Structure 32, Structure 33, Structure 34, Structure 35, Structure 36, Structure 37, Structure 38, Structure 39, Structure 40, Structure 41, Structure 42, Structure 43, Structure 44, Structure 45, Structure 46, Structure 47, Structure 48, Structure 49, Structure 50, Structure 51, Structure 52, Structure 53, Structure 54, Structure 55, Structure 56, Structure 57, Structure 58, and Structure 59 are shown below in Table 1. In Table 1, substituents R¹, R^{3a}, R^{3b}, R^{3c}, R^{3d}, R^{4a}, R^{4b}, and R^{4b} are listed for each compound. Each combination of substituents listed in Table 1 can be used in each of Structures 1 through 59.

For reference purposes, each individual compound is identified according to the structure number and the substituent identification shown in Table 1. For example, the compound of Structure 1 where R¹ is CH₂CH═CH₂, R^{3a} is methyl, and R^{3b}, R^{3c}, R^{3d}, R^{4a}, R^{4b}, and R^{4c} are each H is labeled as Compound 1.001. In another example, the compound of Structure 24 where R¹ is CH₂CH═CHCH₃ (E) and R^{3a}, R^{3b}, R^{3c}, R^{3d}, R^{4a}, R^{4b}, and R^{4c} are each H is labeled as Compound 24.016.

TABLE 1
Exemplary Compounds

| Sub- | | | | | | | | |
|---|---|---|---|---|---|---|---|---|
| stitu- | | | | | | | | |
| ent | | | | | | | | |
| ID | R¹ | R^{3a} | R^{3b} | R^{3c} | R^{3d} | R^{4a} | R^{4b} | R^{4c} |
| .001 | CH₂CH═CH₂ | Me | H | H | H | H | H | H |
| .002 | CH₂CH═CH₂ | Me | Me | H | H | H | H | H |
| .003 | CH₂CH═CH₂ | H | H | Me | H | H | H | H |
| .004 | CH₂CH═CH₂ | H | H | Me | Me | H | H | H |
| .005 | CH₂CH═CH₂ | H | H | H | H | Me | H | H |
| .006 | CH₂CH═CH₂ | H | H | H | H | H | Me | H |
| .007 | CH₂CH═CH₂ | H | H | H | H | H | H | Me |
| .008 | CH₂CH═CH₂ | H | H | H | H | H | H | H |
| .009 | CH₂CH═CHCH₃ (E) | Me | H | H | H | H | H | H |
| .010 | CH₂CH═CHCH₃ (E) | Me | Me | H | H | H | H | H |
| .011 | CH₂CH═CHCH₃ (E) | H | H | Me | H | H | H | H |
| .012 | CH₂CH═CHCH₃ (E) | H | H | Me | Me | H | H | H |
| .013 | CH₂CH═CHCH₃ (E) | H | H | H | H | Me | H | H |
| .014 | CH₂CH═CHCH₃ (E) | H | H | H | H | H | Me | H |
| .015 | CH₂CH═CHCH₃ (E) | H | H | H | H | H | H | Me |
| .016 | CH₂CH═CHCH₃ (E) | H | H | H | H | H | H | H |
| .017 | CH₂CH═CHCH₃ (Z) | Me | H | H | H | H | H | H |
| .018 | CH₂CH═CHCH₃ (Z) | Me | Me | H | H | H | H | H |
| .019 | CH₂CH═CHCH₃ (Z) | H | H | Me | H | H | H | H |
| .020 | CH₂CH═CHCH₃ (Z) | H | H | Me | Me | H | H | H |
| .021 | CH₂CH═CHCH₃ (Z) | H | H | H | H | Me | H | H |
| .022 | CH₂CH═CHCH₃ (Z) | H | H | H | H | H | Me | H |
| .023 | CH₂CH═CHCH₃ (Z) | H | H | H | H | H | H | Me |
| .024 | CH₂CH═CHCH₃ (Z) | H | H | H | H | H | H | H |
| .025 | CH₂CH═C(CH₃)₂ | Me | H | H | H | H | H | H |
| .026 | CH₂CH═C(CH₃)₂ | Me | Me | H | H | H | H | H |
| .027 | CH₂CH═C(CH₃)₂ | H | H | Me | H | H | H | H |
| .028 | CH₂CH═C(CH₃)₂ | H | H | Me | Me | H | H | H |
| .029 | CH₂CH═C(CH₃)₂ | H | H | H | H | Me | H | H |
| .030 | CH₂CH═C(CH₃)₂ | H | H | H | H | H | Me | H |
| .031 | CH₂CH═C(CH₃)₂ | H | H | H | H | H | H | Me |
| .032 | CH₂CH═C(CH₃)₂ | H | H | H | H | H | H | H |
| .033 | CH₂C(CH₃)═CH₂ | Me | H | H | H | H | H | H |
| .034 | CH₂C(CH₃)═CH₂ | Me | Me | H | H | H | H | H |
| .035 | CH₂C(CH₃)═CH₂ | H | H | Me | H | H | H | H |
| .036 | CH₂C(CH₃)═CH₂ | H | H | Me | Me | H | H | H |
| .037 | CH₂C(CH₃)═CH₂ | H | H | H | H | Me | H | H |
| .038 | CH₂C(CH₃)═CH₂ | H | H | H | H | H | Me | H |
| .039 | CH₂C(CH₃)═CH₂ | H | H | H | H | H | H | Me |
| .040 | CH₂C(CH₃)═CH₂ | H | H | H | H | H | H | H |
| .041 | CH₂CH₂CH═CH₂ | Me | H | H | H | H | H | H |
| .042 | CH₂CH₂CH═CH₂ | Me | Me | H | H | H | H | H |
| .043 | CH₂CH₂CH═CH₂ | H | H | Me | H | H | H | H |
| .044 | CH₂CH₂CH═CH₂ | H | H | Me | Me | H | H | H |
| .045 | CH₂CH₂CH═CH₂ | H | H | H | H | Me | H | H |
| .046 | CH₂CH₂CH═CH₂ | H | H | H | H | H | Me | H |
| .047 | CH₂CH₂CH═CH₂ | H | H | H | H | H | H | Me |
| .048 | CH₂CH₂CH═CH₂ | H | H | H | H | H | H | H |
| .049 | CH₂CH₂CH═CHCH₃ | Me | H | H | H | H | H | H |
| | (E) | | | | | | | |
| .050 | CH₂CH₂CH═CHCH₃ | Me | Me | H | H | H | H | H |
| | (E) | | | | | | | |
| .051 | CH₂CH₂CH═CHCH₃ | H | H | Me | H | H | H | H |
| | (E) | | | | | | | |
| .052 | CH₂CH₂CH═CHCH₃ | H | H | Me | Me | H | H | H |
| | (E) | | | | | | | |
| .053 | CH₂CH₂CH═CHCH₃ | H | H | H | H | Me | H | H |
| | (E) | | | | | | | |
| .054 | CH₂CH₂CH═CHCH₃ | H | H | H | H | H | Me | H |
| | (E) | | | | | | | |
| .055 | CH₂CH₂CH═CHCH₃ | H | H | H | H | H | H | Me |
| | (E) | | | | | | | |
| .056 | CH₂CH₂CH═CHCH₃ | H | H | H | H | H | H | H |
| | (E) | | | | | | | |
| .057 | CH₂CH₂CH═CHCH₃ | Me | H | H | H | H | H | H |
| | (Z) | | | | | | | |
| .058 | CH₂CH₂CH═CHCH₃ | Me | Me | H | H | H | H | H |
| | (Z) | | | | | | | |
| .059 | CH₂CH₂CH═CHCH₃ | H | H | Me | H | H | H | H |
| | (Z) | | | | | | | |
| .060 | CH₂CH₂CH═CHCH₃ | H | H | Me | Me | H | H | H |
| | (Z) | | | | | | | |
| .061 | CH₂CH₂CH═CHCH₃ | H | H | H | H | Me | H | H |
| | (Z) | | | | | | | |
| .062 | CH₂CH₂CH═CHCH₃ | H | H | H | H | H | Me | H |
| | (Z) | | | | | | | |
| .063 | CH₂CH₂CH═CHCH₃ | H | H | H | H | H | H | Me |
| | (Z) | | | | | | | |
| .064 | CH₂CH₂CH═CHCH₃ | H | H | H | H | H | H | H |
| | (Z) | | | | | | | |
| .065 | CH₂CH₂CH═C(CH₃)₂ | Me | H | H | H | H | H | H |
| .066 | CH₂CH₂CH═C(CH₃)₂ | Me | Me | H | H | H | H | H |
| .067 | CH₂CH₂CH═C(CH₃)₂ | H | H | Me | H | H | H | H |
| .068 | CH₂CH₂CH═C(CH₃)₂ | H | H | Me | Me | H | H | H |
| .069 | CH₂CH₂CH═C(CH₃)₂ | H | H | H | H | Me | H | H |
| .070 | CH₂CH₂CH═C(CH₃)₂ | H | H | H | H | H | Me | H |
| .071 | CH₂CH₂CH═C(CH₃)₂ | H | H | H | H | H | H | Me |
| .072 | CH₂CH₂CH═C(CH₃)₂ | H | H | H | H | H | H | H |
| .073 | CH₂C≡CH | Me | H | H | H | H | H | H |
| .074 | CH₂C≡CH | Me | Me | H | H | H | H | H |
| .075 | CH₂C≡CH | H | H | Me | H | H | H | H |
| .076 | CH₂C≡CH | H | H | Me | Me | H | H | H |
| .077 | CH₂C≡CH | H | H | H | H | Me | H | H |
| .078 | CH₂C≡CH | H | H | H | H | H | Me | H |
| .079 | CH₂C≡CH | H | H | H | H | H | H | Me |
| .080 | CH₂C≡CH | H | H | H | H | H | H | H |
| .081 | CH₂C≡CMe | Me | H | H | H | H | H | H |
| .082 | CH₂C≡CMe | Me | Me | H | H | H | H | H |
| .083 | CH₂C≡CMe | H | H | Me | H | H | H | H |
| .084 | CH₂C≡CMe | H | H | Me | Me | H | H | H |
| .085 | CH₂C≡CMe | H | H | H | H | Me | H | H |
| .086 | CH₂C≡CMe | H | H | H | H | H | Me | H |
| .087 | CH₂C≡CMe | H | H | H | H | H | H | Me |
| .088 | CH₂C≡CMe | H | H | H | H | H | H | H |
| .089 | CH₂CH₂C≡CH | Me | H | H | H | H | H | H |
| .090 | CH₂CH₂C≡CH | Me | Me | H | H | H | H | H |
| .091 | CH₂CH₂C≡CH | H | H | Me | H | H | H | H |
| .092 | CH₂CH₂C≡CH | H | H | Me | Me | H | H | H |
| .093 | CH₂CH₂C≡CH | H | H | H | H | Me | H | H |
| .094 | CH₂CH₂C≡CH | H | H | H | H | H | Me | H |
| .095 | CH₂CH₂C≡CH | H | H | H | H | H | H | Me |
| .096 | CH₂CH₂C≡CH | H | H | H | H | H | H | H |
| .097 | CH₂CH₂C≡CMe | Me | H | H | H | H | H | H |
| .098 | CH₂CH₂C≡CMe | Me | Me | H | H | H | H | H |
| .099 | CH₂CH₂C≡CMe | H | H | Me | H | H | H | H |
| .100 | CH₂CH₂C≡CMe | H | H | Me | Me | H | H | H |
| .101 | CH₂CH₂C≡CMe | H | H | H | H | Me | H | H |
| .102 | CH₂CH₂C≡CMe | H | H | H | H | H | Me | H |
| .103 | CH₂CH₂C≡CMe | H | H | H | H | H | H | Me |
| .104 | CH₂CH₂C≡CMe | H | H | H | H | H | H | H |
| .105 | CH₂CH₃ | Me | H | H | H | H | H | H |
| .106 | CH₂CH₃ | Me | Me | H | H | H | H | H |
| .107 | CH₂CH₃ | H | H | Me | H | H | H | H |
| .108 | CH₂CH₃ | H | H | Me | Me | H | H | H |
| .109 | CH₂CH₃ | H | H | H | H | Me | H | H |
| .110 | CH₂CH₃ | H | H | H | H | H | Me | H |
| .111 | CH₂CH₃ | H | H | H | H | H | H | Me |
| .112 | CH₂CH₃ | H | H | H | H | H | H | H |
| .113 | CH₂CH₂CH₃ | Me | H | H | H | H | H | H |
| .114 | CH₂CH₂CH₃ | Me | Me | H | H | H | H | H |
| .115 | CH₂CH₂CH₃ | H | H | Me | H | H | H | H |
| .116 | CH₂CH₂CH₃ | H | H | Me | Me | H | H | H |
| .117 | CH₂CH₂CH₃ | H | H | H | H | Me | H | H |
| .118 | CH₂CH₂CH₃ | H | H | H | H | H | Me | H |
| .119 | CH₂CH₂CH₃ | H | H | H | H | H | H | Me |
| .120 | CH₂CH₂CH₃ | H | H | H | H | H | H | H |
| .121 | CH₂CH₂CH₂CH₃ | Me | H | H | H | H | H | H |
| .122 | CH₂CH₂CH₂CH₃ | Me | Me | H | H | H | H | H |
| .123 | CH₂CH₂CH₂CH₃ | H | H | Me | H | H | H | H |
| .124 | CH₂CH₂CH₂CH₃ | H | H | Me | Me | H | H | H |
| .125 | CH₂CH₂CH₂CH₃ | H | H | H | H | Me | H | H |
| .126 | CH₂CH₂CH₂CH₃ | H | H | H | H | H | Me | H |
| .127 | CH₂CH₂CH₂CH₃ | H | H | H | H | H | H | Me |
| .128 | CH₂CH₂CH₂CH₃ | H | H | H | H | H | H | H |
| .129 | CH₂CH═CH₂ | Et | H | H | H | H | H | H |
| .130 | CH₂CH═CH₂ | Et | Et | H | H | H | H | H |
| .131 | CH₂CH═CH₂ | H | H | Et | H | H | H | H |
| .132 | CH₂CH═CH₂ | H | H | Et | Et | H | H | H |
| .133 | CH₂CH═CH₂ | H | H | H | H | Et | H | H |
| .134 | CH₂CH═CH₂ | H | H | H | H | H | Et | H |
| .135 | CH₂CH═CH₂ | H | H | H | H | H | H | Et |
| .136 | CH₂CH═CHCH₃ (E) | Et | H | H | H | H | H | H |
| .137 | CH₂CH═CHCH₃ (E) | Et | Et | H | H | H | H | H |
| .138 | CH₂CH═CHCH₃ (E) | H | H | Et | H | H | H | H |
| .139 | CH₂CH═CHCH₃ (E) | H | H | Et | Et | H | H | H |
| .140 | CH₂CH═CHCH₃ (E) | H | H | H | H | Et | H | H |
| .141 | CH₂CH═CHCH₃ (E) | H | H | H | H | H | Et | H |
| .142 | CH₂CH═CHCH₃ (E) | H | H | H | H | H | H | Et |
| .143 | CH₂CH═CHCH₃ (Z) | Et | H | H | H | H | H | H |
| .144 | CH₂CH═CHCH₃ (Z) | Et | Et | H | H | H | H | H |
| .145 | CH₂CH═CHCH₃ (Z) | H | H | Et | H | H | H | H |
| .146 | CH₂CH═CHCH₃ (Z) | H | H | Et | Et | H | H | H |
| .147 | CH₂CH═CHCH₃ (Z) | H | H | H | H | Et | H | H |
| .148 | CH₂CH═CHCH₃ (Z) | H | H | H | H | H | Et | H |
| .149 | CH₂CH═CHCH₃ (Z) | H | H | H | H | H | H | Et |
| .150 | CH₂CH═C(CH₃)₂ | Et | H | H | H | H | H | H |
| .151 | CH₂CH═C(CH₃)₂ | Et | Et | H | H | H | H | H |
| .152 | CH₂CH═C(CH₃)₂ | H | H | Et | H | H | H | H |
| .153 | CH₂CH═C(CH₃)₂ | H | H | Et | Et | H | H | H |
| .154 | CH₂CH═C(CH₃)₂ | H | H | H | H | Et | H | H |
| .155 | CH₂CH═C(CH₃)₂ | H | H | H | H | H | Et | H |
| .156 | CH₂CH═C(CH₃)₂ | H | H | H | H | H | H | Et |
| .157 | CH₂C(CH₃)═CH₂ | Et | H | H | H | H | H | H |
| .158 | CH₂C(CH₃)═CH₂ | Et | Et | H | H | H | H | H |
| .159 | CH₂C(CH₃)═CH₂ | H | H | Et | H | H | H | H |
| .160 | CH₂C(CH₃)═CH₂ | H | H | Et | Et | H | H | H |
| .161 | CH₂C(CH₃)═CH₂ | H | H | H | H | Et | H | H |
| .162 | CH₂C(CH₃)═CH₂ | H | H | H | H | H | Et | H |
| .163 | CH₂C(CH₃)═CH₂ | H | H | H | H | H | H | Et |
| .164 | CH₂CH₂CH═CH₂ | Et | H | H | H | H | H | H |
| .165 | CH₂CH₂CH═CH₂ | Et | Et | H | H | H | H | H |
| .166 | CH₂CH₂CH═CH₂ | H | H | Et | H | H | H | H |
| .167 | CH₂CH₂CH═CH₂ | H | H | Et | Et | H | H | H |
| .168 | CH₂CH₂CH═CH₂ | H | H | H | H | Et | H | H |
| .169 | CH₂CH₂CH═CH₂ | H | H | H | H | H | Et | H |
| .170 | CH₂CH₂CH═CH₂ | H | H | H | H | H | H | Et |
| .171 | CH₂CH₂CH═CHCH₃ | Et | H | H | H | H | H | H |
| | (E) | | | | | | | |
| .172 | CH₂CH₂CH═CHCH₃ | Et | Et | H | H | H | H | H |
| | (E) | | | | | | | |
| .173 | CH₂CH₂CH═CHCH₃ | H | H | Et | H | H | H | H |
| | (E) | | | | | | | |
| .174 | CH₂CH₂CH═CHCH₃ | H | H | Et | Et | H | H | H |
| | (E) | | | | | | | |
| .175 | CH₂CH₂CH═CHCH₃ | H | H | H | H | Et | H | H |
| | (E) | | | | | | | |
| .176 | CH₂CH₂CH═CHCH₃ | H | H | H | H | H | Et | H |
| | (E) | | | | | | | |
| .177 | CH₂CH₂CH═CHCH₃ | H | H | H | H | H | H | Et |
| | (E) | | | | | | | |
| .178 | CH₂CH₂CH═CHCH₃ | Et | H | H | H | H | H | H |
| | (Z) | | | | | | | |
| .179 | CH₂CH₂CH═CHCH₃ | Et | Et | H | H | H | H | H |
| | (Z) | | | | | | | |
| .180 | CH₂CH₂CH═CHCH₃ | H | H | Et | H | H | H | H |
| | (Z) | | | | | | | |
| .181 | CH₂CH₂CH═CHCH₃ | H | H | Et | Et | H | H | H |
| | (Z) | | | | | | | |
| .182 | CH₂CH₂CH═CHCH₃ | H | H | H | H | Et | H | H |
| | (Z) | | | | | | | |
| .183 | CH₂CH₂CH═CHCH₃ | H | H | H | H | H | Et | H |
| | (Z) | | | | | | | |
| .184 | CH₂CH₂CH═CHCH₃ | H | H | H | H | H | H | Et |
| | (Z) | | | | | | | |
| .185 | CH₂CH₂CH═C(CH₃)₂ | Et | H | H | H | H | H | H |
| .186 | CH₂CH₂CH═C(CH₃)₂ | Et | Et | H | H | H | H | H |
| .187 | CH₂CH₂CH═C(CH₃)₂ | H | H | Et | H | H | H | H |
| .188 | CH₂CH₂CH═C(CH₃)₂ | H | H | Et | Et | H | H | H |
| .189 | CH₂CH₂CH═C(CH₃)₂ | H | H | H | H | Et | H | H |
| .190 | CH₂CH₂CH═C(CH₃)₂ | H | H | H | H | H | Et | H |
| .191 | CH₂CH₂CH═C(CH₃)₂ | H | H | H | H | H | H | Et |
| .192 | CH₂C≡CH | Et | H | H | H | H | H | H |
| .193 | CH₂C≡CH | Et | Et | H | H | H | H | H |
| .194 | CH₂C≡CH | H | H | Et | H | H | H | H |
| .195 | CH₂C≡CH | H | H | Et | Et | H | H | H |
| .196 | CH₂C≡CH | H | H | H | H | Et | H | H |
| .197 | CH₂C≡CH | H | H | H | H | H | Et | H |
| .198 | CH₂C≡CH | H | H | H | H | H | H | Et |
| .199 | CH₂C≡CMe | Et | H | H | H | H | H | H |
| .200 | CH₂C≡CMe | Et | Et | H | H | H | H | H |
| .201 | CH₂C≡CMe | H | H | Et | H | H | H | H |
| .202 | CH₂C≡CMe | H | H | Et | Et | H | H | H |
| .203 | CH₂C≡CMe | H | H | H | H | Et | H | H |
| .204 | CH₂C≡CMe | H | H | H | H | H | Et | H |
| .205 | CH₂C≡CMe | H | H | H | H | H | H | Et |
| .206 | CH₂CH₂C≡CH | Et | H | H | H | H | H | H |
| .207 | CH₂CH₂C≡CH | Et | Et | H | H | H | H | H |
| .208 | CH₂CH₂C≡CH | H | H | Et | H | H | H | H |
| .209 | CH₂CH₂C≡CH | H | H | Et | Et | H | H | H |
| .210 | CH₂CH₂C≡CH | H | H | H | H | Et | H | H |
| .211 | CH₂CH₂C≡CH | H | H | H | H | H | Et | H |
| .212 | CH₂CH₂C≡CH | H | H | H | H | H | H | Et |
| .213 | CH₂CH₂C≡CMe | Et | H | H | H | H | H | H |
| .214 | CH₂CH₂C≡CMe | Et | Et | H | H | H | H | H |
| .215 | CH₂CH₂C≡CMe | H | H | Et | H | H | H | H |
| .216 | CH₂CH₂C≡CMe | H | H | Et | Et | H | H | H |
| .217 | CH₂CH₂C≡CMe | H | H | H | H | Et | H | H |
| .218 | CH₂CH₂C≡CMe | H | H | H | H | H | Et | H |
| .219 | CH₂CH₂C≡CMe | H | H | H | H | H | H | Et |
| .220 | CH₂CH₃ | Et | H | H | H | H | H | H |
| .221 | CH₂CH₃ | Et | Et | H | H | H | H | H |
| .222 | CH₂CH₃ | H | H | Et | H | H | H | H |
| .223 | CH₂CH₃ | H | H | Et | Et | H | H | H |
| .224 | CH₂CH₃ | H | H | H | H | Et | H | H |
| .225 | CH₂CH₃ | H | H | H | H | H | Et | H |
| .226 | CH₂CH₃ | H | H | H | H | H | H | Et |
| .227 | CH₂CH₂CH₃ | Et | H | H | H | H | H | H |
| .228 | CH₂CH₂CH₃ | Et | Et | H | H | H | H | H |
| .229 | CH₂CH₂CH₃ | H | H | Et | H | H | H | H |
| .230 | CH₂CH₂CH₃ | H | H | Et | Et | H | H | H |
| .231 | CH₂CH₂CH₃ | H | H | H | H | Et | H | H |
| .232 | CH₂CH₂CH₃ | H | H | H | H | H | Et | H |
| .233 | CH₂CH₂CH₃ | H | H | H | H | H | H | Et |
| .234 | CH₂CH₂CH₂CH₃ | Et | H | H | H | H | H | H |
| .235 | CH₂CH₂CH₂CH₃ | Et | Et | H | H | H | H | H |
| .236 | CH₂CH₂CH₂CH₃ | H | H | Et | H | H | H | H |
| .237 | CH₂CH₂CH₂CH₃ | H | H | Et | Et | H | H | H |
| .238 | CH₂CH₂CH₂CH₃ | H | H | H | H | Et | H | H |
| .239 | CH₂CH₂CH₂CH₃ | H | H | H | H | H | Et | H |
| .240 | CH₂CH₂CH₂CH₃ | H | H | H | H | H | H | Et |

In some embodiments, the compound is one of Structures 1 through 59 having a combination of substituents as shown in Table 1.

Further exemplary ABA agonists include, e.g., a compound selected from the following:

A compound of Formula II:

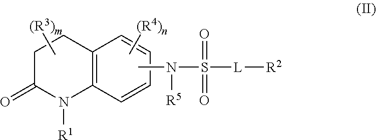

wherein
R¹ is selected from the group consisting of n-propyl,
R² is selected from the group consisting of cycloalkyl, heterocycloalkyl, aryl and heteroaryl, each optionally substituted with from 1-4 R^{2a} groups,
each R^{2a} is independently selected from the group consisting of H, halogen, C₁₋₆ alkyl, C₁₋₆ alkoxy, C₁₋₆ haloalkyl, C₁₋₆ haloalkoxy, C₂₋₆ alkenyl, C₂₋₆ alkynyl, —OH, C₁₋₆ alkylhydroxy, —CN, —NO₂, —C(O)R^{2b}, —C(O)OR^{2b}, —OC(O)R^{2b}, —C(O)NR^{2b}R^{2c}, —NR^{2b}C(O)R^{2c}, —SO₂R^{2b}, —SO₂OR^{2b}, —SO₂NR^{2b}R^{2c}, and —NR^{2b}SO₂R^{2c},
each of R^{2b} and R^{2c} are independently selected from the group consisting of H and C₁₋₆ alkyl,
each of R³, R⁴ and R⁵ are independently selected from the group consisting of H and C₁₋₆ alkyl, wherein at least one R³ or R⁴ is methyl,
L is a linker selected from the group consisting of a bond and C₁₋₆ alkylene,
subscript m is an integer from 0 to 4,
subscript n is an integer from 0 to 3, and
m+n is greater than or equal to 1,
   or a salt or isomer thereof.

A compound of Formula III:

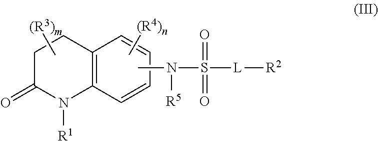

wherein
R¹ is selected from the group consisting of C₂₋₆ alkenyl, and C₂₋₆ alkynyl,
R² is selected from the group consisting of cycloalkyl, heterocycloalkyl, aryl and heteroaryl, each optionally substituted with from 1-4 R^{2a} groups,
each R^{2a} is independently selected from the group consisting of H, halogen, C₁₋₆ alkyl, C₁₋₆ alkoxy, C₁₋₆ haloalkyl, C₁₋₆ haloalkoxy, C₂₋₆ alkenyl, C₂₋₆ alkynyl, —OH, C₁₋₆ alkylhydroxy, —CN, —NO₂, —C(O)R^{2b}, —C(O)OR^{2b}, —OC(O)R^{2b}, —C(O)NR^{2b}R^{2c}, —NR^{2b}C(O)R^{2c}, —SO₂R^{2b}, —SO₂OR^{2b}, —SO₂NR^{2b}R^{2c}, and —NR^{2b}SO₂R^{2c},
each of R^{2b} and R^{2c} are independently selected from the group consisting of H and C₁₋₆ alkyl,
each of R³, R⁴ and R⁵ are independently selected from the group consisting of H and C₁₋₆ alkyl, wherein at least one R³ or R⁴ is alkyl,
L is a linker selected from the group consisting of a bond and C₁₋₆ alkylene,
subscript m is an integer from 0 to 4,
subscript n is an integer from 0 to 3, and
m+n is greater than or equal to 1,
   or a salt or isomer thereof.

In one embodiment, the at least one R³ or R⁴ is ethyl.

The compounds described above can be synthesized using methods well known in the art. For example, compounds based on the same chemical scaffold were synthesized as described in U.S. Pat. No. 5,498,755 and U.S. Pat. No. 6,127,382, the contents of which are incorporated herein by reference in their entirety.

### III. ABA Agonist Formulations

The present invention provides for agricultural chemical formulations formulated for contacting to plants, wherein the formulation comprises an ABA agonist of the present invention. In some embodiments, the plants that are contacted with the agonists comprise or express an endogenous PYR/PYL polypeptide. In some embodiments, the plants that are contacted with the agonists do not comprise or express a heterologous PYR/PYL polypeptide (e.g., the plants are not transgenic or are transgenic but express heterologous proteins other than heterologous PYR/PYL proteins). In some embodiments, the plants that are contacted with the agonists do comprise or express a heterologous PYR/PYL polypeptide as described herein.

The formulations can be suitable for treating plants or plant propagation material, such as seeds, in accordance with the present invention, e.g., in a carrier. Suitable additives include buffering agents, wetting agents, coating agents, polysaccharides, and abrading agents. Exemplary carriers include water, aqueous solutions, slurries, solids and dry powders (e.g., peat, wheat, bran, vermiculite, clay, pasteurized soil, many forms of calcium carbonate, dolomite, various grades of gypsum, bentonite and other clay minerals, rock phosphates and other phosphorous compounds, titanium dioxide, humus, talc, alginate and activated charcoal. Any agriculturally suitable carrier known to one skilled in the art would be acceptable and is contemplated for use in the present invention). Optionally, the formulations can also include at least one surfactant, herbicide, fungicide, pesticide, or fertilizer.

In some embodiments, the agricultural chemical formulation comprises at least one of a surfactant, an herbicide, a pesticide, such as but not limited to a fungicide, a bactericide, an insecticide, an acaricide, and a nematicide, a plant activator, a synergist, an herbicide safener, a plant growth regulator, an insect repellant, or a fertilizer.

In some embodiments, the agricultural chemical formulation comprises an effective amount of one or more herbicides selected from the group consisting of: paraquat (592), mesotrione (500), sulcotrione (710), clomazone (159), fentrazamide (340), mefenacet (491), oxaziclomefone (583), indanofan (450), glyphosate (407), prosulfocarb (656), molinate (542), triasulfuron (773), halosulfuron-methyl (414), pretilachlor (632), topramezone, tembotrione, isoxaflutole, fomesafen, clodinafop-propargyl, fluazifop-P-butyl, dicamba, 2,4-D, pinoxaden, bicyclopyrone, metolachlor, and pyroxasulfone. The above herbicidal active ingredients are described, for example, in “The Pesticide Manual”, Editor C. D. S. Tomlin, 12th Edition, British Crop Protection Council, 2000, under the entry numbers added in parentheses; for example, mesotrione (500) is described therein under entry number 500. The above compounds are described, for example, in U.S. Pat. No. 7,338,920, which is incorporated by reference herein in its entirety.

In some embodiments, the agricultural chemical formulation comprises an effective amount of one or more fungicides selected from the group consisting of: sedaxane, fludioxonil, penthiopyrad, prothioconazole, flutriafol, difenoconazole, azoxystrobin, captan, cyproconazole, cyprodinil, boscalid, diniconazole, epoxiconazole, fluoxastrobin, trifloxystrobin, metalaxyl, metalaxyl-M (mefenoxam), fluquinconazole, fenarimol, nuarimol, pyrifenox, pyraclostrobin, thiabendazole, tebuconazole, triadimenol, benalaxyl, benalaxyl-M, benomyl, carbendazim, carboxin, flutolanil, fuberizadole, guazatine, myclobutanil, tetraconazole, imazalil, metconazole, bitertanol, cymoxanil, ipconazole, iprodione, prochloraz, pencycuron, propamocarb, silthiofam, thiram, triazoxide, triticonazole, tolylfluanid, isopyrazam, mandipropamid, thiabendazole, fluxapyroxad, and a manganese compound (such as mancozeb, maneb). In some embodiments, the agricultural chemical formulation comprises an effective amount of one or more of an insecticide, an acaricide and/or nematcide selected from the group consisting of: thiamethoxam, imidacloprid, clothianidin, lamda-cyhalothrin, tefluthrin, beta-cyfluthrin, permethrin, abamectin, fipronil, cyanotraniliprole, chlorantraniliprole, and spinosad. Details (e.g., structure, chemical name, commercial names, etc.) of each of the above pesticides with a common name can be found in the e-Pesticide Manual, version 3.1, 13th Edition, Ed. CDC Tomlin, British Crop Protection Council, 2004-05. The above compounds are described, for example, in U.S. Pat. No. 8,124,565, which is incorporated by reference herein in its entirety.

In some embodiments, the agricultural chemical formulation comprises an effective amount of one or more fungicides selected from the group consisting of: Cyprodinil ((4-cyclopropyl-6-methyl-pyrimidin-2-yl)-phenyl-amine) (208), Dodine (289); Chlorothalonil (142); Folpet (400); Prothioconazole (685); Boscalid (88); Proquinazid (682); Dithianon (279); Fluazinam (363); Ipconazole (468); and Metrafenone. Some of the above compounds are described, for example, in “The Pesticide Manual” [The Pesticide Manual—A World Compendium; Thirteenth Edition; Editor: C. D. S. Tomlin; The British Crop Protection Council, 2003], under the entry numbers added in parentheses. The above compounds are described, for example, in U.S. Pat. No. 8,349,345, which is incorporated by reference herein in its entirety.

In some embodiments, the agricultural chemical formulation comprises an effective amount of one or more fungicides selected from the group consisting of: fludioxonil, metalaxyl and a strobilurin fungicide, or a mixture thereof. In some embodiments, the strobilurin fungicide is azoxystrobin, picoxystrobin, kresoxim-methyl, or trifloxystorbin. In some embodiments, the agricultural chemical formulation comprises an effective amount of one or more of an insecticide selected from a phenylpyrazole and a neonicotinoid. In some embodiments, the phenylpyrazole is fipronil and the neonicotinoid is selected from thiamethoxam, imidacloprid, thiacloprid, clothianidin, nitenpyram and acetamiprid. The above compounds are described, for example, in U.S. Pat. No. 7,071,188, which is incorporated by reference herein in its entirety. In some embodiments, the agricultural chemical formulation comprises an effective amount of one or more biological pesticide, including but not limited to, Pasteuria spp., Paeciliomyces, Pochonia chlamydosporia, Myrothecium metabolites, Muscodor volatiles, Tagetes spp., bacillus firmus, including bacillus firmus CNCM 1-1582.

### IV. Application to Plants

The ABA agonist formulations and compositions can be applied to plants using a variety of known methods, e.g., by spraying, atomizing, dipping, pouring, irrigating, dusting or scattering the compositions over the propagation material, or brushing or pouring or otherwise contacting the compositions over the plant or, in the event of seed, by coating, encapsulating, spraying, dipping, immersing the seed in a liquid composition, or otherwise treating the seed. In an alternative to directly treating a plant or seed before planting, the formulations of the invention can also be introduced into the soil or other media into which the seed is to be planted. For example, the formulations can be introduced into the soil by spraying, scattering, pouring, irrigating or otherwise treating the soil. In some embodiments, a carrier is also used in this embodiment. The carrier can be solid or liquid, as noted above. In some embodiments peat is suspended in water as a carrier of the ABA agonist, and this mixture is sprayed into the soil or planting media and/or over the seed as it is planted.

The types of plant that can be treated with the ABA agonists described herein include both monocotyledonous and dicotyledonous plant species including cereals such as barley, rye, sorghum, tritcale, oats, rice, wheat, soybean and corn; beets (for example sugar beet and fodder beet); cucurbits including cucumber, muskmelon, canteloupe, squash and watermelon; cale crops including broccoli, cabbage, cauliflower, bok choi, and other leafy greens, other vegetables including tomato, pepper, lettuce, beans, pea, onion, garlic and peanut; oil crops including canola, peanut, sunflower, rape, and soybean; solanaceous plants including tobacco; tuber and root crops including potato, yam, radish, beets, carrots and sweet potatoes; fruits including strawberry; fiber crops including cotton and hemp; other plants including coffee, bedding plants, perennials, woody ornamentals, turf and cut flowers including carnation and roses; sugar cane; containerized tree crops; evergreen trees including fir and pine; deciduous trees including maple and oak; and fruit and nut trees including cherry, apple, pear, almond, peach, walnut and citrus. Further types of plants that can be treated with the ABA agonists described herein include crops that are tolerant to certain chemicals, such as herbicides or fungicides. For example, genetically modified crops engineered for herbicide tolerance can be treated with the ABA agonists described herein.

It will be understood that the ABA agonists described herein mimic the function of ABA on cells. Thus, it is expected that one or more cellular responses triggered by contacting the cell with ABA will also be triggered be contacting the cell with the ABA agonists described herein. The ABA agonists described herein mimic the function of ABA and are provided in a useful formulation.

In some embodiments, application of the ABA agonists described herein increases the abiotic stress resistance of a plant.

In some embodiments, application of the ABA agonists described herein to seeds inhibits germination of the seeds.

The present invention also provides plants in contact with the ABA formulations described herein. The plant in contact with the ABA formulation can include a plant part and/or a seed.

### V. Screening for New ABA Agonists and Antagonists

Embodiments of the present invention also provide for methods of screening putative chemical agonists to determine whether the putative agonist agonizes a PYR/PYL receptor polypeptide, when the putative agonist is contacted to the PYR/PYL receptor polypeptide. As used herein, an agent “agonizes” a PYR/PYL receptor protein if the presence of the agent results in activation or up-regulation of activity of the receptor, e.g., to increase downstream signaling from the PYR/PYL receptor. For the present invention, an agent agonizes a PYR/PYL receptor if, when the agent is present at a concentration no greater than 200 μM, contacting the agent to the PYR/PYL receptor results in activation or up-regulation of the activity of the PYR/PYL receptor. If an agent does not induce activation or up-regulation of a PYR/PYL receptor protein's activity when the agent is present at a concentration no greater than 200 μM, then the agent does not significantly agonize the PYR/PYL receptor. As used herein, “activation” requires a minimum threshold of activity to be induced by the agent. Determining whether this minimum threshold of activity has been met can be accomplished, e.g., by using an enzymatic phosphatase assay that sets a minimum value for the level of enzymatic activity that must be induced, or by using an enzymatic phosphatase assay in the presence of a colorimetric detection reagent (e.g., para-nitrophenylphosphate) wherein the minimum threshold of activity has been met if a color change is observed.

The present invention also provides methods of screening for ABA agonists and antagonists by screening for a molecule's ability to induce PYR/PYL-PP2C binding in the case of agonists, or to disrupt the ability of ABA and other agonists to promote PYR/PYL-PP2C binding in the case of antagonists. A number of different screening protocols can be utilized to identify agents that agonize or antagonize a PYR/PYL polypeptide.

Screening can take place using isolated, purified or partially purified reagents. In some embodiments, purified or partially purified PYR/PYL polypeptide can be used.

Alternatively, cell-based methods of screening can be used. For example, cells that naturally-express a PYR/PYL polypeptide or that recombinantly express a PYR/PYL polypeptide can be used. In some embodiments, the cells used are plant cells, animal cells, bacterial cells, fungal cells, including but not limited to yeast cells, insect cells, or mammalian cells. In general terms, the screening methods involve screening a plurality of agents to identify an agent that modulates the activity of a PYR/PYL polypeptide by, e.g., binding to PYR/PYL polypeptide, or activating a PYR/PYL polypeptide or increasing expression of a PYR/PYL polypeptide, or a transcript encoding a PYR/PYL polypeptide.

1. PYR/PYL Polypeptide Binding Assays

Optionally, preliminary screens can be conducted by screening for agents capable of binding to a PYR/PRL polypeptide, as at least some of the agents so identified are likely PYR/PYL polypeptide modulators.

Binding assays can involve contacting a PYR/PYL polypeptide with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89). Other binding assays involve the use of mass spectrometry or NMR techniques to identify molecules bound to PYR/PYL polypeptide or displacement of labeled substrates (e.g., labeled ABA). The PYR/PYL polypeptide protein utilized in such assays can be naturally expressed, cloned or synthesized.

2. Activity

PYR/PYL polypeptide agonists can be identified by screening for agents that activate or increase activity of a PYR/PYL polypeptide. Antagonists can be identified by reducing activity.

One activity assay involves testing whether a candidate agonist can induce binding of a PYR/PYL protein to a type 2 protein phosphatase (PP2C) polypeptide in an agonist-specific fashion. Mammalian or yeast two-hybrid approaches (see, e.g., Bartel, P. L. et. al. Methods Enzymol, 254:241 (1995)) can be used to identify polypeptides or other molecules that interact or bind when expressed together in a cell. In some embodiments, agents that agonize a PYR/PYL polypeptide are identified in a two-hybrid assay between a PYR/PYL polypeptide and a type 2 protein phosphatase (PP2C) polypeptide (e.g., ABI1 or 2 or orthologs thereof, e.g., from the group A subfamily of PP2Cs), wherein an ABA agonist is identified as an agent that activates or enables binding of the PYR/PYL polypeptide and the PP2C polypeptide. Thus, the two polypeptides bind in the presence, but not in the absence of the agent. In some embodiments, a chemical compound or agent is identified as an agonist of a PYR/PYL protein if the yeast cell turns blue in the yeast two hybrid assay,

The biochemical function of PYR1, and PYR/PYL proteins in general, is to inhibit PP2C activity. This can be measured in live cells using the yeast two hybrid or other cell-based methods. It can also be measured in vitro using enzymatic phosphatase assays in the presence of a colorimetric detection reagent (for example, para-nitrophenylphosphate). The yeast-based assay used above provides an indirect indicator of ligand binding. To address this potential limitation, one can use in vitro competition assays, or cell based assays using other organisms, as alternate approaches for identifying weak binding target compounds.

3. Expression Assays

Screening for a compound that increases the expression of a PYR/PYL polypeptide is also provided. Screening methods generally involve conducting cell-based or plant-based assays in which test compounds are contacted with one or more cells expressing PYR/PYL polypeptide, and then detecting an increase in PYR/PYL expression (either transcript or translation product). Assays can be performed with cells that naturally express PYR/PYL or in cells recombinantly altered to express PYR/PYL, or in cells recombinantly altered to express a reporter gene under the control of the PYR/PYL promoter.

Various controls can be conducted to ensure that an observed activity is authentic including running parallel reactions with cells that lack the reporter construct or by not contacting a cell harboring the reporter construct with test compound.

4. Validation

Agents that are initially identified by any of the foregoing screening methods can be further tested to validate the apparent activity and/or determine other biological effects of the agent. In some cases, the identified agent is tested for the ability to effect plant stress (e.g., drought tolerance), seed germination, or another phenotype affected by ABA. A number of such assays and phenotypes are known in the art and can be employed according to the methods of the invention.

5. Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

The molecule of interest (e.g., PYR/PYL or a cell expressing a PYR/PYL polypeptide) can be bound to the solid state component, directly or indirectly, via covalent or non covalent linkage.

The invention provides in vitro assays for identifying, in a high throughput format, compounds that can modulate the expression or activity of PYR/PYL.

Abiotic stress resistance can assayed according to any of a number of well-known techniques. For example, for drought tolerance, plants can be grown under conditions in which less than optimum water is provided to the plant. Drought resistance can be determined by any of a number of standard measures including turgor pressure, growth, yield, and the like.

### VI. Methods of Increasing Abiotic Stress Tolerance in Plants

The present invention also provides methods of increasing abiotic stress tolerance in a plant. Thus, in some embodiments, a plant is contacted with an ABA agonist described herein, or an ABA agonist formulation, in sufficient amount to increase the abiotic stress tolerance in the plant. The amount of the ABA agonist formulation applied to the plant can be sufficient to increase the abiotic stress tolerance compared to not contacting the plant with the ABA agonist formulation. The plant can be contacted with the ABA formulation using any of the methods described herein. The increase in abiotic stress tolerance can improve the plants growth and/or survival to abiotic stress conditions that adversely effect the plant's growth or survival. Abiotic stress includes physical or chemical conditions described herein.

### VII. Methods of Inhibiting Seed Germination in a Plant

The present invention also provides methods of inhibiting seed germination. Thus, in some embodiments, a plant, plant part, or a seed is contacted with an ABA agonist formulation in an amount sufficient to inhibit seed germination. The seed can be contacted with the ABA formulation using any of the methods described herein. In some embodiments, the seed is directly contacted with the ABA agonist formulation. In some embodiments, the ground or soil is contacted with the ABA agonist formulation either prior to or after planting or sowing the seeds. In some embodiments, a plant is contacted with sufficient ABA agonist formulation to inhibit germination of seeds that later develop from the plant.

### VIII. Methods of Activating PYR/PYL Receptor Polypeptides

The present invention also provides methods of activating a PYR/PYL receptor polypeptide. In some embodiments, a PYR/PYL polypeptide is contacted with a compound described above, and the activated PYR/PYL polypeptide binds to a PP2C polypeptide. In some embodiments, the PYR/PYL polypeptide is capable of being activated by the agonist compound LC66C6. In some embodiments, the PYR/PYL protein that is activated is substantially identical to any one of SEQ ID NOs:1-119. Examples of sequences of ABA receptors from various plants are provided in U.S. Patent Publication 2011/0271408, which is incorporated by reference herein in its entirety.

In some embodiments, the method activates a PYR/PYL receptor in a cell free in vitro assay. In some embodiments, the method activates a PYR/PYL receptor expressed in a cell. In some embodiments, the cell also expresses a PP2C polypeptide. In some embodiments, the cell is a plant cell. In some embodiments, the cell is an animal or mammalian cell. In some embodiments, the cell expresses an endogenous PYR/PYL protein. In some embodiments, the cell is engineered to express a heterologous PYR/PYL polypeptide. In some embodiments, the cell expresses a heterologous PP2C polypeptide. In some embodiments, the cell expresses a PP2C polypeptide selected from HAB1 (homology to ABI1), ABI1, or ABI2.

In some embodiments, the activated PYR/PYL polypeptide induces expression of heterologous genes. In some embodiments, the heterologous genes are ABA responsive genes. In some embodiments, the induced gene expression occurs in cells that express an endogenous PYR/PYL polypeptide. In some embodiments, the induced gene expression occurs in cells that express a heterologous PYR/PYL polypeptide.

### EXAMPLES

### Example 1

This example demonstrates that novel ABA agonists described herein bind to and activate multiple PYR/PYL receptors.

### Methods

Chemical Screening

A previously described yeast two-hybrid system was used in high throughput screens (HTS) to identify ABA agonists (see, Peterson F C, et al. (2010) Structural basis for selective activation of ABA receptors. Nature Structural & Molecular Biology 17(9):1109-1111). In this system the agonist promoted receptor-PP2C interaction drives expression of a URA3 or HIS3 reporter gene and rescues uracil or histidine auxotrophy of parental strains (Peterson F C, et al. (2010); Vidal M, Brachmann R K, Fattaey A, Harlow E, & Boeke J D (1996) Reverse two-hybrid and one-hybrid systems to detect dissociation of protein-protein and DNA-protein interactions. Proceedings of the National Academy of Sciences of the United States of America 93(19):10315-10320). HTS were conducted using 5 different reporter strains that express binding domain (BD) fusions to PYR1, PYL1, PYL2, PYL3 or PYL4; these were co-expressed with activation domain (AD) fusions to HAB1 (pACT-HAB1); the constructs used have been described previously (Park et al. 2009). We utilized these strains in two separate screens. In the first screen 65,000 compounds obtained from Chembridge (San Diego, USA) were assayed for agonist activity using a halo assay, essentially as described by Gassner N C, et al. (2007) (Accelerating the discovery of biologically active small molecules using a high-throughput yeast halo assay. Journal of Natural Products 70(3):383-390). In this method yeast strains are embedded in selective agar and compounds pin transferred from 10 mM DMSO stock solutions onto assay plates; hits are evident by increased cell density in the vicinity of active compounds. Experiments using the halo assay utilized the yeast strain PJ69-4A and media supplemented with 10 mM 3-aminotriazole to improve selections. Halo screens were set up using a Biomek FX equipped with an automated microplate hotel (Thermo Cytomat) and a 384-pin tool (V & P Scientific), which was used to spot compounds on to assay plates. Prior to each chemical transfer the pins were washed in a 1:1 mixture of DMSO/water followed by a wash with 95% ethanol. After chemical transfer, plates were incubated at 28° C. and candidate agonists evident by manual inspection.

Although the halo screening method is powerful from the perspective of throughput, we subsequently employed a more conventional screening method for a second screen of a 12,000-member library obtained from Life Chemicals (Ukraine). This change was motivated by a desire to better control the assay concentration. In our second screen, reporter constructs were expressed in the yeast strain MAV99, which enables uracil-based selections via a GAL1 promoter driven URA3 transgene (Peterson F C, et al. (2010)). Screening compounds were added to selective uracil⁻ media seeded with reporter strains in 96 well plate format at a final concentration of 25 □M; yeast growth was inspected manually after ˜3 days. Compounds were transferred to screening wells from 2.5 mM stock solutions using a Biomek FX liquid handler.

As a third screening approach, the Life Chemicals library was also screened for Arabidopsis germination inhibitors in solidified agar medium containing 0.5×MS salts, 0.5% sucrose and 25 μM test compound. Hits from the germination assay were subsequently tested in yeast two hybrid assays. Hit compounds were restocked from their original vendors and used in secondary screens and compound characterization. Quinabactin and its analogs were purchased from Life Chemicals.

PP2C Activity Assay

HAB1 and PYL proteins were expressed and purified as described previously (Park S Y, et al. (2009) Abscisic Acid Inhibits Type 2C Protein Phosphatases via the PYR/PYL Family of START Proteins. Science 324(5930):1068-1071), with minor modifications. To obtain GST-HAB1, -ABI1 and -ABI2 fusion proteins, the HAB1 cDNA was cloned into pGex-2T whereas ABI1 and ABI2 cDNAs were cloned into the vector pGex-4T-1. Expression was conducted in BL21[DE3]pLysS host cells. Transformed cells were pre-cultured overnight, transferred to LB medium and cultured at 30° C. to culture A₆₀₀ of ˜0.5. The culture was then cooled on ice and MnCl₂ added to 4 mM and IPTG added to 0.3 mM. After 16 hours incubation at 15° C., cells were harvested and recombinant proteins were purified on glutathione agarose as described previously (Park S Y, et al. (2009). To obtain 6×His-PYL receptor fusion proteins, receptor cDNAs for all 13 ABA receptors were cloned into the vector pET28 and expressed and purified as described previously (Mosquna A, et al. (2011) Potent and selective activation of abscisic acid receptors in vivo by mutational stabilization of their agonist-bound conformation. PNAS 108(51):20838-20843); this yielded soluble and functional protein (assessed using receptor-mediated PP2C inhibition assays) for all receptors except PYL7, PYL11 and PYL12. These three receptors were therefore alternatively expressed as maltose binding (MBP) fusion proteins using the vector pMAL-c; expression of these constructs was carried out in BL21 [DE3]pLysS host strain with the same induction conditions used for GST-HAB1. Recombinant MBP-PYL fusion proteins were purified from sonicated and cleared lysate using amylose resin (New England Biolab, Inc.) using the manufacturers purification instructions. This effort yielded an active MBP-PYL11 fusion protein, but failed for PYL7 and PYL12.

PP2C activity assays using recombinant receptors and PP2Cs were carried out as follows: Purified proteins were pre-incubated in 80 μl assay buffer containing 10 mM MnCl₂, 3 μg bovine serum albumin and 0.1% 2-mercaptoethanol with ABA or ABA agonist for 30 minutes at 22° C. Reactions were started by adding 20 μL of a reaction solution containing 156 mM Tris-OAc, pH 7.9, 330 mM KOAc and 5 mM 4-methylumbelliferyl phosphate after which fluorescence measurements were immediately collected using an excitation filter 355 nm and an emission filter 460 nm on a Wallac plate reader. Reactions contained 50 nM PP2C and 100 nM PYR/PYL proteins, respectively.

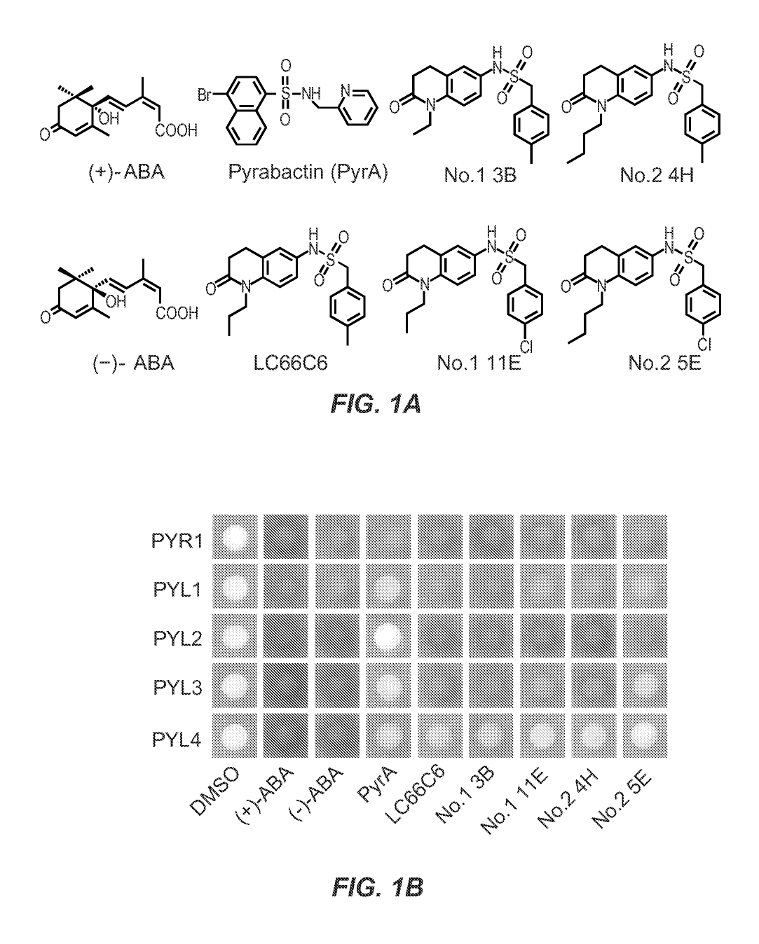

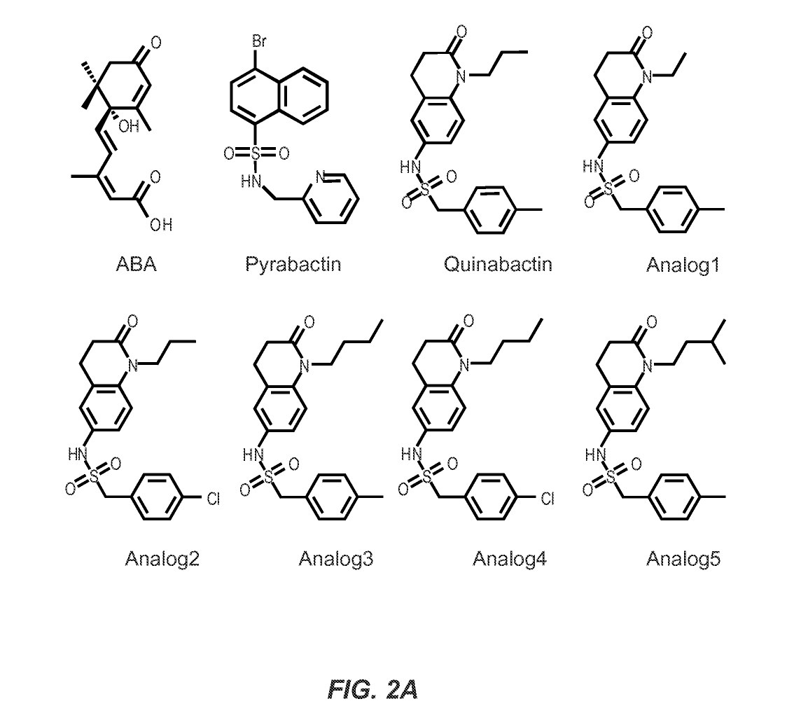

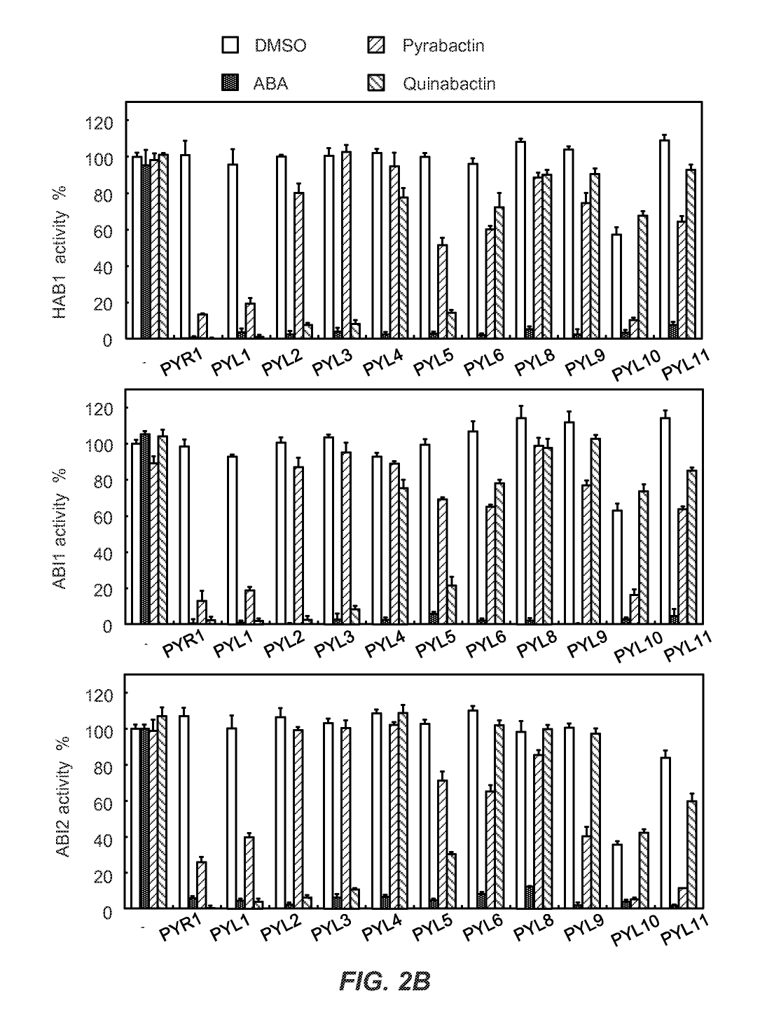

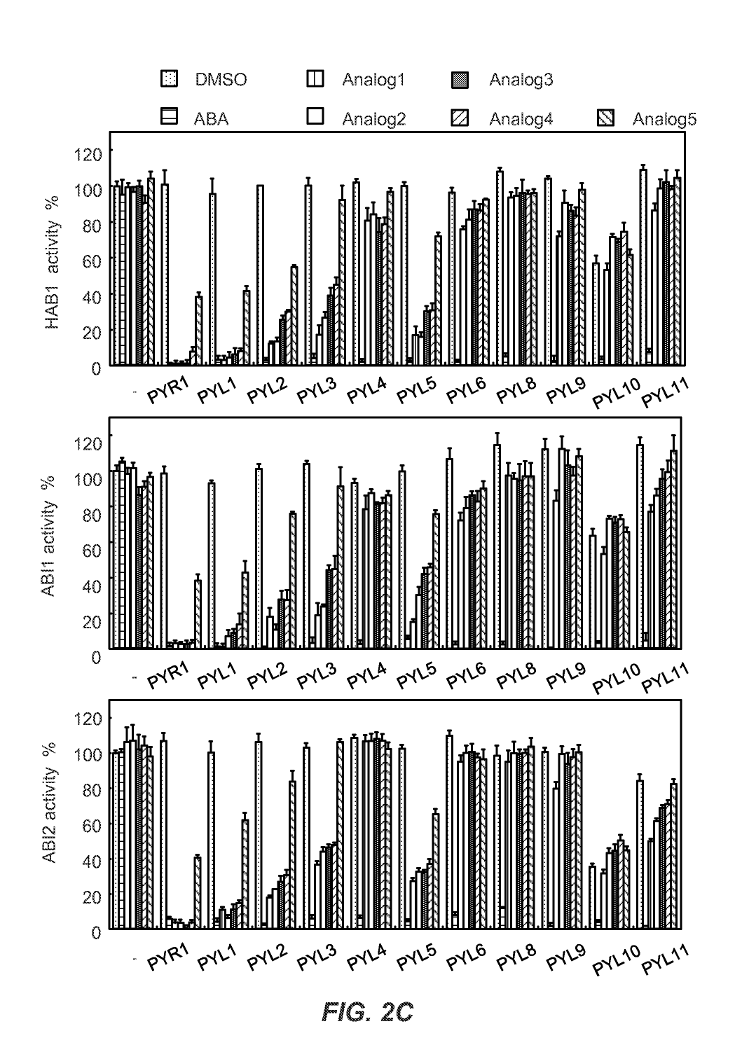

FIG. 1A shows a representative group of ABA agonists. As shown in FIG. 1B, multiple PYR/PYL receptors are activated by several agonists, including LC66C6, in a yeast two-hybrid assay. This assay reports the agonist-promoted physical interaction of PYR/PYL proteins and Glade A PP2C proteins when a specific receptor and PP2C are fused to GAL4 activation and DNA-Binding domains respectively, as previously described (Park et al. 2009). These yeast-based assays indicate that LC66C6 is an agonist of multiple PYR/PYL receptors, unlike the previously described agonist pyrabactin, which has much greater receptor selectivity than ABA or the new agonist LC66C6. As previously described, the agonist-promoted binding of a receptor to a Glade A PP2C inhibits the PP2C's phosphatase activity. In Arabidopsis, there are 14 PYR/PYL receptors, 13 of which can mediate ABA-responses in a protoplast-based assay system (Fujii et al. 2009). To examine LC66C6's selectivity more closely, we attempted to express and purify recombinant 6×-His-PYR/PYL proteins for all 14 members and recovered ABA-responsive receptors for all receptors except PYL7, 12 and 13, which could not be produced in active forms for technical reasons. This panel of recombinant receptors enables a near complete portrait of an ABA-agonists activity on members of the Arabidopsis PYR/PYL receptor family. As shown in FIG. 2, the PPC2 enzyme activity of HBA1, ABI1, and ABI2 is inhibited by >90% by 10 μM ABA in the presence of all ABA receptors tested (FIG. 2B). In response to LC66C6 (Quinabactin), >70% PP2C inhibition of HBA1, ABI1, and ABI2 was observed with the receptors PYR1, PYL1, PYL2, PYL3 and PYL5.

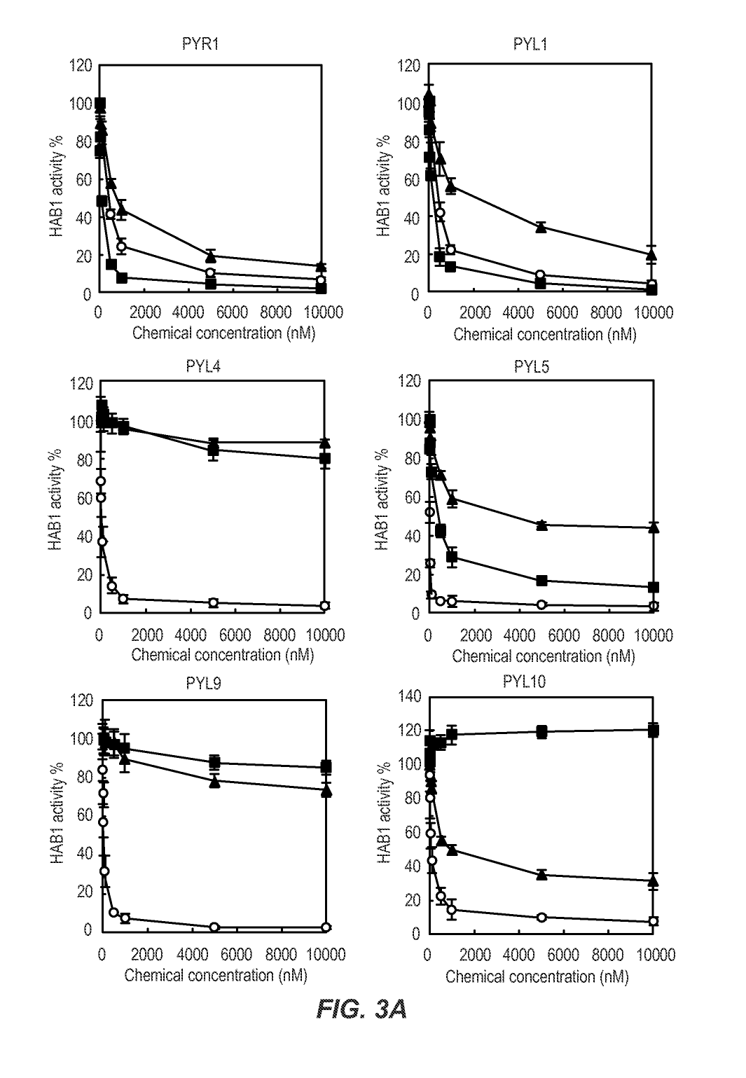

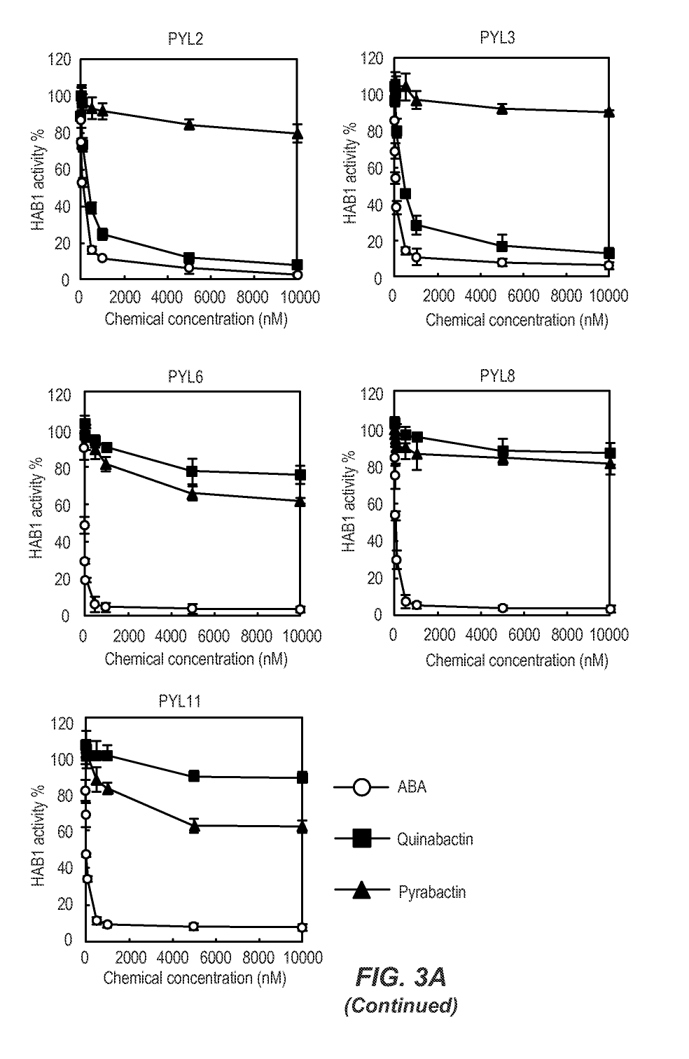

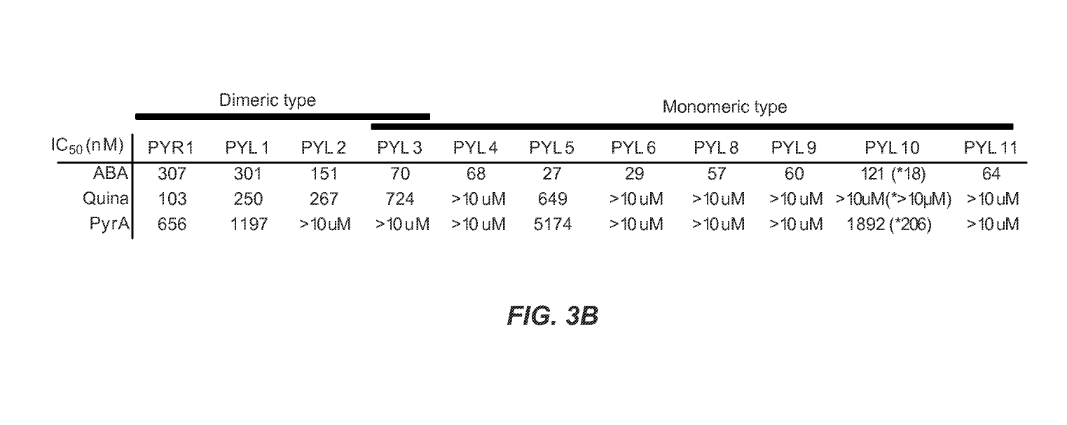

To further characterize quinabactin's activity and define its receptor selectivity, receptor-mediated PP2C-inhibition assays were conducted using 10 recombinant receptors in combination with the PP2Cs HAB1, ABI1 or ABI2. These experiments showed that quinabactin activates PYR1, PYLs 1-3 and PYL5 with submicromolar IC₅₀ values and displays substantially higher activity at dimeric receptor sites (FIGS. 2, 3 and 4). The results also show that quinabactin is a stronger PYR1 or PYL1 agonist than ABA (FIGS. 2 and 3). In addition, the maximal PP2C inhibition observed by quinabactin was higher than that observed with pyrabactin with all receptors tested. Although pyrabactin can activate PYL5 with an IC50 of 0.90 μM, it saturates at ˜40% PP2C inhibition, suggesting that it is an incomplete/partial PYL5 agonist. Thus, this example demonstrates the identification of a new sulfonamide agonist with broader receptor spectrum activity and increased bioactivity relative to pyrabactin.

### Example 2

This example demonstrates that novel ABA agonists inhibit germination and plant growth.

Arabidopsis Germination and Hypocotyl Growth Inhibition Analysis

For Arabidopsis germination and hypocotyl growth inhibition analysis, seeds after-ripened about 4 weeks were surface-sterilized with a solution containing 5% NaClO and 0.05% Tween-20 for 10 minutes, and rinsed with water four times. Sterilized seeds were suspended with 0.1% agar and sowed on the 0.8% solidified agar medium containing ½ Murashige and Skoog (MS) salts (Sigma-Aldrich) in the presence of chemicals and were stored at 4° C. for 4 days, then transferred at 22° C. under the dark or light. Germination was determined after a 4-day incubation, whereas hypocotyl growth was photographed after 6-day incubation.

Plant Materials

The following alleles/mutant strains were used: aba2-1 (Leon-Kloosterziel K M, et al. (1996) Isolation and characterization of abscisic acid-deficient Arabidopsis mutants at two new loci. Plant J 10(4):655-661), abi1-1 (Umezawa T, et al. (2009) Type 2C protein phosphatases directly regulate abscisic acid-activated protein kinases in Arabidopsis. Proceedings of the National Academy of Sciences of the United States of America 106(41):17588-17593), abi3-9, abi4-11 (Nambara E, et al. (2002) A screen for genes that function in abscisic acid signaling in Arabidopsis thaliana. Genetics 161(3):1247-1255), and pry1pyl1pyl2ply4 quadruple (Park S Y, et al. (2009) Abscisic Acid Inhibits Type 2C Protein Phosphatases via the PYR/PYL Family of START Proteins. Science 324(5930):1068-1071); all of these strains are in the Columbia background. The pry1pyl1pyl2ply4 quadruple mutant stain utilized was backcrossed to Columbia three times. Barley and soybean seeds were purchased from Living Whole Foods, Inc., whereas maize seeds were obtained W. Atlee Burpee & Co. Detail methods used for physiological experiments using these materials are provided as supporting information.

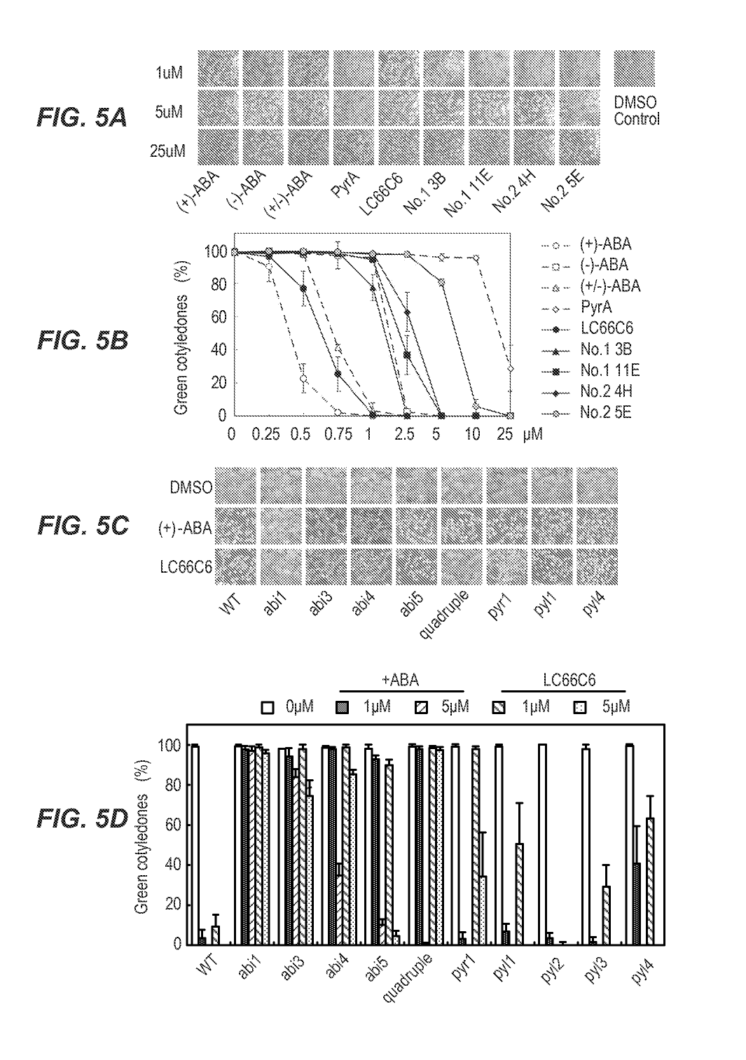

To explore the physiological consequences of LC66C's unique agonist properties, we characterized its effects on Arabidopsis seeds, seedlings and adult plants. As shown in FIG. 5, the ABA agonists described herein strongly inhibit seed germination in Arabidopsis. FIGS. 5A and 5B show that several agonists, including LC66C6, inhibit germination of seeds in a dose dependent manner. In particular, LC66C6 was nearly as effective, on a per mole basis, at inhibiting germination as (+)-ABA, and was more effective than the other agonists tested.

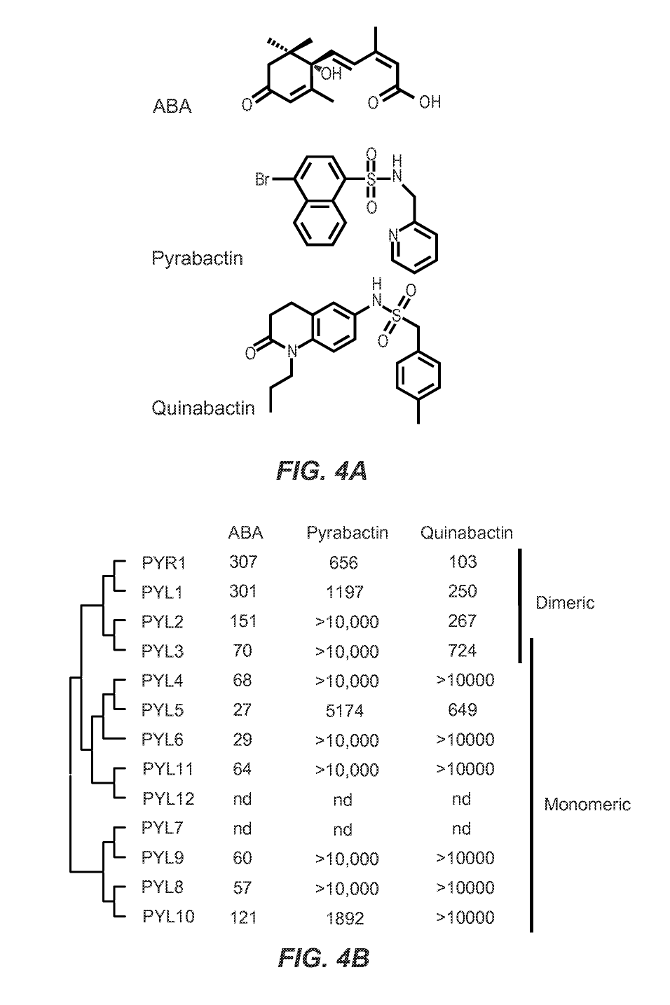

FIGS. 5C and 5D show the effect of agonists (+)-ABA and LC66C6 on inhibiting germination of seeds from various ABA-insensitive mutants. As shown in FIG. 5C, at a concentration of 5 μM, LC66C6 showed a similar pattern of inhibiting germination as (+)-ABA did for all mutants tested except for the PYR/PYL quadruple mutant (pyr1/pyl1/pyl2/pyl4) and pyr1 single mutant. Combined with the IC₅₀ data presented above in FIG. 4, this genetic data suggests that the germination-inhibitory activity of LC66C6 is largely explained by its ability to agonize PYR1, PYL1 and PYL2. The ability of ABA to inhibit germination in the quadruple mutant is likely explained by its agonist activity on other receptors. Our genetic data are consistent with the hypothesis that PYR1 plays an important but redundant role in seed germination in response to ABA, as the pyr1 mutant germinates in the presence of either 5 μM LC66C6 or pyrabactin (Park et al. 2009).

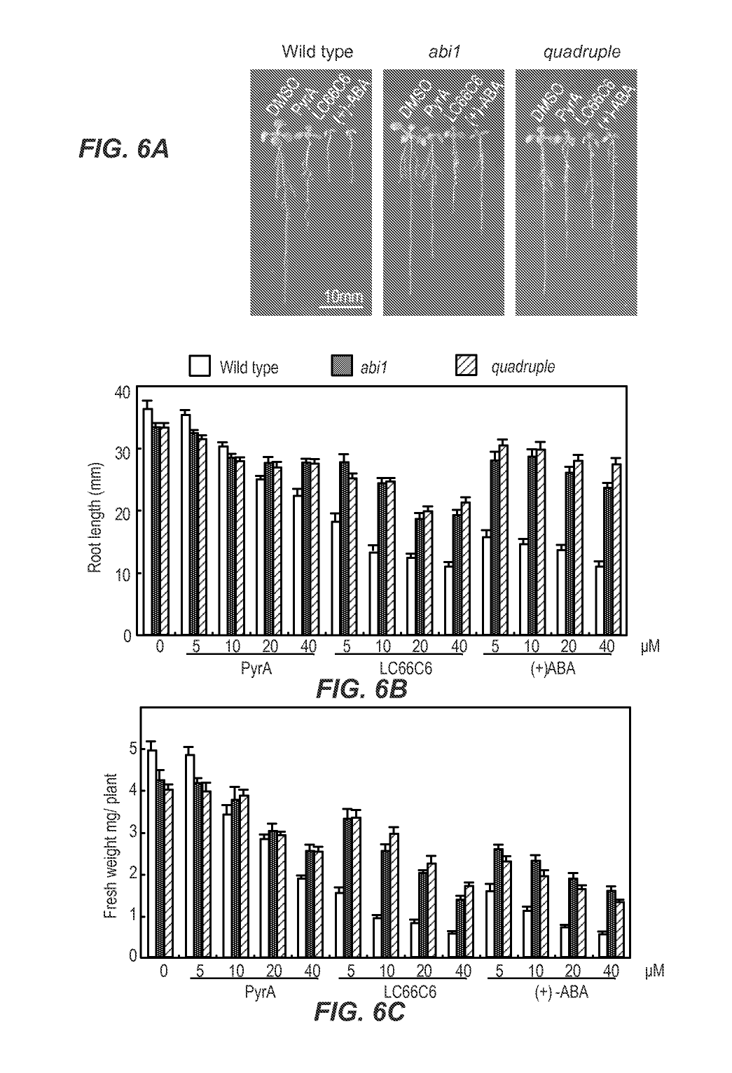

As shown in FIG. 6, LC66C6 also inhibits plant growth after germination. FIGS. 6A and 6B show that LC66C6 inhibits root elongation in wild-type, abi1, and the quadruple mutant, and is comparable to or slightly more effective than (+)-ABA in its inhibitory effects at all concentrations tested. Further, FIG. 6C demonstrates that LC66C6 inhibits growth of both wild-type and mutant plants in a concentration dependent manner. The inhibition of plant growth by LC66C6 is significantly greater than the inhibition by pyrabactin, and comparable to that of (+)-ABA.

This example demonstrates that LC66C6 is a potent inhibitor of seed germination and growth of both wild-type and ABA-insensitive mutant plants.

### Example 3

This example demonstrates that agonist LC66C6 induces drought stress tolerance.

Physiological Assays

Physiological assays were performed on Arabidopsis plants grown at 22±2° C. and relative humidity (RH) 45±10% under a 16/8-h light/dark cycle. For transpirational water loss analyses in Arabidopsis, plants were pre-treated by aerosol spray of 4 ml solution containing 25 μM compound and 0.05% Tween-20. 12 4-week old plants were sprayed per compound or control analyzed. After overnight pre-treatment with compounds, the aerial portions were detached from roots, and their fresh weight measured at 20 min intervals over a 2 hour time period. To measure stomatal aperture, plants were pre-treated with compounds as described above, covered with plastic lids to maintain high RH and after overnight pre-treatment leaf epidermal impressions were obtained using Suzuki's Universal Micro-Printing (SUMP) method using SUMP impression solution with SUMP B plates (SUMP Laboratory). The leaf impressions were analyzed by light microscopy and stomatal apertures were determined from the pore widths using ImageJ 1.43v software (National Institutes of Health, USA). For Arabidopsis drought stress assays, approximately 1.5 ml of a 25 μM chemical solution was applied by aerosol to plants at daily intervals over a 3 day period. Plants were grown in square 6×6×5 cm pots containing 100 g soil per pot. Soybean drought stress assays were performed on plants grown at 25±2° C., 65±10% RH under a 16/8-h light/dark cycles. Approximately 20 ml of a 50 μM chemical solution containing 0.05% Tween-20 was sprayed per pot (3 plants per pot) four times each 3 days. Pots used were 250 ml size, and contained 200 g soil per pot. Pots were covered in Parafilm to so that the water loss measured was transpiration mediated. Soil water content % was determined by measuring pot weight and computed by removing dry soil weight from total weight.

Water loss analyses in soybean, barley and maize.

For water loss analyses using soybean barley and maize, 100 μM chemical solution containing 0.05% Tween-20 was sprayed on to the aerial parts of the plants. The soybean, barley and maize plants used were approximately 4-, 2- and 2-weeks old respectively. Compounds were applied 16 hours before water loss assays were conduction. To measure water loss entire shoots were detached and their fresh weight monitored.

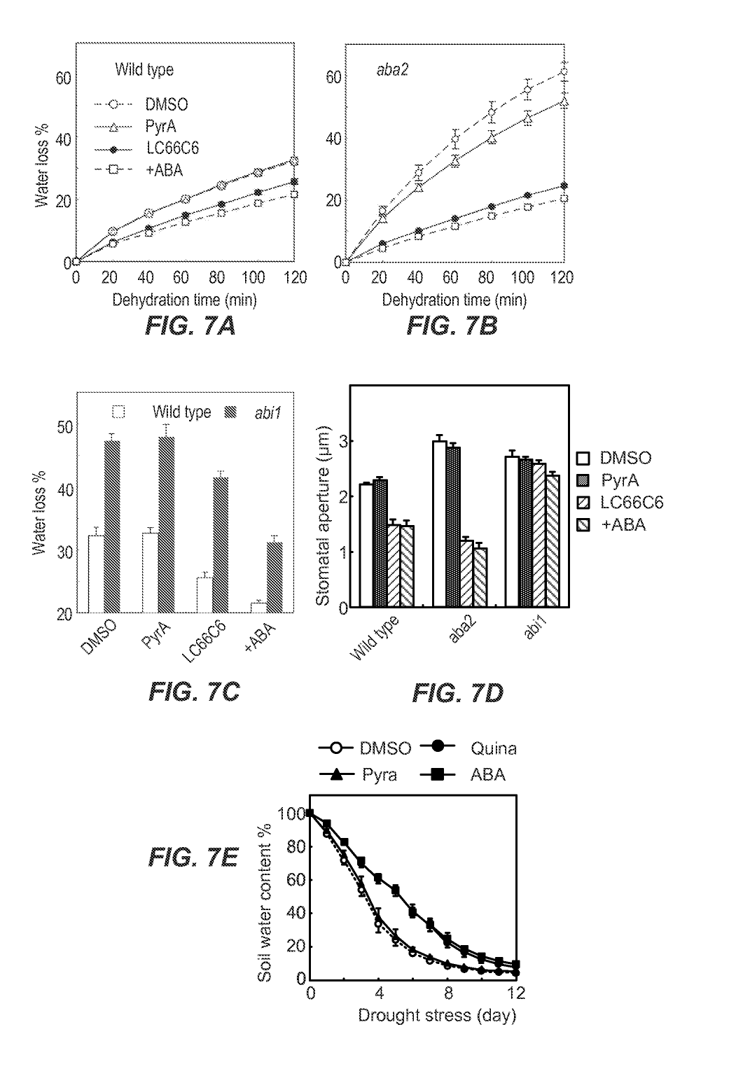

FIG. 7 shows the effect of LC66C6 on various parameters related to drought stress. As shown in FIGS. 7A and 7B, LC66C6 reduced the amount of transpirational water loss in detached leaves from wild-type and aba2 (ABA-deficient mutant 2) mutant plants. However, as shown in FIG. 7C, LC66C6 did not reduce transpirational water loss in detached leaves from the abi1-1 mutant. FIG. 7D shows that LC66C6 induces stomatal closure in wild-type and the aba2 mutant, but not in the abi1-1 mutant. FIG. 7E shows the effects of agonist compounds on soil water content during drought treatment of soybean plants.

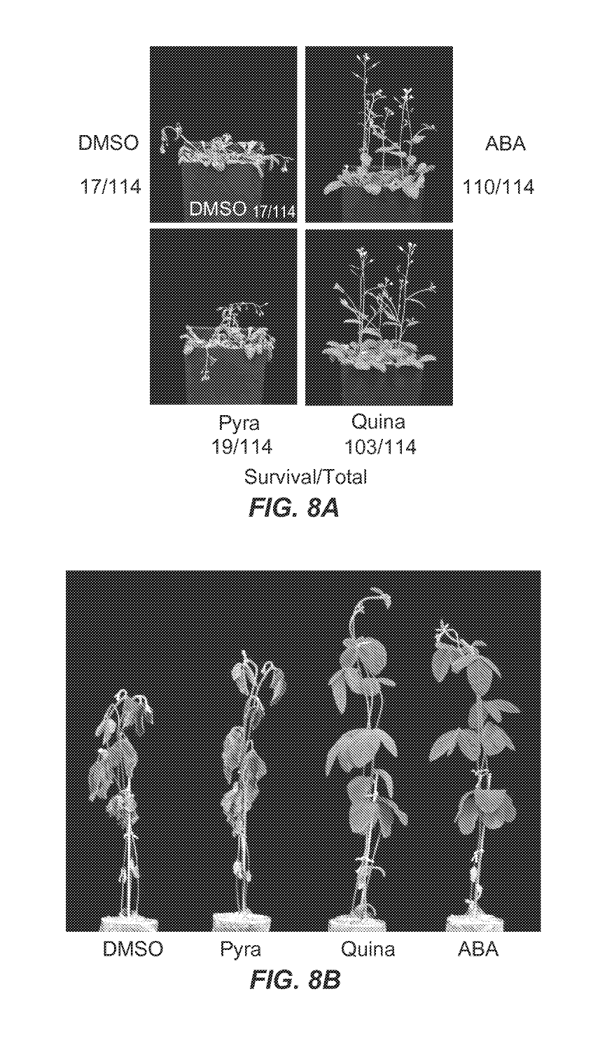

FIG. 8A shows that treatment of plants with quinabactin confers drought stress tolerance in Arabidopsis plants similar to that conferred by treatment with (+)-ABA. In this example, two-week-old plants were subjected to drought stress by withholding water and were photographed after 12 days. Plants were re-hydrated after 2 weeks drought treatment. The number of surviving plants per total number of tested plants is shown adjacent to the photographs. FIG. 8B shows that treatment of soybean plants with quinabactin confers drought stress tolerance similar to that conferred by treatment with (+)-ABA. In this example, two-week-old plants were subjected to drought stress by withholding water and photographed after 8 days of drought treatment. For all drought stress treatments, compounds (tested at 25 μM for Arabidopsis and 50 μM for soybean) were applied in solutions containing 0.05% Tween-20 and applied as aerosols every 3 days over the drought regime. Values for all experiments are means±SEM (n=6, 3 plants used per experiment).

This example shows that LC66C6 induces drought stress tolerance in wild-type and aba2 mutant Arabidopsis plants and in wild-type soybean plants similar to that conferred by (+)-ABA.

### Example 4

This example demonstrates the LC66C6 induces ABA-responsive genes in a manner similar to those induced by (+)-ABA.

Microarray Analyses

Total RNA was isolated using RNAeasy Plant Mini Kit (Qiagen, USA) according to the manufacturer's instructions. cDNA synthesis, labeling and hybridization to the Arabidopsis ATH1 chips (Affymetrix, USA) were performed by the IIGB Core Instrumentation Facility of University of California at Riverside using Affymetrix protocols. Biological triplicate samples were hybridized for DMSO controls, ABA, pyrabactin and quinabactin treatments; compound were applied at 25 μM final concentration and RNA prepared from frozen tissue after 6 hours exposure to compounds or controls. Expression signals for probe sets were calculated and normalized by MASS Statistical Algorithm (Affymetrix, USA). Experimental filtering of array data was performed for the presence of signal in all experiments. Average transcript levels in each chemical treatment were compared to those in control experiments and used to compute to fold-change values. Log₂-transformed fold-change values were used to compute Person Correlation Coefficients between experimental conditions.

Quantitative RT-PCR Analysis

Total RNA was isolated using Plant RNA purification reagent (Invitrogen, USA) according to the manufacturer's instructions. cDNA was synthesized from 1 μg of total RNA using the QantiTec reverse transcription kit (Qiagen, USA). Real-time PCR using Maxima® SYBR Green/Fluorescein qPCR Master Mix (Fermentas) was performed with the iQ5 real-time PCR detection system (Bio-Rad, Hercules, Calif.). The relative amounts of target mRNAs were determined using the relative standard curve method and were normalized by relative amount of internal control mRNA. Biological triplicate experiments were performed. The primer sequences used in these experiments are shown in Table 2.

TABLE 2
Primer sets for quantitative RT-PCR

| AGI gene code | Abbreviation | Forward primer | Reverse primer |
|---|---|---|---|
| Arabidopsis | | | |
| AT1G05100 | MAPKKK18 | AAGCGGCGCGTGGAGAGAGA | GCTGTCCATCTCTCCGTCGC |
| | | (SEQ ID NO: 120) | (SEQ ID NO: 121) |
| AT5G52310 | RD29A | TGAAGTGATCGATGCACCAGG | GACACGACAGGAAACACCTTTG |
| | | (SEQ ID NO: 122) | (SEQ ID NO: 123) |
| AT5G52300 | RD29B | TATGAATCCTCTGCCGTGAGAGGT | ACACCACTGAGATAATCCGATCCT |
| | | G (SEQ ID NO: 124) | (SEQ ID NO: 125) |
| AT4G34000 | ABF3F | GTTGATGGTGTGAGTGAGCAGC | AACCCATTACTAGCTGTCCCAAG |
| | | (SEQ ID NO: 126) | (SEQ ID NO: 127) |
| AT2G46270 | GBF3 | GACGCTTTTGAGCATCGACACT | ACTGTTTCCTTCGCTCCCGTTTC |
| | | (SEQ ID NO: 128) | (SEQ ID NO: 129) |
| Internal control | ACT2 | CTCATGAAGATCCTTACAG | CTTTCAGGTGGTGCAACGAC |
| | | (SEQ ID NO: 130 | (SEQ ID NO: 131) |
| Soybean | GmNAC4 | ACGTCAGTTCCGCAAAAGAT | GGACCCGTTGGTTTCTCAC |
| | | (SEQ ID NO: 132) | (SEQ ID NO: 133) |
| | GmbZIP1 | GGGAATGGGAATTTGGGTGAGAA | CCTTCTGCCAGGGCTAGCATG |
| | | (SEQ ID NO: 134) | (SEQ ID NO: 135) |
| Internal control | Gm18S | CCTGCGGCTTAATTTGACTCAAC | TAAGAACGGCCATGCACCA |
| | | (SEQ ID NO: 136) | (SEQ ID NO: 137) |
| Barley | HVA1 | AACACGCTGGGCATGGGAG | CGAACGACCAAACACGACTAAA |
| | | (SEQ ID NO: 138) | (SEQ ID NO: 139) |
| | HvDRFI | CGGGCGGCGCGATTGCGAGC | ACGGAATTAGGGCCATCACG |
| | | (SEQ ID NO: 140) | (SEQ ID NO: 141) |
| Internal control | Hvtubulin2 | TCCATGATGGCCAAGTGTGA | GACATCCCCACGGTACATGAG |
| | | (SEQ ID NO: 142) | (SEQ ID NO: 143) |
| Maize | ZmLEA | GCAGCAGGCAGGGGAGAA | GCCGAGCGAGTTCATCATC |
| | | (SEQ ID NO: 144) | (SEQ ID NO: 145) |
| | ZmRAB17 | ATGAGTACGGTCAGCAGGGGCAG | CTCCCTCGCAGGCTGGAACTG |
| | | (SEQ ID NO: 146) | (SEQ ID NO: 147) |
| Internal control | ZmUbi | TGCCGATGTGCCTGCGTCGTCTGG | TGAAAGACAGAACATAATGAGCACAG |
| | | TGC (SEQ ID NO: 148) | (SEQ ID NO: 149) |

ABA-Responsive Reporter Gene Assays

Existing ABA-responsive promoter-GUS fusions are, in our experience, not ideal due to either high background levels or relatively low induction levels in response to ABA. MAPKKK18 as a highly-ABA inducible gene with low background levels (Matsui A, et al., Plant Cell Physiol 49(8):1135-1149 (2008)); MAPKKK18 is also strongly induced by drought and salt stress. We therefore characterized the effects of agonists on MAPKKK18 promoter::GUS reporter transgenic plants. GUS staining was performed in a reaction buffer of the following composition: 50 mM sodium phosphate buffer pH 7.0, 0.05% Tween-20, 2.5 mM potassium ferrocyanide, 2.5 mM potassium ferricyanide, 1 mM X-gluc. The reaction buffer was vacuum infiltrated into test samples for 10 min two times and then incubated at 37° C. for 5 h. The reaction was stopped by washing the samples with 70% ethanol, and chlorophyll pigments bleached by incubation at 65° C.

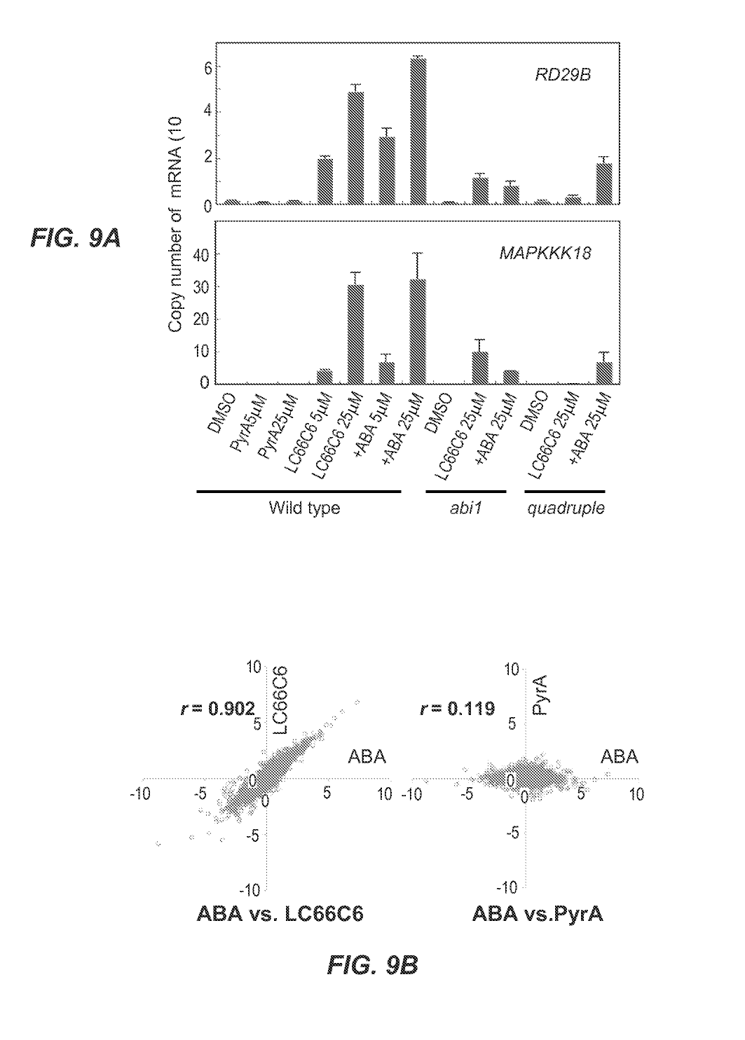

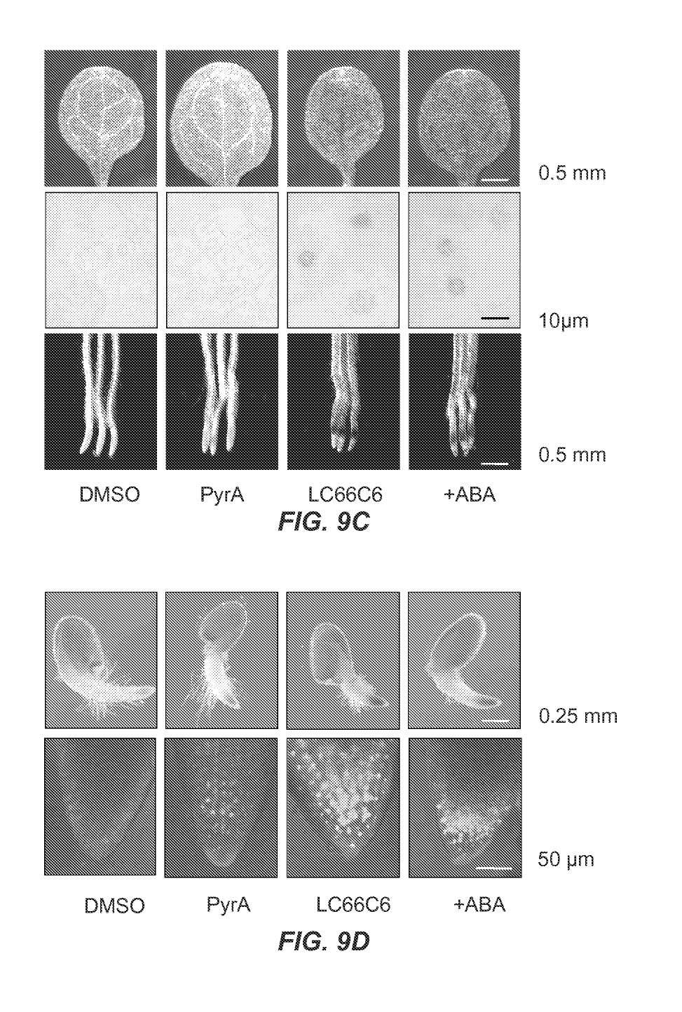

FIG. 9 shows gene expression changes induced in response to pyrabactin, LC66C6, and (+)-ABA. As shown in FIG. 9A, LC66C6 induced the expression of RD29B and MAPKKK18 mRNA in a dose dependent manner in wild-type plants, whereas those induction levels impaired in both abi1-1 and PYR/PYL quadruple mutant plants. The induction of gene expression by LC66C6 is similar to that observed with (+)-ABA. In contrast to (+)-ABA and LC66C6, pyrabactin did not induce gene expression in wild-type plants, although it does induce modest ABA-related gene expression in seedings when higher concentrations are utilized in treatment (Park et al., 2009).

FIG. 9B shows genome-wide comparison of ABA and LC66C or pyrabactin effects, in comparison to control treatments, on the wild-type seedlings, as measured by hybridization of labeled RNAs to ATH1 microarrays. As shown in FIG. 9B, LC66C6 induces a similar set of genes to those induced by ABA in a microarray experiment. In contrast, pyrabactin did not induce an expression pattern similar to that of ABA.

FIGS. 9C and 9D show that LC66C6 induces expression of reporter genes in the same tissues as (+)-ABA. The expression of reporter genes was observed in guard cells and vascular tissues of leaves and roots, and in radicle tips of imbibed seeds.

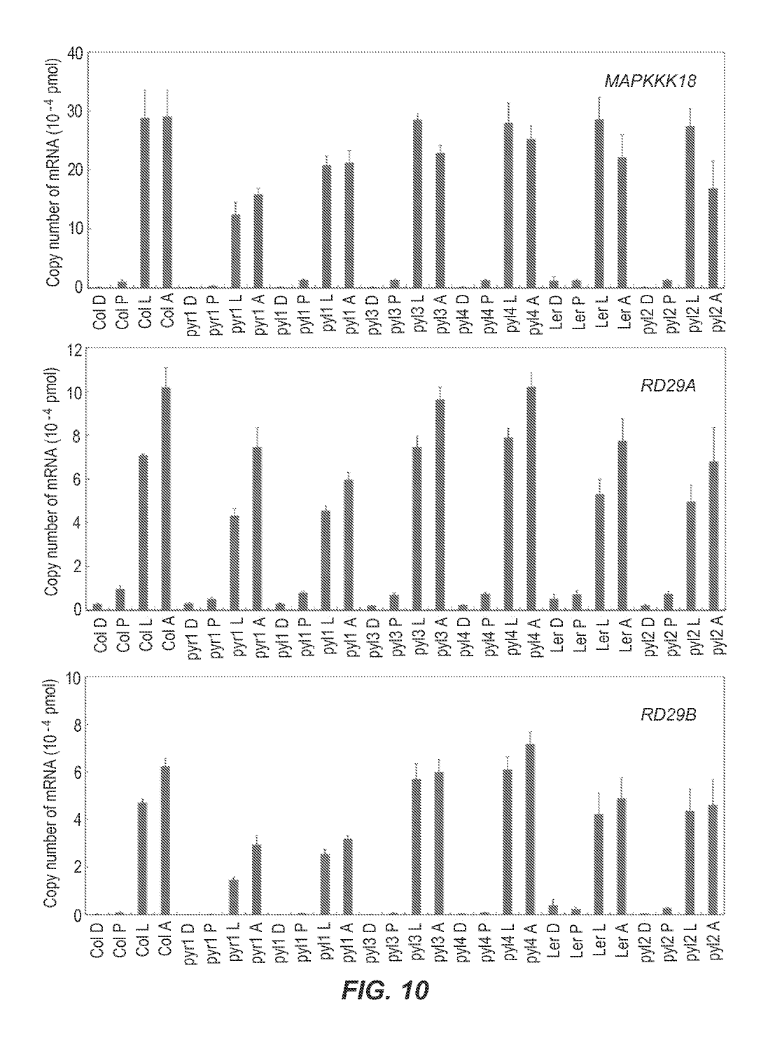

FIG. 10 shows ABA-responsive gene expression in PYR/PYL single mutants. As shown in FIG. 10, the ABA-responsive MAPKKK18, RD29A, and RD29B mRNAs were induced by both LC66C6 and (+)-ABA in the Col and Ler ecotypes and the pyr1, pyl1, ply2, pyl3 and pyl4 single mutant genotypes. In contrast, pyrabactin did not significantly induce expression of any of the genes assayed in any of the single mutants or wild-type ecotypes.

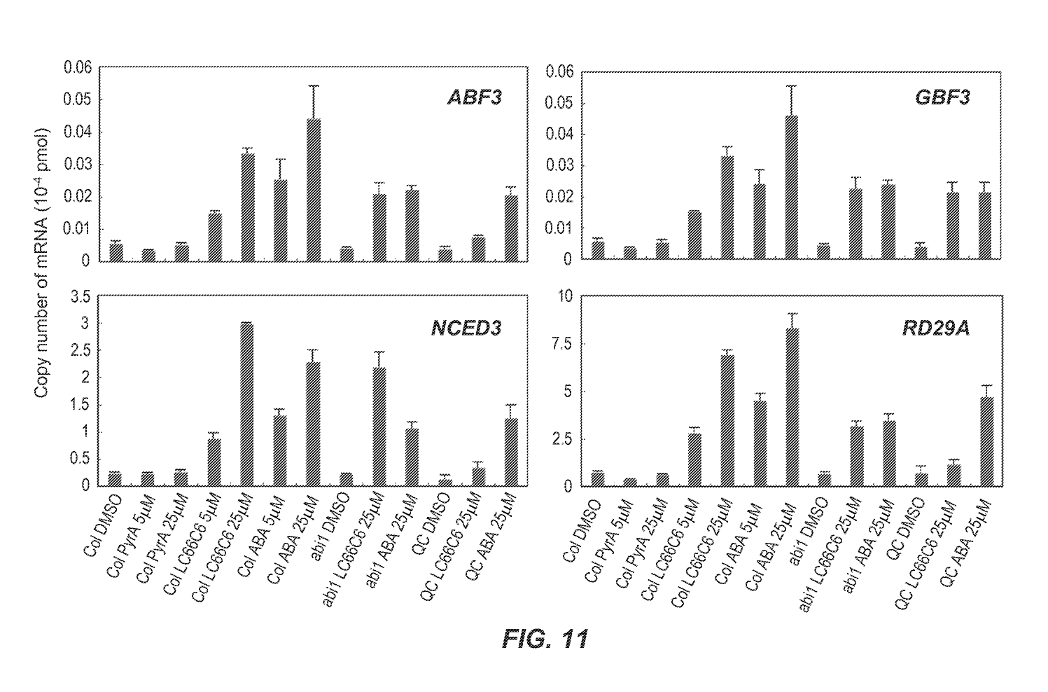

FIG. 11 shows ABA-responsive gene expression in wild-type plants, abi1-1 and PYR/PYL quadruple mutants. As shown in FIG. 11, both LC66C6 and (+)-ABA induced expression of ABF3, GBF3, NCED3, and RD29A in a dose dependent manner in Col wild-type plants, whereas the induction levels were impaired in both abi1-1 and PYR/PYL quadruple mutant plants. Consistent with the above results, pyrabactin did not induce significant expression of any genes analyzed in the wild-type plants.

### Example 5

This example demonstrates that key enzymes for ABA catabolism do not affect the responses induced by LC66C6.

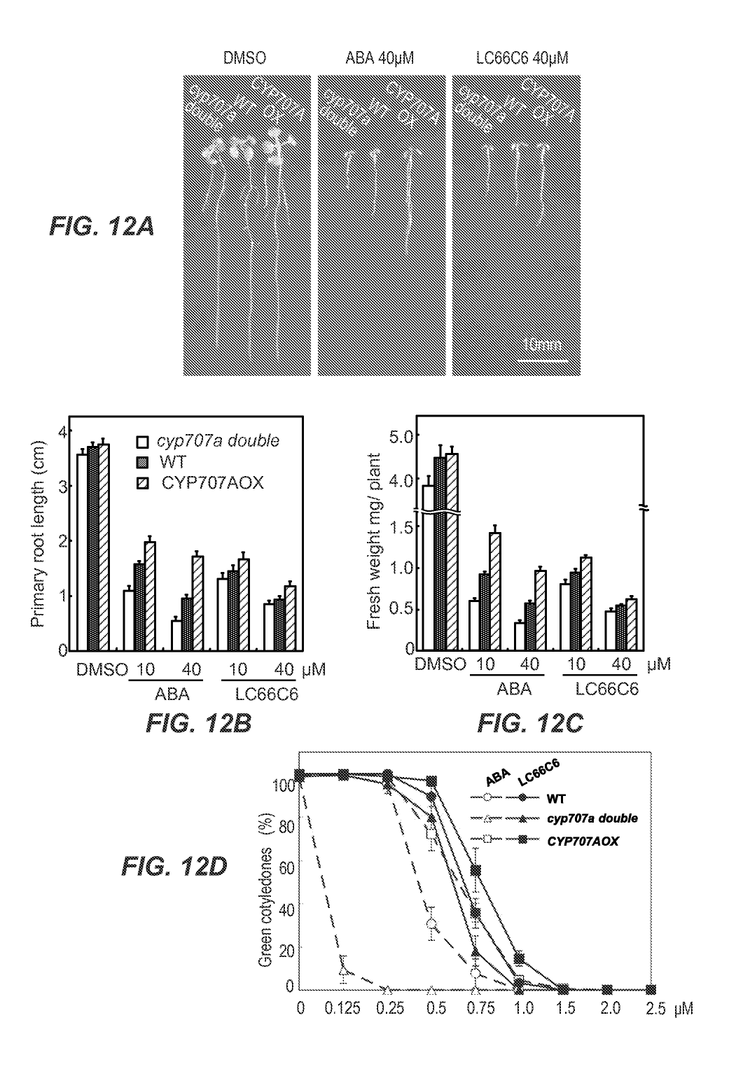

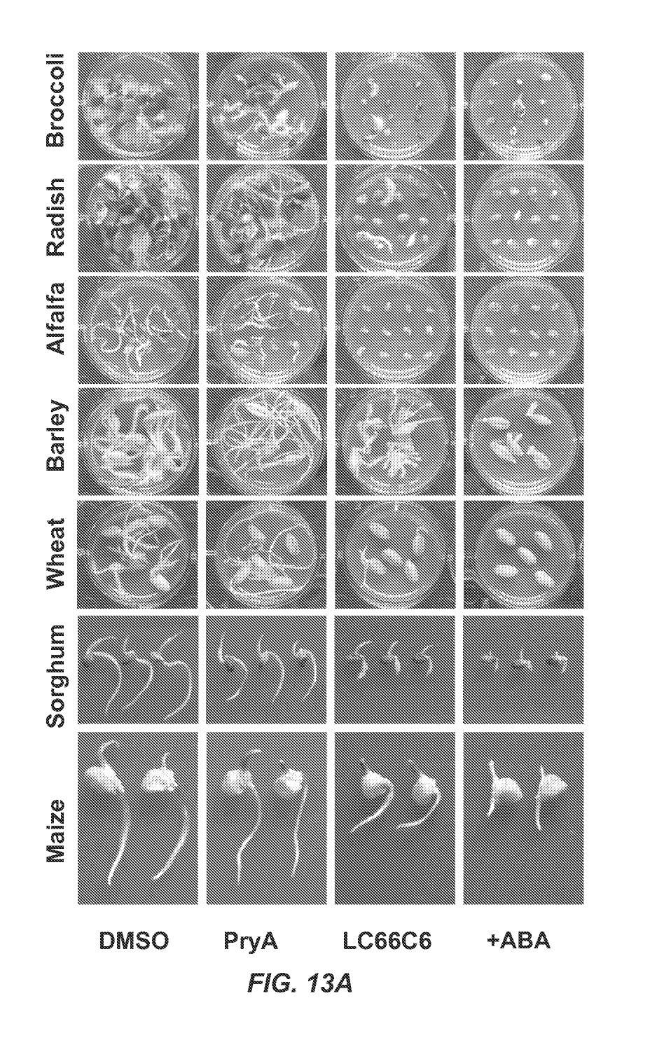

As shown in FIG. 12, the inhibition of plant growth and germination by ABA is enhanced in plants that are double mutant for cyp707a, a key enzyme for ABA catabolism, but is reduced in plants that overexpress CYP707A (CYP707AOX; see FIGS. 12A-D). In contrast, the effects on plant growth and germination by LC66C6 are not significantly different in plants that are double mutant for cyp707a, wild-type plants, or in plants that overexpress CYP707AOX (see FIGS. 12A-D).

This example shows that enzymes that are involved in the breakdown of ABA do not influence the phenotypes regulated by LC66C6.

### Example 6

This example shows that LC66C6 is bioactive on diverse plant species, including monocots and dicots.

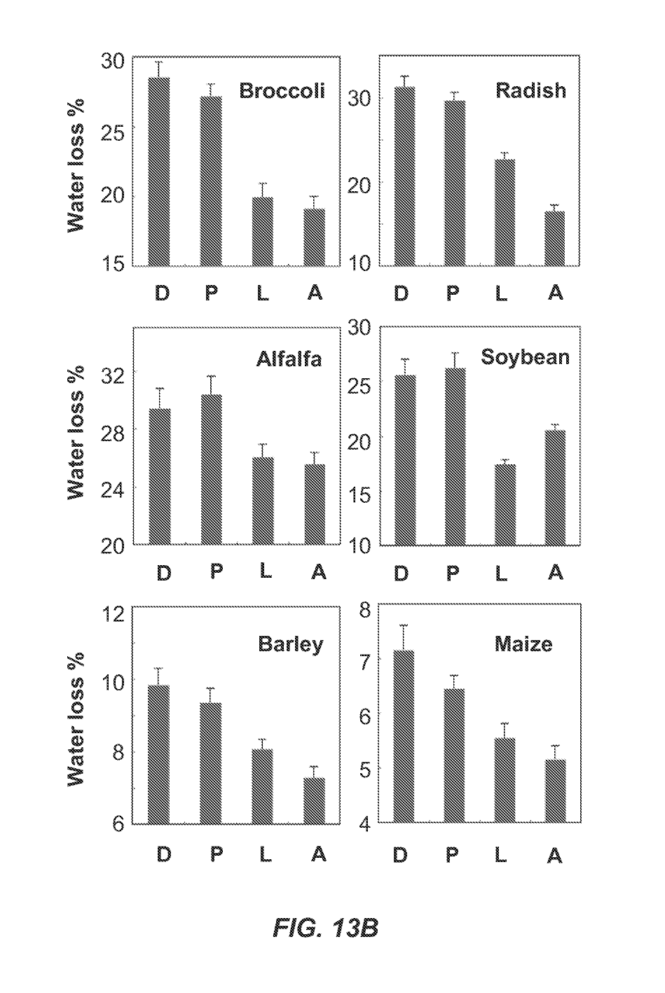

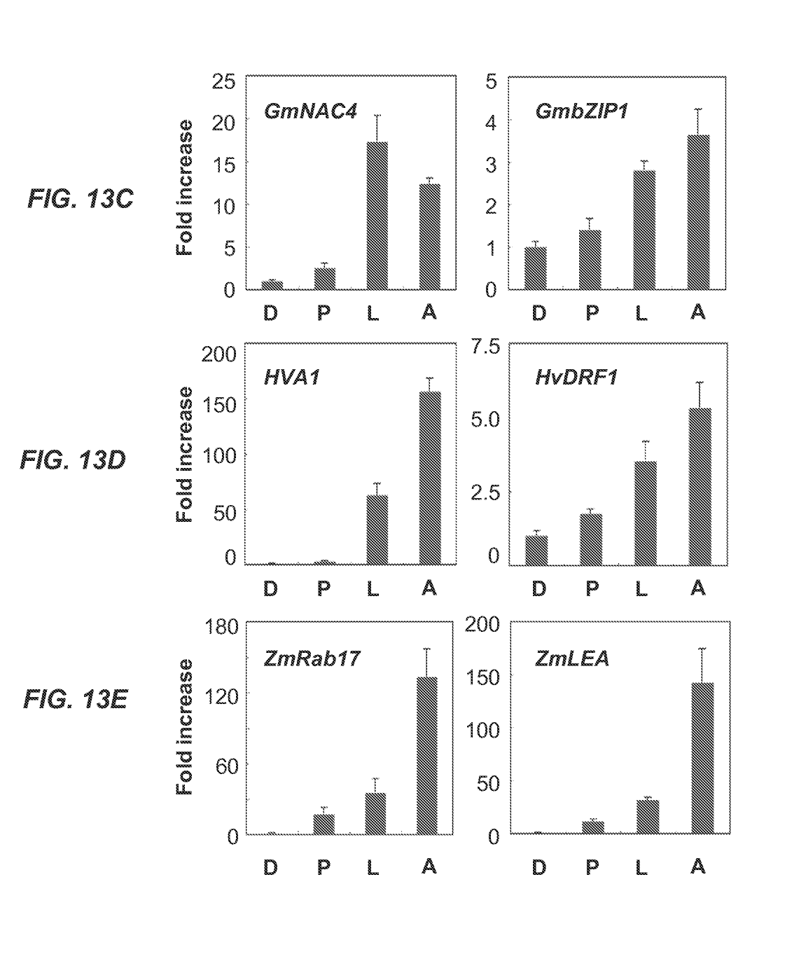

FIG. 13A shows that LC66C6 inhibits germination of broccoli, radish, alfalfa, soybean, barely, wheat, sorghum and maize seeds. The level of inhibition of germination by LC66C6 is greater than pyrabactin. As shown in FIG. 13B, LC66C6 reduces transpirational water loss over a period of 2 hours in detached leaves of the above species. Further, LC66C6 strongly induces expression of the ABA-responsive genes GmNAC4 and GmbZIP1 in soybeans (FIG. 13C), moderately induces expression of the ABA-responsive genes HVA1 and HvDRF1 in barley (FIG. 13D), and weakly induces expression of the ABA-responsive genes ZmRab17 and ZmLEA in maize (FIG. 13E).

This example demonstrates that LC66C6 inhibits germination and reduces transpirational water loss in a diverse group of agriculturally important species, indicating that LC66C6 is useful in reducing drought stress in multiple species.

### Example 7

This example shows the chemical structures of ABA and the agonists described herein, and the effect of the agonists in vitro and in vivo.

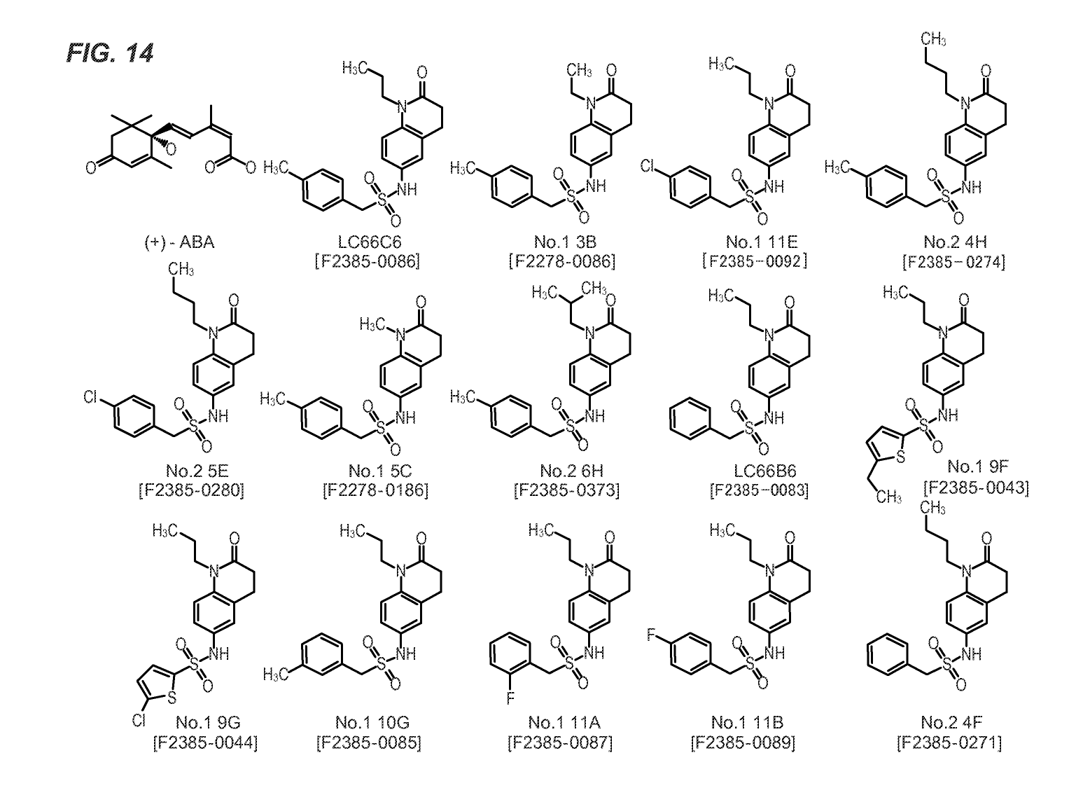

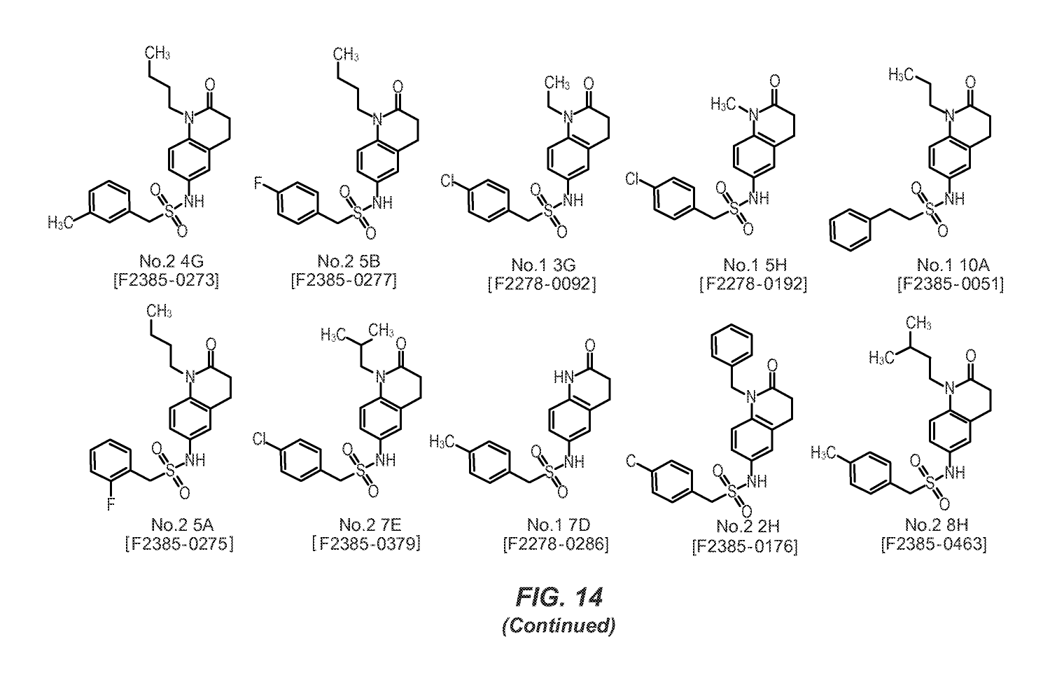

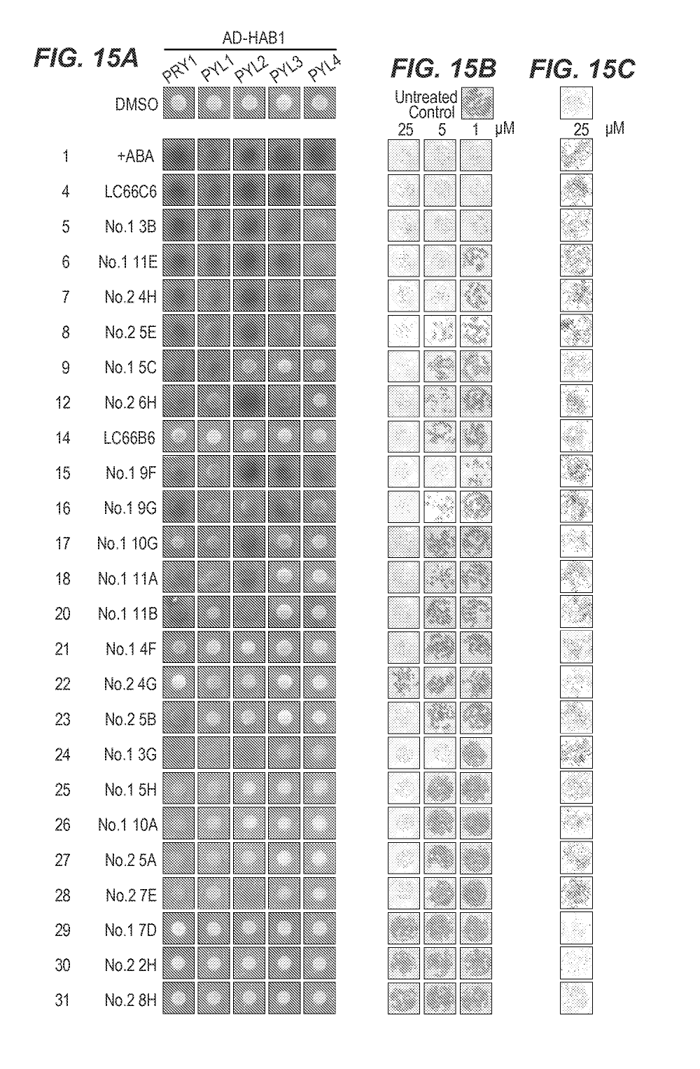

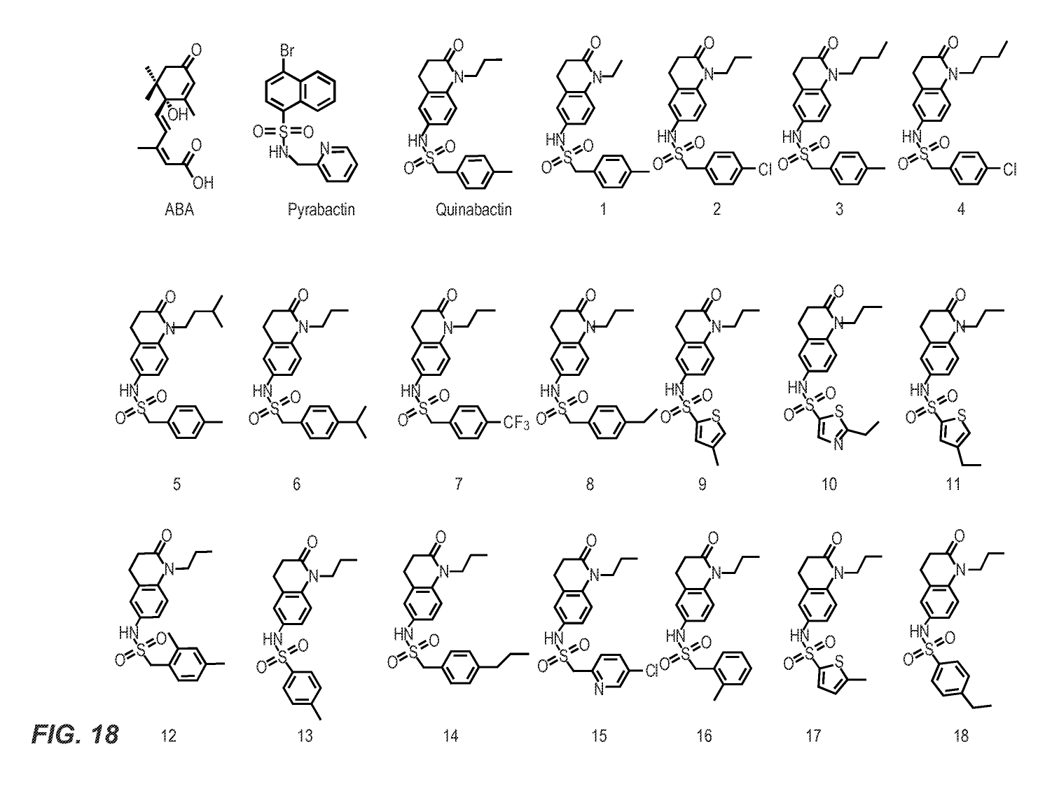

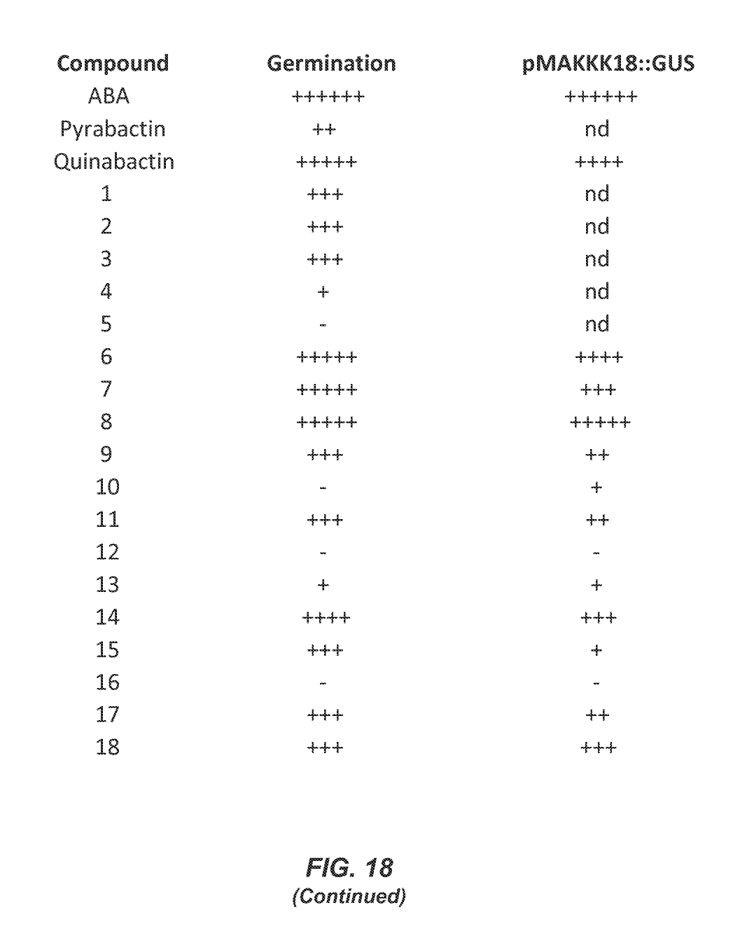

FIGS. 14 and 18 show the chemical structures of ABA and the agonists tested. FIG. 15A shows the results of yeast two-hybrid assays using PYR/PYL receptors PYR1, PYL1, PYL2, PYL3, and PYL4 to test the response to each of the agonists shown in FIG. 14. FIG. 15B shows the results of testing the agonists in FIG. 14 on germination of wild-type seeds, and demonstrates that LC66C6 is one of the most effective agonists, after (+)-ABA, at inhibiting germination of wild-type seeds. FIG. 15C shows the effects of compounds on an ABA-reporter line as measured using glucuronidase assays in a transgenic line expressing glucuronidase under the control of the ABA-inducible Arabidopsis gene MAPKKK18.

This example demonstrates that LC66C6 is one of the most effective agonists tested both in vitro and in vivo.

### Example 8

This example shows that LC66C6 can increase the size of ABA-deficient mutant plants.

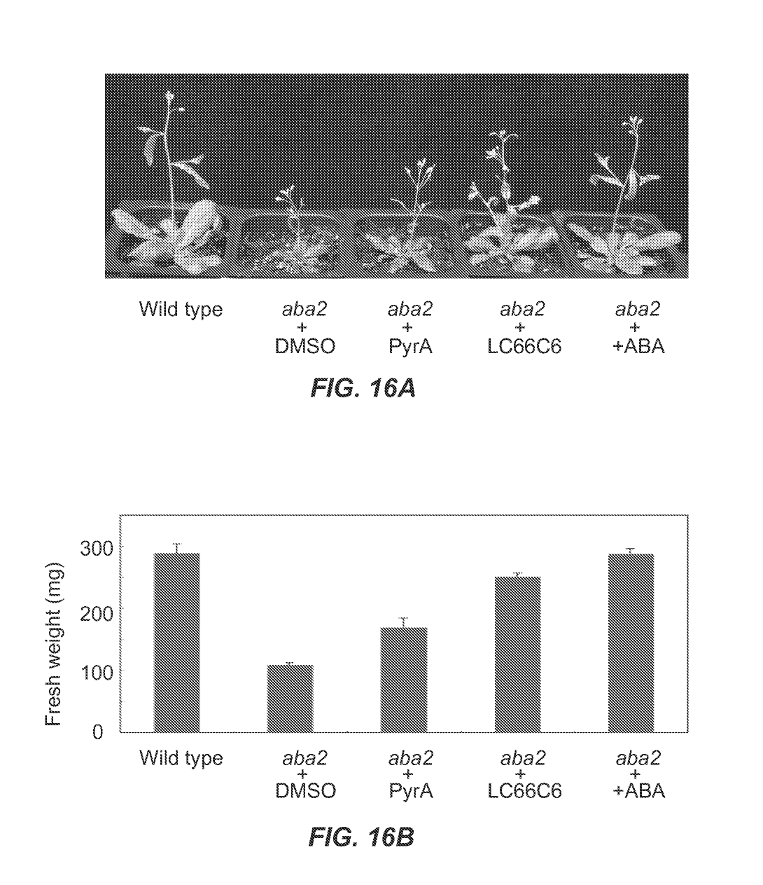

In this example, 14-day old wild-type and aba2 mutant plants were sprayed with a solution containing 25 μM of agonist two times a day for two weeks. Images and fresh weight were obtained from 4 week old plants. As shown in FIG. 16, application of LC66C6 to aba2 mutant plants significantly increased the size of the mutant plants compared to control plants treated with the carrier DMSO only.

This example demonstrates that LC66C6 can complement the growth phenotype observed in the aba2 mutation in a manner similar to that of (+)-ABA.

### Example 9

This example shows that LC66C6 can weakly inhibit protonema growth in moss, but has no effect on growth of the unicellular green algae Chlamydomonas.

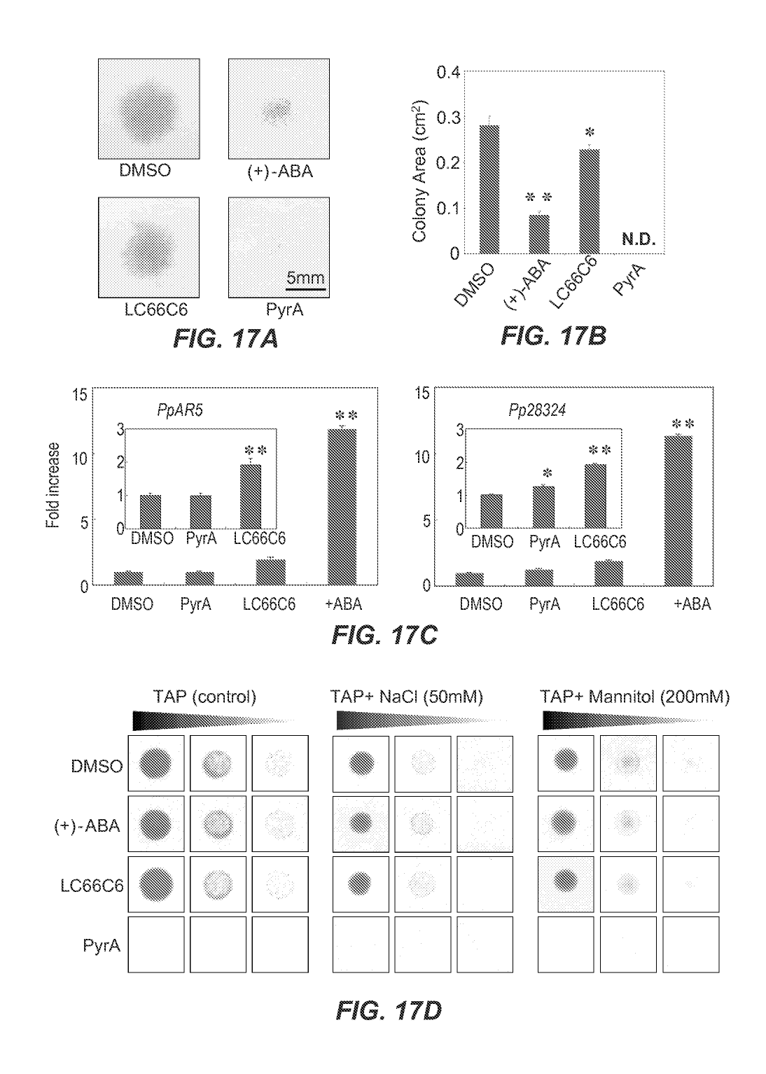

As shown in FIGS. 17A and 17B, LC66C6 showed a weak but significant inhibition on the growth of protonema of the moss Physcomitrella patens. Pyrabactin bleached the protonema, suggesting it might be toxic for this species.

FIG. 17C shows that LC66C6 can induce the expression of ABA-responsive genes in moss. However, these induction levels were weaker than those of ABA.

As shown in FIG. 17D, both (+)-ABA and LC66C6 had no effect on the growth of Chlamydomonas with and without salinity and osmotic stress. Again, pyrabactin bleached the Chlamydomonas, suggesting it is toxic to this species as well.

This example shows that LC66C6 can weakly inhibit protonemal growth and weakly induce ABA-responsive gene expression in the moss Physcomitrella patens, but does not effect the growth of the unicellular algae Chlamydomonas.

### Example 10

### Compound Synthesis

### 10.1 Preparation of Compound 1.001

### 1) 1-allyl-6-nitro-3,4-dihydroquinolin-2-one

6-nitro-3,4-dihydro-1H-quinolin-2-one (19.2 g) was dissolved in DMF (150 ml), cooled to 5° C. and K2CO3 (18.2 g) was added. 3-Bromopropene (15.7 g) was added drop wise and the reaction was stirred overnight at room temperature. The reaction mixture was poured into ice/water and the precipitated product was filtered and washed with water. The resulting wet crystals were stirred in ethanol (60 ml), and diethyl ether was added, the suspension was filtered again and the obtained filter cake was washed with diethyl ether and the dried under vacuum to give 21.7 g of product.

1H NMR (CDCl3, 400 MHz) β=8.10 (m, 2H), 7.08 (d, 1H), 5.85 (m, 1H), 5.25 (d, 1H), 5.12 (d, 1H), 4.60 (m, 2H), 3.05 (dd, 2H), 2.73 (dd, 2H).

### 2) 1-allyl-3-methyl-6-nitro-3,4-dihydroquinolin-2-one

1-allyl-6-nitro-3,4-dihydroquinolin-2-one (929 mg) was dissolved in dry THF (32 ml), degassed and cooled to −15° C. MeI (1.14 g) was added and then LiHMDS (4.4 ml of a 1M solution in THF) was added drop wise. The reaction was stirred for 20 min and poured onto NH4Cl (aq) and extracted twice with EtOAc. Organic layers were dried over Na2SO4, concentrated and purified by chromatography to give 886 mg of product.

1H NMR (CDCl3, 400 MHz) δ=8.10 (m, 2H), 7.03 (d, 1H), 5.85 (m, 1H), 5.22 (d, 1H), 5.12 (d, 1H), 4.60 (m, 2H), 3.05 (dd, 1H), 2.75 (m, 2H), 1.30 (d, 3H).

### 3) 1-allyl-6-amino-3-methyl-3,4-dihydroquinolin-2-one

1-allyl-3-methyl-6-nitro-3,4-dihydroquinolin-2-one (880 mg) was suspended in Ethanol (8.8 ml) and water (4.4 ml). NH4Cl (1.91 g) and Fe (reduced powder) (600 mg) was added and the reaction was heated to reflux. After 1.5 h NH4Cl (850 mg) and Fe (reduced powder) (300 mg) were added and refluxing continued for further 1.5 h. The reaction mixture was cooled, diluted with CH2Cl2 and filtered through celite. The filtrate was washed with CH2Cl2 and water. The solution was acidified with HCl (aq) and washed with twice CH2Cl2. The acidic aqueous phase were poured to an aqueous solution of K2CO3 and the resulting neutral water solution was extracted twice with CH2Cl2. Organic layers were concentrated to give 627 mg of product.

1H NMR (CDCl3, 400 MHz) δ=6.25 (d, 1H), 6.5 (m, 2H), 5.85 (m, 1H), 5.10 (m, 2H), 4.49 (m, 2H), 3.5 (bs, 2H), 2.9-2.5 (m, 3H), 1.22 (d, 3H).

### 4) Compound 1.001

1-allyl-6-amino-3-methyl-3,4-dihydroquinolin-2-one (130 mg) was dissolved in CH2Cl2 (3 ml) and cooled to 0° C. iPr2NEt (117 mg) and p-tolylmethanesulfonylchloride (129 mg) were added. While warming to room temperature, the reaction was stirred for 7 h, diluted with CH2Cl2 and washed with NaHCO3 (aq) and HCl (aq). The organic layer was concentrated and purified by chromatography to give 140 mg of product.

1H NMR (CDCl3, 400 MHz) δ=7.17 (m, 4H), 6.90 (m, 2H), 6.30 (s, 1H), 5.85 (m, 1H), 5.10 (m, 2H), 4.50 (m, 2H), 4.28 (s, 2H), 2.9-2.6 (m, 3H), 2.33 (s, 3H), 1.22 (d, 3H).

### 10.2 Preparation of Compound 15.001

Compound 15.001 was prepared in analogy to compound 1.001.

1H NMR (CDCl3, 400 MHz) δ=7.4-7.3 (m, 4H), 6.90 (m, 2H), 6.28 (s, 1H), 5.85 (m, 1H), 5.15 (m, 2H), 4.50 (m, 2H), 4.3 (s, 2H), 2.9-2.6 (m, 3H), 1.23 (d, 3H).

### 10.3 Preparation of Building Blocks

The following compounds can be used as building blocks in the preparation of compounds of the present invention.

### A. 1-allyl-6-amino-8-methyl-3,4-dihydroquinolin-2-one

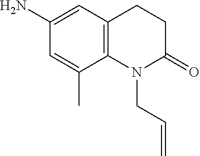 [SMILES: C=CCN1C(=O)CCc2cc(N)cc(C)c21]

### 1) N-(o-tolyl)-3-phenyl-prop-2-enamide

A solution of cinnamoyl chloride (181 g) in acetone (200 ml) was added drop wise to a cooled (−20° C.) solution of o-toluidine (107.7 g) in acetone (1 L) and ice (1 kg) and K2CO3 (153 g). After addition, the reaction mixture was stirred for 1 h, poured onto ice/water and the precipitate was filtered, washed with water and dried at 100° C. under vacuum to obtained 239 g or product

1H NMR (CDCl3, 400 MHz) δ=8.0-7.1 (m, 9H), 6.6 (bd, 1H), 4.8 (s, 2H), 2.3 (s, 3H)

### 2) 8-methyl-1H-quinolin-2-one

N-(o-tolyl)-3-phenyl-prop-2-enamide (9.5 g) and AlCl3 (17.8 g) were melted at 180° C. and then heated at 100° C. for 1 h. The resulting mixture was poured into water/ice (2 L) and the precipitating brownish solid was filtered and washed sequentially with water, HCl (aq), water and dried under vacuum at 100° C. to give 5.0 g or product.

1H NMR (CDCl3, 400 MHz) δ=9.2 (bs, 1H), 7.76 (d, 1H), 7.43 (d, 1H), 7.35 (d, 1H), 7.13 (dd, 1H), 6.65 (d, 1H), 2.45 (s, 3H)

### 3) 8-methyl-3,4-dihydro-1H-quinolin-2-one

8-methyl-1H-quinolin-2-one (108 g) was dissolved in AcOH (800 ml) and degassed. Under an argon atmosphere, 10% Pd/C (10.8 g) was added and the resulting mixture was placed under a hydrogen atmosphere (1 atm) and stirred at 90° C. for 10 h. The hydrogen atmosphere was exchanged with argon, and the reaction mixture was filtered through celite, and washed with EtOAc. Pd-waste was adequately disposed. The resulting solution was concentrated. Recrystallization of the crude material gave 51 g of product. The remaining mother liquid was diluted with EtOAc, washed with water and concentrated to give further 30 g of product.

1H NMR (CDCl3, 400 MHz) δ=7.55 (bs, 1H), 7.05 (m, 2H), 6.90 (dd, 1H), 2.95 (m, 2H), 2.63 (m, 2H), 2.21 (s, 3H)

### 4) 8-methyl-6-nitro-3,4-dihydro-1H-quinolin-2-one

8-methyl-3,4-dihydro-1H-quinolin-2-one (10 g) and sulfuric acid (186 ml) were mixed in a flask equipped with a mechanical stirrer. The clear solution was cooled to 0° C. and HNO3 (6.0 g) was added drop wise during 15 min, wise while vigorously stirring the reaction. Stirring was continued for 0.5 h, the reaction mixture was poured into ice/water and the suspension was filtered. Recrystallization of the crude material from EtOAc gave 9.1 g of product.

1H NMR (CDCl3, 400 MHz) δ=8.0 (m, 2H), 7.85 (bs, 1H), 3.05 (m, 2H), 2.70 (m, 2H), 2.31 (s, 3H).

### 5) 1-Allyl-8-methyl-6-nitro-3,4-dihydroquinolin-2-one

8-methyl-6-nitro-3,4-dihydro-1H-quinolin-2-one (3.0 g) was added to a suspension of NaH (1.45 g) in DMF (58 ml) at room temperature. After stirring this mixture for 20 min, allyl bromide (10.9 g) was added drop wise, the resulting mixture was stirred for 48 h, quenched with water and extracted with EtOAc. The organic phase was dried over Na2SO4, concentrated and purified by chromatography to give 2.59 of product.

1H NMR (CDCl3, 400 MHz) δ=7.95 (d, 1H), 7.9 (d, 1H), 5.7 (m, 1H), 5.1 (m, 2H), 4.58 (m, 2H), 2.94 (m, 2H, 2.63 (M, 2H), 2.41 (s, 3H).

### 6) 1-allyl-6-amino-8-methyl-3,4-dihydroquinolin-2-one

8-methyl-6-nitro-3,4-dihydro-1H-quinolin-2-one (2.6 g) was suspended in ethanol (26 ml) and water (13 ml). NH4Cl (8.44 g) and the reaction was heated to reflux. Fe (reduced powder) (2.94 g) was added in portions over a period of one hour. After 1.5 h, the reaction mixture was cooled, diluted with EtOAc, filtered through celite and the organic layers were concentrated and purified by chromatography to give 2.1 g of product.

1H NMR (CDCl3, 400 MHz) δ=6.39 (s, 2H), 5.72 (m, 1H), 5.1 (m, 2H), 4.48 (m, 2H), 3.5 (bs, 2H), 2.70 (m, 2H), 2.52 (m, 2H), 2.23 (s, 3H).

### B. 6-amino-8-methyl-1-prop-2-ynyl-3,4-dihydroquinolin-2-one

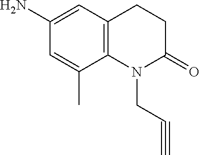 [SMILES: C#CCN1C(=O)CCc2cc(N)cc(C)c21]

### 1) 8-methyl-6-nitro-1-prop-2-ynyl-3,4-dihydroquinolin-2-one

8-methyl-6-nitro-3,4-dihydro-1H-quinolin-2-one (2.0 g) was added to a suspension of NaH (970 mg) in DMF (38 ml) at room temperature. After stirring this mixture for 20 min, propargyl bromide (6.48 ml of a 80% solution in toluene) was added drop wise, the resulting mixture was stirred for 16 h, quenched with water and extracted with EtOAc. The organic phase was dried over Na2SO4, concentrated and purified by chromatography to give 2.07 g of product.

1H NMR (CDCl3, 400 MHz) δ=8.03 (d, 1H), 7.93 (d, 1H), 4.72 (s, 2H), 2.95 (m, 2H), 2.65 (m, 2H), 2.56 (s, 3H), 2.20 (t, 1H).

### 2) 6-amino-8-methyl-1-prop-2-ynyl-3,4-dihydroquinolin-2-one

8-methyl-6-nitro-1-prop-2-ynyl-3,4-dihydroquinolin-2-one (2.07 g) was suspended in Ethanol (21 ml) and water (10.5 ml). NH4Cl (6.8 g) and the reaction was heated to reflux. Fe (reduced powder) (2.37 g) was added in portions over a period of one hour. After 1.5 h, the reaction mixture was cooled, diluted with EtOAc, filtered through celite and the organic layers were concentrated and purified by chromatography to give 1.32 g of product.

1H NMR (CDCl3, 400 MHz) δ=6.35 (m, 2H), 4.55 (s, 2H), 3.55 (s, 2H), 2.73 (m, 2H), 2.52 (m, 2H), 2.34 (s, 3H), 2.18 (t, 1H).

### 10.4 Coupling of building block A to prepare compound 1.079 (N-(8-methyl-2-oxo-1-prop-2-ynyl-3,4-dihydroquinolin-6-yl)-1-(p-tolyl)methanesulfonamide)

To a solution of 6-amino-8-methyl-1-prop-2-ynyl-3,4-dihydroquinolin-2-one (0.1 mmol) in ethyl acetate (0.5 ml) was added Hünig's base (0.15 mmol). The reaction mixture was cooled to 0° C. in an ice-ethanol bath. A solution of p-tolylmethanesulfonyl chloride (0.15 mmol) in ethyl acetate (0.75 ml) was added dropwise and the reaction mixture was stirred at ambient temperature for 4 hours. The reaction mixture was concentrated. The remaining mixture was diluted with N,N-dimethylacetamide (0.3 ml) and methanol (1.25 ml) and purified by HPLC to give N-(8-methyl-2-oxo-1-prop-2-ynyl-3,4-dihydroquinolin-6-yl)-1-(p-tolyl)methanesulfonamide, compound 1.079.

Compounds were identified by UPLC-MS: Retention time (RT)=1.33 min; M (calculated): 382.14; (M+H) (measured): 383.06

### UPLC-MS Conditions

Waters SQD2 Mass Spectrometer (Single quadrupole mass spectrometer)

Ionisation method: Electrospray

Polarity: positive ions

Capillary (kV) 3.50, Cone (V) 30.00, Extractor (V) 3.00, Source Temperature (° C.) 150, Desolvation Temperature (° C.) 400 Cone Gas Flow (L/Hr) 60, Desolvation Gas Flow (L/Hr) 700

Mass range: 140 to 800 Da

DAD Wavelength range (nm): 210 to 400

Method Waters ACQUITY UPLC with the following HPLC gradient conditions

(Solvent A: Water/Methanol 9:1, 0.1% formic acid and Solvent B: Acetonitrile, 0.1% formic acid)

| Time | A | B | Flow rate |
|---|---|---|---|
| (minutes) | (%) | (%) | (ml/min) |
| 0 | 100 | 0 | 0.75 |
| 2.5 | 0 | 100 | 0.75 |
| 2.8 | 0 | 100 | 0.75 |
| 3.0 | 100 | 0 | 0.75 |

### 10.5 Coupling of Building Blocks A or B to Prepare Other Compounds

Using 6-amino-8-methyl-1-prop-2-ynyl-3,4-dihydroquinolin-2-one and 1-allyl-6-amino-8-methyl-3,4-dihydroquinolin-2-one, the method described above was used to prepare compounds 6.007, 1.079, 4.007, 1.007, 15.007, 6.079 and 15.079 in parallel synthesis as shown in Table 3 below:

TABLE 3

| | | | | M | (M + H)⁺ |
|---|---|---|---|---|---|
| Compound | Structure | Formula | RT | (calculated) | (measured) |
| 6.007 | 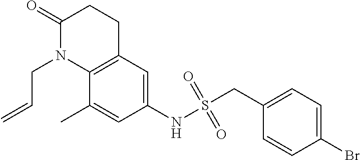 [SMILES: C=CCN1C(=O)CCc2cc(NS(=O)(=O)Cc3ccc(Br)cc3)cc(C)c21] | C20H21BrN2O3S | 1.45 | 448.1 | 448.94 |
| 1.079 | 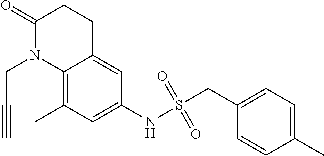 [SMILES: C#CCN1C(=O)CCc2cc(NS(=O)(=O)Cc3ccc(C)cc3)cc(C)c21] | C21H22N2O3S | 1.33 | 382.1 | 383.06 |
| 4.007 | 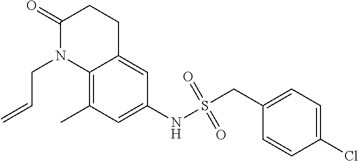 [SMILES: C=CCN1C(=O)CCc2cc(NS(=O)(=O)Cc3ccc(Cl)cc3)cc(C)c21] | C20H21ClN2O3S | 1.43 | 404.1 | 405.03 |
| 1.007 | 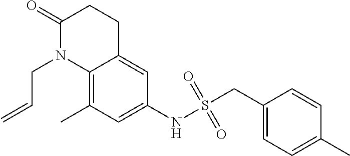 [SMILES: C=CCN1C(=O)CCc2cc(NS(=O)(=O)Cc3ccc(C)cc3)cc(C)c21] | C21H24N2O3S | 1.41 | 384.2 | 385.10 |
| 15.007 | 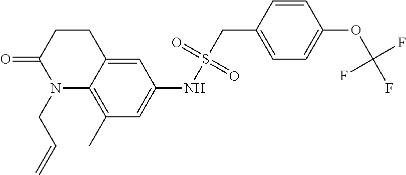 [SMILES: C=CCN1C(=O)CCc2cc(NS(=O)(=O)Cc3ccc(OC(F)(F)F)cc3)cc(C)c21] | C21H21F3N2O4S | 1.54 | 454.1 | 455.01 |
| 6.079 | 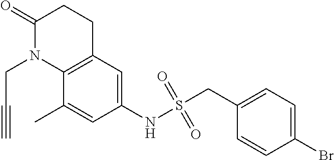 [SMILES: C#CCN1C(=O)CCc2cc(NS(=O)(=O)Cc3ccc(Br)cc3)cc(C)c21] | C20H19BrN2O3S | 1.38 | 446.0 | 446.93 |
| 15.079 | 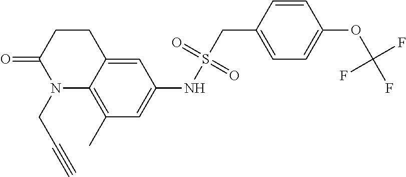 [SMILES: C#CCN1C(=O)CCc2cc(NS(=O)(=O)Cc3ccc(OC(F)(F)F)cc3)cc(C)c21] | C21H19F3N2O4S | 1.48 | 452.1 | 453.00 |

### Example 11

### PP2C Activity Assay

The protein HAB1, a type 2 protein phosphatase (PP2C), is inhibited by PYR/PYL proteins in dependence of abscisic acid or other antagonists. The potency of an antagonist correlates with the level of inhibition of the PP2C, and therefore the IC50 (PYR1-HAB1) can be used to compare the relative activity of different chemical analogues. Since inhibition of PP2C correlates to inhibition of seed-germination and increase in plant water-use efficiency, it serves as a powerful tool to quantify biological potential of a chemical acting as an analogue of abscisic acid.

HAB1 and PYL proteins were expressed and purified as described in Park et al. ((2009) Science 324(5930):1068-1071), with minor modifications. To obtain GST-HAB1, -ABI1 and -ABI2 fusion proteins, the HAB1 cDNA was cloned into pGex-2T whereas ABI1 and ABI2 cDNAs were cloned into the vector pGex-4T-1. Expression was conducted in BL21[DE3]pLysS host cells. Transformed cells were pre-cultured overnight, transferred to LB medium and cultured at 30° C. to culture A600 of ˜0.5.

The culture was then cooled on ice and MnCl2 added to 4 mM and IPTG added to 0.3 mM. After 16 hours incubation at 15° C., cells were harvested and recombinant proteins were purified on glutathione agarose as described in Park et al. To obtain 6×His-PYL receptor fusion proteins, receptor cDNAs for all 13 ABA receptors were cloned into the vector pET28 and expressed and purified as described in Mosquna et al. ((2011) PNAS 108(51):20838-20843); this yielded soluble and functional protein (assessed using receptor-mediated PP2C inhibition assays) for all receptors except PYL7, PYL11 and PYL12. These three receptors were therefore alternatively expressed as maltose binding (MBP) fusion proteins using the vector pMAL-c; expression of these constructs was carried out in BL21[DE3]pLysS host strain with the same induction conditions used for GST-HAB1. Recombinant MBP-PYL fusion proteins were purified from sonicated and cleared lysate using amylose resin (New England Biolab, Inc.) using the manufacturers purification instructions. This effort yielded an active MBP-PYL11 fusion protein, but failed for PYL7 and PYL12.

PP2C activity assays using recombinant receptors and PP2Cs were carried out as follows: Purified proteins were pre-incubated in 80 μl assay buffer containing 10 mM MnCl2, 3 μg bovine serum albumin and 0.1% 2-mercaptoethanol with ABA or ABA agonist (compounds of the present invention) for 30 minutes at 22° C. Reactions were started by adding 20 μL of a reaction solution containing 156 mM Tris-OAc, pH 7.9, 330 mM KOAc and 5 mM 4-methylumbelliferyl phosphate after which fluorescence measurements were immediately collected using an excitation filter 355 nm and an emission filter 460 nm on a Wallac plate reader. Reactions contained 50 nM PP2C and 100 nM PYR/PYL proteins, respectively. The results are expressed in Table 4.

TABLE 4
Inhibition of PP2C

| | IC50 |
|---|---|
| | (PYR1- |
| | HAB1) |
| Compound | nM |
| 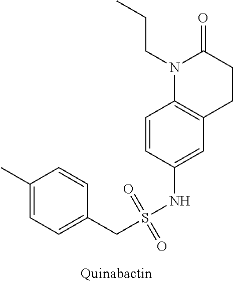 [SMILES: CCCN1C(=O)CCc2cc(NS(=O)(=O)Cc3ccc(C)cc3)ccc21] | 0.016 |
| 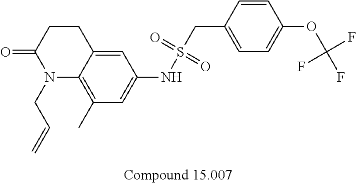 [SMILES: C=CCN1C(=O)CCc2cc(NS(=O)(=O)Cc3ccc(OC(F)(F)F)cc3)cc(C)c21] | 0.020 |
| 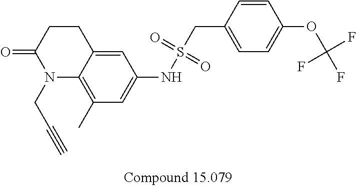 [SMILES: C#CCN1C(=O)CCc2cc(NS(=O)(=O)Cc3ccc(OC(F)(F)F)cc3)cc(C)c21] | 0.042 |
| 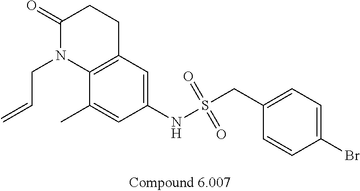 [SMILES: C=CCN1C(=O)CCc2cc(NS(=O)(=O)Cc3ccc(Br)cc3)cc(C)c21] | 0.034 |
| 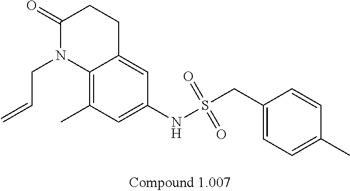 [SMILES: C=CCN1C(=O)CCc2cc(NS(=O)(=O)Cc3ccc(C)cc3)cc(C)c21] | 0.030 |
| 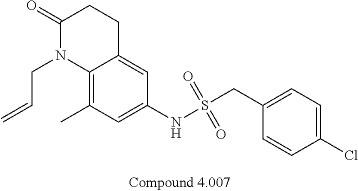 [SMILES: C=CCN1C(=O)CCc2cc(NS(=O)(=O)Cc3ccc(Cl)cc3)cc(C)c21] | 0.052 |
| 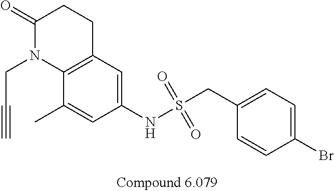 [SMILES: C#CCN1C(=O)CCc2cc(NS(=O)(=O)Cc3ccc(Br)cc3)cc(C)c21] | 0.069 |
| 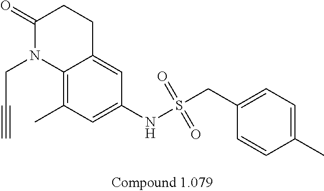 [SMILES: C#CCN1C(=O)CCc2cc(NS(=O)(=O)Cc3ccc(C)cc3)cc(C)c21] | 0.074 |

The results show that compounds of the present invention result in inhibition of PP2C at comparable levels to quinabactin.

### Example 12

### Arabidopsis Germination Inhibition Analysis

To analyse the effect of compounds on inhibition of germination, Arabidopsis seeds after-ripened for about 4 weeks were surface-sterilized with a solution containing 5% NaClO and 0.05% Tween-20 for 10 minutes, and rinsed with water four times. Sterilized seeds were suspended with 0.1% agar and sowed on 0.8% solidified agar medium containing ½ Murashige and Skoog (MS) salts (Sigma-Aldrich) in the presence of the relevant treatment, stored at 4° C. for 4 days, and then transferred to 22° C. under the dark conditions. Germination was assessed after 3 days.

TABLE 5
Percentage germination of Arabidopsis seeds

| Compound | 0.2 uM | 1 uM | 5 uM | 25 uM |
|---|---|---|---|---|
| 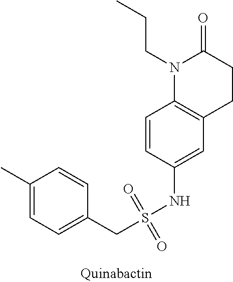 [SMILES: CCCN1C(=O)CCc2cc(NS(=O)(=O)Cc3ccc(C)cc3)ccc21] | 100 | 46 | 0 | 0 |
| 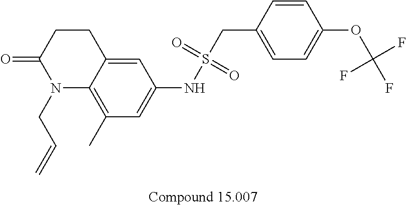 [SMILES: C=CCN1C(=O)CCc2cc(NS(=O)(=O)Cc3ccc(OC(F)(F)F)cc3)cc(C)c21] | 100 | 100 | 30 | 0 |
| 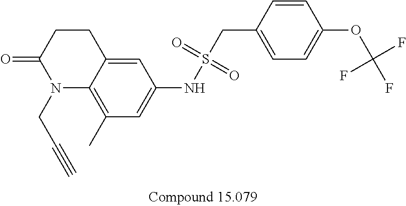 [SMILES: C#CCN1C(=O)CCc2cc(NS(=O)(=O)Cc3ccc(OC(F)(F)F)cc3)cc(C)c21] | 100 | 100 | 68 | 0 |
| 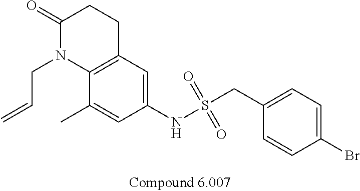 [SMILES: C=CCN1C(=O)CCc2cc(NS(=O)(=O)Cc3ccc(Br)cc3)cc(C)c21] | 100 | 100 | 96 | 0 |
| 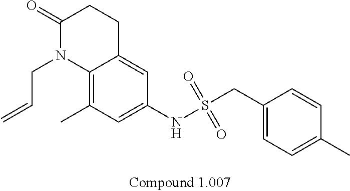 [SMILES: C=CCN1C(=O)CCc2cc(NS(=O)(=O)Cc3ccc(C)cc3)cc(C)c21] | 100 | 100 | 100 | 0 |
| 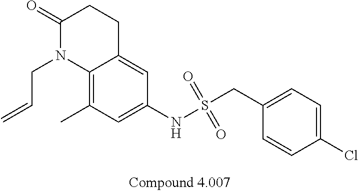 [SMILES: C=CCN1C(=O)CCc2cc(NS(=O)(=O)Cc3ccc(Cl)cc3)cc(C)c21] | 100 | 100 | 100 | 0 |
| 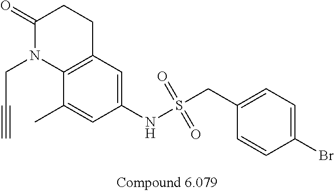 [SMILES: C#CCN1C(=O)CCc2cc(NS(=O)(=O)Cc3ccc(Br)cc3)cc(C)c21] | 100 | 100 | 100 | 0 |
| 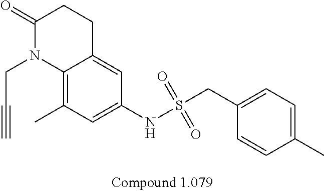 [SMILES: C#CCN1C(=O)CCc2cc(NS(=O)(=O)Cc3ccc(C)cc3)cc(C)c21] | 100 | 100 | 100 | 0 |
| 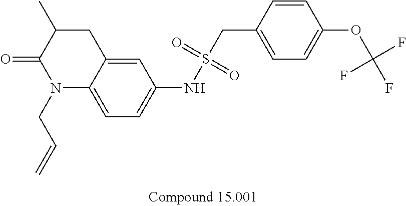 [SMILES: C=CCN1C(=O)C(C)Cc2cc(NS(=O)(=O)Cc3ccc(OC(F)(F)F)cc3)ccc21] | 100 | 100 | 100 | 14 |
| 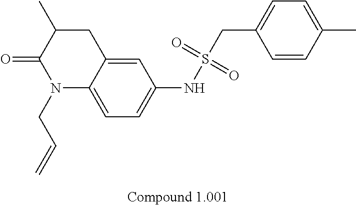 [SMILES: C=CCN1C(=O)C(C)Cc2cc(NS(=O)(=O)Cc3ccc(C)cc3)ccc21] | 99 | 100 | 100 | 71 |

The results show that compounds of the present invention inhibit germination of Arabidopsis seeds.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1. A compound of Formula I: 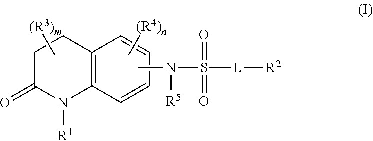
wherein
R¹ is selected from the group consisting of C₂₋₆ alkenyl, and C₂₋₆ alkynyl,
R² is selected from the group consisting of cycloalkyl, heterocycloalkyl, aryl and heteroaryl, each optionally substituted with from 1-4 R^{2a} groups,
each R^{2a} is independently selected from the group consisting of H, halogen, C₁₋₆ alkyl, C₁₋₆ alkoxy, C₁₋₆ haloalkyl, C₁₋₆ haloalkoxy, C₂₋₆ alkenyl, C₂₋₆ alkynyl, —OH, C₁₋₆ alkylhydroxy, —CN, —NO₂, —C(O)R^{2b}, —C(O)OR^{2b}, —OC(O)R^{2b}, —C(O)NR^{2b}R^{2c}, —NR^{2b}C(O)R^{2c}, —SO₂R^{2b}, —SO₂OR^{2b}, —SO₂NR^{2b}R^{2c}, and —NR^{2b}SO₂R^{2c},
each of R^{2b} and R^{2c} are independently selected from the group consisting of H and C₁₋₆ alkyl,
each of R³, R⁴ and R⁵ are independently selected from the group consisting of H and C₁₋₆ alkyl, wherein at least one R³ or R⁴ is methyl,
L is a linker selected from the group consisting of a bond and C₁₋₆ alkylene,
subscript m is an integer from 0 to 4,
subscript n is an integer from 0 to 3, and
m+n is greater than or equal to 1,
or a salt or isomer thereof.

2. The compound of claim 1, wherein the compound has the formula:

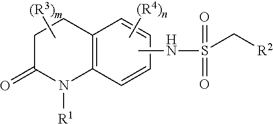

3. The compound of claim 2, wherein the compound has the formula:

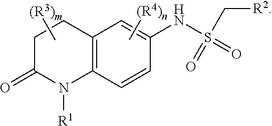

4. The compound of claim 2, wherein
R² is selected from the group consisting of aryl and heteroaryl, each optionally substituted with from 1-4 R^{2a} groups.

5. The compound of claim 4, wherein each R^{2a} is independently selected from the group consisting of H, halogen and C₁₋₆ alkyl.

6. The compound of claim 4, wherein R² is selected from the group consisting of phenyl, naphthyl, thiophene, furan, pyrrole, and pyridyl.

7. The compound of claim 4, wherein
R² is selected from the group consisting of phenyl and thiophene, each optionally substituted with 1 R^{2a} group;
each R^{2a} is independently selected from the group consisting of H, F, Cl, methyl, and ethyl; and
L is selected from the group consisting of a bond and methylene.

8. The compound of claim 7, wherein the compound has the formula:

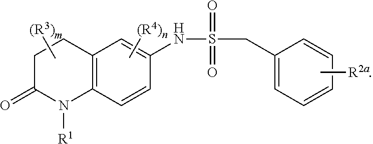

9. The compound of claim 7, wherein the compound has the formula:

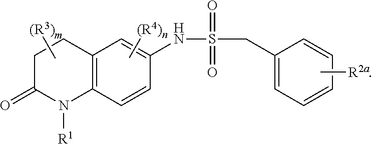

10. The compound of claim 1, wherein L is CH₂.

11. The compound of claim 1, wherein R⁵ is H.

12. The compound of claim 1, wherein R³ is CH₃.

13. The compound of claim 1, wherein R³ is CH₃ and R⁴ is H.

14. The compound of claim 1, wherein R³ is H and R⁴ is CH₃.

15. The compound of claim 1, wherein m is 2, and both R³ groups are CH3.

16. A compound as set forth in one of the Structures 1-59 having a combination of substituents as shown in any one row of Table 1.

17. An agricultural formulation comprising a compound of claim 1.

18. The formulation of claim 17, further comprising at least one of a fungicide, an herbicide, a pesticide, a nematicide, an insecticide, a plant activator, a synergist, an herbicide safener, a plant growth regulator, an insect repellant, an acaricide, a molluscicide, or a fertilizer.

19. The formulation of claim 17, further comprising a surfactant.

20. The formulation of claim 17, further comprising a carrier.

21. A method of increasing abiotic stress tolerance in a plant, the method comprising contacting a plant with a sufficient amount of the compound of claim 1 to increase abiotic stress tolerance in the plant compared to not contacting the plant with the formulation.

22. The method of claim 21 wherein the plant is a monocot.

23. The method of claim 21, wherein the plant is a dicot.

24. The method of claim 21, wherein the abiotic stress tolerance comprises drought tolerance.

25. The method of claim 21, wherein the contacting step comprises delivering the formulation to the plant by aircraft or irrigation.

26. A method of inhibiting seed germination in a plant, the method comprising contacting a seed with a sufficient amount of the compound of claim 1 to inhibit germination.

27. A plant in contact with the compound of claim 1.

28. The plant of claim 21, wherein the plant is a seed.

29. A method of activating a PYR/PYL protein, the method comprising contacting the PYR/PYL protein with the compound of claim 1 to 20.

30. The method of claim 29, wherein the PYR/PYL protein is expressed by a cell.

31. The method of claim 30, wherein the cell is a plant cell.

32. The method of claim 30, wherein the PYR/PYL protein is an endogenous protein.

33. The method of claim 30, wherein the PYR/PYL protein is a heterologous protein.

34. The method of claim 30, wherein the cell further expresses a type 2 protein phosphatase (PP2C).

35. The method of claim 34, wherein the type 2 protein phosphatase is HAB1 (Homology to ABI1), ABI1 (Abscisic acid insensitive 1), or ABI2 (Abscisic acid insensitive 2).
